(19) United States
(12) Patent Application Publication
(10) Pub. No.: US 2018/0185375 A1
(43) Pub. Date: Jul. 5, 2018

(54) COMPOSITIONS AND METHODS FOR THE TREATMENT OF SUBSTANCE USE DISORDERS, ADDICTION, AND PSYCHIATRIC DISORDERS

(71) Applicant: EMBERA NEUROTHERAPEUTICS, INC., Sudbury, MA (US)

(72) Inventors: Michael Detke, Carmel, IN (US); Martin Phillips, Bryn Mawr, PA (US); Robert Linke, Sudbury, MA (US)

(73) Assignee: Embera Neurotherapeutics, Inc., Sudbury, MA (US)

(21) Appl. No.: 15/739,005

(22) PCT Filed: Jun. 22, 2016
(86) PCT No.: PCT/US2016/038722

§ 371 Date: Dec. 21, 2017

Related U.S. Application Data
(60) Provisional application No. 62/182,789, filed on Jun. 22, 2015.

(51) Int. Cl. A61K 31/53; A61K 31/519; A61K 31/4196; A61K 31/4188; A61K 31/343; A61K 31/138; A61K 31/496; A61K 31/506; A61K 31/4168; A61P 25/30

(52) U.S. Cl. CPC A61K 31/53; A61K 31/519; A61K 31/4196; A61K 31/4188; A61P 25/30; A61K 31/138; A61K 31/496; A61K 31/506; A61K 31/4168; A61K 31/343

(57) ABSTRACT

The present invention features a composition comprising a first agent and a second agent for treating disorder associated with aberrant activity in the HPA axis like an addiction to a substance (e.g., cocaine, amphetamines, methamphetamine, methylphenidate, heroin, codeine, hydrocodone, nicotine, alcohol, prescription medication (e.g., Percodan®, Percoset®), marijuana, tobacco, methadone, food), addiction to an activity (e.g., gambling, sex, eating), substance use disorders, mood disorders, anxiety disorders, bipolar disorder, sleep disorders, insomnia, posttraumatic stress syndrome, borderline personality disorder, disruptive behavior disorders, ADHD, major depressive disorder, burnout, chronic fatigue syndrome, fibromyalgia, irritable bowel syndrome, eating disorders (e.g., Prader Willi Syndrome), obesity, depression, menopause, premenstrual syndrome (PMS), obsessive compulsive disorder (OCD), social anxiety, generalized anxiety disorder, dysthymia, or schizophrenia.

### TECHNICAL FIELD

This invention relates to pharmaceutical compositions useful for treating disorders associated with aberrant activity in the HPA axis, such as addiction to a substance, an addiction to an activity, mood disorders, anxiety disorders, bipolar disorder, insomnia, posttraumatic stress syndrome, borderline personality disorder, ADHD, major depressive disorder, burnout, chronic fatigue syndrome, fibromyalgia, irritable bowel syndrome, obesity, depression, or schizophrenia.

### SUMMARY OF THE INVENTION

The current invention relates to pharmaceutical compositions comprising a first agent and a second agent that are independently selected, and methods of using said compositions. The first aspect of this invention provides a pharmaceutical composition comprising a first agent and a second agent, wherein the first agent is mitotane, aminoglutethimide, etomidate, a compound of Formula I or a pharmaceutically acceptable salt, solvate, hydrate, polymorph, precursor, metabolite, prodrug or a derivative thereof;

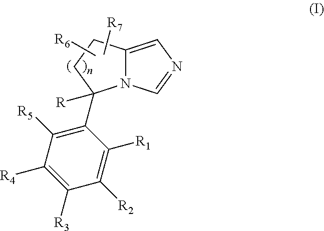

wherein

R is selected from the group consisting of: hydrogen, C₁-C₇ alkyl, and C₂-C₇ alkenyl,

R₁ is selected from F, Cl, Br and I,

R₂, R₃, R₄, and R₅ are selected independently from the group consisting of hydrogen, C₂-C₇ alkenyl, C₁-C₇ alkyl, C₃-C₈ cycloalkyl, F, Cl, Br, I, cyano, nitro, H₂N—, C₁-C₇ haloalkyl, and C₁-C₇ alkoxy,

R₆, and R₇ are hydrogen,

or a pharmaceutically acceptable salt thereof, or a pharmaceutically acceptable solvate, hydrate, polymorph, precursor, metabolite, or prodrug thereof;

wherein the second agent is selected from the group consisting of sedative, hypnotic, anxiolytic and anticonvulsant.

In some embodiments, R₂, R₃, R₄, and R₅ are independently selected from hydrogen, F, Cl, Br, I, cyano or C₁-C₄ alkyl . In yet other embodiments, the first agent is (R)-4-(6,7-dihydro-5H-pyrrolo[1,2-c]imidazol-5-yl)-3-fluorobenzonitrile. In some embodiments, the first agent is the compound of Formula II

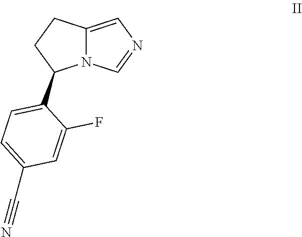 [SMILES: N#Cc1ccc([C@H]2CCc3cncn32)c(F)c1]

or an analog, enantiomer, a pharmaceutically acceptable salt, solvate, hydrate, polymorph, precursor, metabolite, or prodrug thereof.

In some embodiments, the first agent is mitotane or a pharmaceutically acceptable salt, solvate, hydrate, polymorph, precursor, metabolite, prodrug or a derivative thereof. In some embodiments, the first agent is aminoglutethimide or a pharmaceutically acceptable salt, solvate, hydrate, polymorph, precursor, metabolite, prodrug or a derivative thereof. In yet other embodiments, the first agent is etomidate or a pharmaceutically acceptable salt, solvate, hydrate, polymorph, precursor, metabolite, prodrug or a derivative thereof.

In some embodiments, the first agent is ketoconazole, 2S,4R enantiomer of ketoconazole, or a pharmaceutically acceptable salt, solvate, hydrate, polymorph, precursor, metabolite, prodrug or a derivative thereof.

In some embodiments, the second agent is a benzodiazepine. In some embodiments, the benzodiazepine may be selected from the group consisting of adinazolam, alprazolam, clonazepam, chlordiazepoxide, climazolam, clorazepate, diazepam, estazolam, flunitrazepam, flurazepam, halazepam, loprazolam, lorazepam, lormetazepam, midazolam, nimetazepam, nitrazepam, oxazepam, prazepam, temazepam, triazolam or a pharmaceutically acceptable salt thereof. The methods described herein may include or exclude any of the listed agents. In some embodiments, the second agent is oxazepam, chlordiazepoxide or a pharmaceutically acceptable salt thereof.

In some embodiments, the first agent is (R)-4-(6,7-dihydro-5H-pyrrolo[1,2-c]imidazol-5-yl)-3-fluorobenzonitrile, the compound of Formula 2

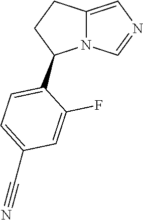 [SMILES: N#Cc1ccc([C@H]2CCc3cncn32)c(F)c1]

or an analog, pharmaceutically acceptable salt, solvate, hydrate, polymorph, precursor, metabolite, or prodrug thereof and the second agent is oxazepam, chlordiazepoxide or a pharmaceutically acceptable salt thereof.

In another aspect, the current invention relates to pharmaceutical compositions comprising a first agent and a second agent that are independently selected, and methods of using said compositions. In another aspect, this invention provides a pharmaceutical composition comprising a first agent and a second agent. In another embodiment, the first agent is an agent that inhibits or is shown to inhibit the HPA axis. In another embodiment, the second agent is an agent that possesses or is shown to possess anti-anxiolitic properties.

In another aspect, the first agent is a CRH/CRF-1 antagonist; an ACTH antagonist; or a cortisol inhibitor. As defined herein, the term cortisol inhibitor encompasses agents that inhibit the production of cortisol as well as agents that inhibit the activity of cortisol. Exemplary CRH/CRF-1 antagonists are selected from, but not limited to, antalarmin; pexacerfont; verucerfont; LWH-234; CP-154,536; NBI-27914; and R-121,919. Exemplary ACTH antagonists are selected from, but not limited to, bromocriptine; cabergoline; somastatin analogs (e.g., octreotide, pasireotide); retinoic acid; and cyproheptadine. Exemplary cortisol inhibitors are selected from, but not limited to, metyrapone; ketoconazole; mitotane; aminoglutethimide; etomidate; mifepristone; cytadren; and fluconazole.

In another aspect, the second agent is an antidepressant; benzodiazepine; beta-blocker; antipsychotic; alpha-adrenergic agonist; 5-HT1A agonist; azapirone; mebicarum; fabomitizole; selank; bromantane; emoxypine; hydroxyzine; pregbalin; methyl isovalerate; cannabidiol; tetrahydrocannabinol; propofol; BNC210; CL-218,872; L-838,417; SL-651,498; 532212; or PH94B.

In another aspect, the antidepressant is selected from serotonin selective reuptake inhibitors (SSRIs); serotonin norepinephrine reuptake inhibitors (SNRIs); serotonin modulators and stimulators (SMSs); serotonin antagonists and reuptake inhibitors (SARIs); norepinephrine reuptake inhibitors (NRIs); tricyclic antidepressants (TCAs); tetracyclic antidepressants (TeCAs); monoamine oxidase inhibitors (MAOIs); norepinephrine dopamine reuptake inhibitors; agomelatine; bifemelane; tandospirone; and teniloxazine. In another aspect, the antidepressant is selected from serotonin selective reuptake inhibitors (SSRIs); serotonin norepinephrine reuptake inhibitors (SNRIs); serotonin modulators and stimulators (SMSs); agomelatine; bifemelane; tandospirone; and teniloxazine.

Exemplary SSRIs are selected from, but not limited to, citalopram; escitalopram; paroxetine; fluoxetine; fluvoxamine; and sertraline. Exemplary SNRIs are selected from, but not limited to, desvenlafaxine; duloxetine; levomilnacipran; milnacipran; tofenacin; and venlafaxine. Exemplary SMSs are selected from, but not limited to, vilazodone and vortioxetine. Exemplary SARIs are selected from, but not limited to, etoperidone; nefazodone; and trazodone. Exemplary NRIs are selected from, but not limited to, reboxetine; viloxazine; and atromoxetine. Exemplary TCAs are selected from, but not limited to, amitriptyline; amitriptylinoxide; clomipramine; desipramine; dibenzepin; dimetacrine; dosulepin; doxepin; imipramine; lofepramine; melitracen; nitroxazepine; nortriptyline; noxiptiline; pipofezine; proptriptyline; trimipramine; opipramol; and tianeptine. Exemplary TeCAs are selected from, but not limited to, amoxapine; maprotiline; mianserin; mirtazapine; and setiptiline. Exemplary MAOIs are selected from, but not limited to, isocarboxazid; phenelzine; tranylcypromine; selegiline; metralindole; moclobemide; pirlindole; and toloxatone.

Exemplary benzodiazepines are selected from, but not limited to, oxazepam; chlordiazepoxide; mirtazapine; atomoxetine; gabapentin; (Neurontin™); muscimol; progabide; riluzole; baclofen; vigabatrin; valproic acid (Depakote™); tiagabine (Gabitril™); lamotrigine (Lamictal™); phenytoin (Dilantin™); carbamazepine (Tegretol™); topiramate (Topamax™); lorazepam; (Ativan®), prazepam (Centrax®); flurazepam (Dalmane®); clonazepam (Klonopin®); chlordiazepoxide (Librium®); halazepam (Paxipam®); temezepam (Restoril®); clorazapate; (Tranxene®), diazepam (Valium®), and alprazolam (Xanax®).

Exemplary beta-blockers are selected from, but not limited to, propanolol; bucindolol; carteolol; carvedilol; labetol; nadolol; oxprenolol; penbutolol; pindolol; sotalol; timolol; acebutolol; atenolol; betaxolol; bisoprolol; celiprolol; esmolol; metoprolol; nebivolol; butaxamine; ICI-118,551; and SR-59230A.

Exemplary antipsychotics are selected from, but not limited to, benperidol; bromperidol; droperidol; haloperidol; timiperone; diphenylbutylpiperidine; fluspirilene; penfluridol; pimozide; phenothiazines; chlorpromazine; cyamemazine; dixyrazine; fluphenazine; levomepromazine; perazine; pericyazine; perphenazine; pipotiazine; prochlorperazine; promethazine; prothipendyl; thioproperazine; trifluoperazine; chlorprothixene; clopenthixol; flupentixol; thiothixene; zuclopenthixol; clotiapine; loxapine; prothipendyl; carpipramine; clocapramine; molindone; mosapramine; sulpiride; sultopride; veralipride; amisulpride; amoxapine; arpiprazole; asenapine; cariprazine; clozapine; blonaserin; iloperidone; lurasidone; melperone; nemonapride; olanzapine; paliperidone; perospirone; quetiapine; remoxapride; risperidone; sertindole; trimipramine; ziprasidone; zotepine; brexpiprazole; ITI-007; pimavanserin; and RP5063.

Exemplary alpha-adrenergic agonists are selected from, but not limited to, clonidine and guanfacine.

Exemplary azapirones are selected from, but not limited to, buspirone and tandospirone.

In another aspect, the first agent is a CRH/CRF-1 antagonist, an ACTH antagonist, or a cortisol inhibitor; and the second agent is an antidepressant; benzodiazepine; beta-blocker; antipsychotic; alpha-adrenergic agonist; 5-HT 1 A agonist; azapirone; mebicarum; fabomitizole; selank; bromantane; emoxypine; hydroxyzine; pregbalin; methyl isovalerate; cannabidiol; tetrahydrocannabinol; propofol; BNC210; CL-218,872; L-838,417; SL-651,498; S32212; or PH94B. In another aspect, the first agent is a CRH/CRF-1 antagonist, an ACTH antagonist, or a cortisol inhibitor; and the second agent is an antidepressant; beta-blocker; antipsychotic; alpha-adrenergic agonist; or azapirone. In another aspect, the first agent is a CRH/CRF-1 antagonist and the second agent is an antidepressant. In another aspect, the first agent is a CRH/CRF-1 antagonist and the second agent is a benzodiazepine. In another aspect, the first agent is a CRH/CRF-1 antagonist and the second agent is a beta-blocker. In another aspect, the first agent is a CRH/CRF-1 antagonist and the second agent is an antipsychotic. In another aspect, the first agent is a CRH/CRF-1 antagonist and the second agent is an alpha-adrenergic agonist. In another aspect, the first agent is a CRH/CRF-1 antagonist and the second agent is an azapirone. In another aspect, the first agent is an ACTH antagonist and the second agent is an antidepressant. In another aspect, the first agent is an ACTH antagonist and the second agent is a benzodiazepine. In another aspect, the first agent is an ACTH antagonist and the second agent is a beta-blocker. In another aspect, the first agent is an ACTH antagonist and the second agent is an antipsychotic. In another aspect, the first agent is an ACTH antagonist and the second agent is an alpha-adrenergic agonist. In another aspect, the first agent is an ACTH antagonist and the second agent is an azapirone. In another aspect, the first agent is a cortisol inhibitor; and the second agent is an antidepressant. In another aspect, the first agent is a cortisol inhibitor; and the second agent is a benzodiazepine. In another aspect, the first agent is a cortisol inhibitor; and the second agent is a beta-blocker. In another aspect, the first agent is a cortisol inhibitor; and the second agent is an antipsychotic. In another aspect, the first agent is a cortisol inhibitor; and the second agent is an alpha-adrenergic agonist. In another aspect, the first agent is a cortisol inhibitor; and the second agent is an azapirone.

In another aspect, the first agent is a CRH/CRF-1 antagonist and the second agent is an antidepressant. In another aspect, the first agent is selected from antalarmin; pexacerfont; verucerfont; LWH-234; CP-154,536; NBI-27914; and R-121,919; and the second agent is an antidepressant. In another aspect, the first agent is selected from verucerfont; pexacerfont; LWH-234; and R-121,919; and the second agent is an antidepressant. In another aspect, the first agent is a CRH/CRF-1 antagonist and the second agent is an SSRI; SNRI; SMS; SARI; NRI; TCA; TeCA; MAOI; norepinephrine dopamine reuptake inhibitor; agomelatine; bifemelane; tandospirone; and teniloxazine. In another aspect, the first agent is selected from antalarmin; pexacerfont; verucerfont; LWH-234; CP-154,536; NBI-27914; and R-121,919; and the second agent is an SSRI; SNRI; SMS; bupropion; tandospirone; and teniloxazine. In another aspect, the first agent is selected from antalarmin; pexacerfont; verucerfont; LWH-234; CP-154,536; NBI-27914; and R-121,919; and the second agent is an SSRI; SNRI; SMS; SARI; NRI; TCA; TeCA; MAOI; norepinephrine dopamine reuptake inhibitor; agomelatine; bifemelane; tandospirone; and teniloxazine. In another aspect, the first agent is selected from antalarmin; pexacerfont; verucerfont; LWH-234; CP-154,536; NBI-27914; and R-121,919; and the second agent is an SSRI; SNRI; SMS; bupropion; tandospirone; and teniloxazine. In another aspect, the first agent is selected from verucerfont; pexacerfont; LWH-234; and R-121,919; and the second agent is an SSRI; SNRI; SMS; SARI; NRI; TCA; TeCA; MAOI; norepinephrine dopamine reuptake inhibitor; agomelatine; bifemelane; tandospirone; and teniloxazine. In another aspect, the first agent is selected from verucerfont; pexacerfont; LWH-234; and R-121,919; and the second agent is an SSRI; SNRI; SMS; bupropion; tandospirone; and teniloxazine. In another aspect, the first agent is selected from antalarmin; pexacerfont; verucerfont; LWH-234; CP-154,536; NBI-27914; and R-121,919; and the second agent is citalopram; escitalopram; paroxetine; fluoxetine; fluvoxamine; sertraline; desvenlafaxine; duloxetine; levomilnacipran; milnacipran; tofenacin; venlafaxine; vilazodone; vortioxetine; etoperidone; nefazodone; trazodone; reboxetine; viloxazine; atromoxetine; amitriptyline; amitriptylinoxide; clomipramine; desipramine; dibenzepin; dimetacrine; dosulepin; doxepin; imipramine; lofepramine; melitracen; nitroxazepine; nortriptyline; noxiptiline; pipofezine; proptriptyline; trimipramine; opipramol; tianeptine; amoxapine; maprotiline; mianserin; mirtazapine; setiptiline; isocarboxazid; phenelzine; tranylcypromine; selegiline; metralindole; moclobemide; pirlindole; toloxatone; bupropion; agomelatine; bifemelane; tandospirone; and teniloxazine. In another aspect, the first agent is selected from verucerfont; pexacerfont; LWH-234; and R-121,919; and the second agent is citalopram; escitalopram; paroxetine; fluoxetine; fluvoxamine; sertraline; desvenlafaxine; duloxetine; levomilnacipran; milnacipran; tofenacin; venlafaxine; vilazodone; vortioxetine; etoperidone; nefazodone; trazodone; reboxetine; viloxazine; atromoxetine; amitriptyline; amitriptylinoxide; clomipramine; desipramine; dibenzepin; dimetacrine; dosulepin; doxepin; imipramine; lofepramine; melitracen; nitroxazepine; nortriptyline; noxiptiline; pipofezine; proptriptyline; trimipramine; opipramol; tianeptine; amoxapine; maprotiline; mianserin; mirtazapine; setiptiline; isocarboxazid; phenelzine; tranylcypromine; selegiline; metralindole; moclobemide; pirlindole; toloxatone; bupropion; agomelatine; bifemelane; tandospirone; and teniloxazine. In another aspect, the first agent is selected from antalarmin; pexacerfont; verucerfont; LWH-234; CP-154,536; NBI-27914; and R-121,919; and the second agent is citalopram; escitalopram; paroxetine; fluoxetine; fluvoxamine; sertraline; desvenlafaxine; duloxetine; levomilnacipran; milnacipran; tofenacin; venlafaxine; vilazodone; vortioxetine; bupropion; agomelatine; bifemelane; tandospirone; and teniloxazine. In another aspect, the first agent is selected from verucerfont; pexacerfont; LWH-234; and R-121,919; and the second agent is citalopram; escitalopram; paroxetine; fluoxetine; fluvoxamine; sertraline; desvenlafaxine; duloxetine; levomilnacipran; milnacipran; tofenacin; venlafaxine; vilazodone; vortioxetine; bupropion; agomelatine; bifemelane; tandospirone; and teniloxazine.

In another aspect, the first agent is verucerfont; and the second agent is citalopram. In another aspect, the first agent is verucerfont; and the second agent is escitalopram. In another aspect, the first agent is verucerfont; and the second agent is paroxetine. In another aspect, the first agent is verucerfont; and the second agent is fluoxetine. In another aspect, the first agent is verucerfont; and the second agent is fluvoxamine. In another aspect, the first agent is verucerfont; and the second agent is sertraline. In another aspect, the first agent is verucerfont; and the second agent is desvenlafaxine. In another aspect, the first agent is verucerfont; and the second agent is duloxetine. In another aspect, the first agent is verucerfont; and the second agent is levomilnacipran. In another aspect, the first agent is verucerfont; and the second agent is milnacipran. In another aspect, the first agent is verucerfont; and the second agent is tofenacin. In another aspect, the first agent is verucerfont; and the second agent is tofenacin. In another aspect, the first agent is verucerfont; and the second agent is venlafaxine. In another aspect, the first agent is verucerfont; and the second agent is vilazodone. In another aspect, the first agent is verucerfont; and the second agent is vortioxetine. In another aspect, the first agent is verucerfont; and the second agent is bupropion. In another aspect, the first agent is verucerfont; and the second agent is agomelatine. In another aspect, the first agent is verucerfont; and the second agent is bifemelane. In another aspect, the first agent is verucerfont; and the second agent is tandospirone. In another aspect, the first agent is verucerfont; and the second agent is teniloxazine.

In another aspect, the first agent is pexacerfont; and the second agent is citalopram. In another aspect, the first agent is pexacerfont; and the second agent is escitalopram. In another aspect, the first agent is pexacerfont; and the second agent is paroxetine. In another aspect, the first agent is pexacerfont; and the second agent is fluoxetine. In another aspect, the first agent is pexacerfont; and the second agent is fluvoxamine. In another aspect, the first agent is pexacerfont; and the second agent is sertraline. In another aspect, the first agent is pexacerfont; and the second agent is desvenlafaxine. In another aspect, the first agent is pexacerfont; and the second agent is duloxetine. In another aspect, the first agent is pexacerfont; and the second agent is levomilnacipran. In another aspect, the first agent is pexacerfont; and the second agent is milnacipran. In another aspect, the first agent is pexacerfont; and the second agent is tofenacin. In another aspect, the first agent is pexacerfont; and the second agent is tofenacin. In another aspect, the first agent is pexacerfont; and the second agent is venlafaxine. In another aspect, the first agent is pexacerfont; and the second agent is vilazodone. In another aspect, the first agent is pexacerfont; and the second agent is vortioxetine. In another aspect, the first agent is pexacerfont; and the second agent is bupropion. In another aspect, the first agent is pexacerfont; and the second agent is agomelatine. In another aspect, the first agent is pexacerfont; and the second agent is bifemelane. In another aspect, the first agent is pexacerfont; and the second agent is tandospirone. In another aspect, the first agent is pexacerfont; and the second agent is teniloxazine.

In another aspect, the first agent is LWH-234; and the second agent is citalopram. In another aspect, the first agent is LWH-234; and the second agent is escitalopram. In another aspect, the first agent is LWH-234; and the second agent is paroxetine. In another aspect, the first agent is LWH-234; and the second agent is fluoxetine. In another aspect, the first agent is LWH-234; and the second agent is fluvoxamine. In another aspect, the first agent is LWH-234; and the second agent is sertraline. In another aspect, the first agent is LWH-234; and the second agent is desvenlafaxine. In another aspect, the first agent is LWH-234; and the second agent is duloxetine. In another aspect, the first agent is LWH-234; and the second agent is levomilnacipran. In another aspect, the first agent is LWH-234; and the second agent is milnacipran. In another aspect, the first agent is LWH-234; and the second agent is tofenacin. In another aspect, the first agent is LWH-234; and the second agent is tofenacin. In another aspect, the first agent is LWH-234; and the second agent is venlafaxine. In another aspect, the first agent is LWH-234; and the second agent is vilazodone. In another aspect, the first agent is LWH-234; and the second agent is vortioxetine. In another aspect, the first agent is LWH-234; and the second agent is bupropion. In another aspect, the first agent is LWH-234; and the second agent is agomelatine. In another aspect, the first agent is LWH-234; and the second agent is bifemelane. In another aspect, the first agent is LWH-234; and the second agent is tandospirone. In another aspect, the first agent is LWH-234; and the second agent is teniloxazine.

In another aspect, the first agent is R-121,919; and the second agent is citalopram. In another aspect, the first agent is R-121,919; and the second agent is escitalopram. In another aspect, the first agent is R-121,919; and the second agent is paroxetine. In another aspect, the first agent is R-121,919; and the second agent is fluoxetine. In another aspect, the first agent is R-121,919; and the second agent is fluvoxamine. In another aspect, the first agent is R-121,919; and the second agent is sertraline. In another aspect, the first agent is R-121,919; and the second agent is desvenlafaxine. In another aspect, the first agent is R-121,919; and the second agent is duloxetine. In another aspect, the first agent is R-121,919; and the second agent is levomilnacipran. In another aspect, the first agent is R-121,919; and the second agent is milnacipran. In another aspect, the first agent is R-121,919; and the second agent is tofenacin. In another aspect, the first agent is R-121,919; and the second agent is tofenacin. In another aspect, the first agent is R-121,919; and the second agent is venlafaxine. In another aspect, the first agent is R-121,919; and the second agent is vilazodone. In another aspect, the first agent is R-121,919; and the second agent is vortioxetine. In another aspect, the first agent is R-121,919; and the second agent is bupropion. In another aspect, the first agent is R-121,919; and the second agent is agomelatine. In another aspect, the first agent is R-121,919; and the second agent is bifemelane. In another aspect, the first agent is R-121,919; and the second agent is tandospirone. In another aspect, the first agent is R-121,919; and the second agent is teniloxazine.

In another aspect, the first agent is a CRH/CRF-1 antagonist and the second agent is a beta-blocker. In another aspect, the first agent is selected from antalarmin; pexacerfont; verucerfont; LWH-234; CP-154,536; NBI-27914; and R-121,919; and the second agent is a beta-blocker. In another aspect, the first agent is selected from verucerfont; pexacerfont; LWH-234; and R-121,919; and the second agent is a beta-blocker. In another aspect, the first agent is selected from antalarmin; pexacerfont; verucerfont; LWH-234; CP-154,536; NBI-27914; and R-121,919; and the second agent is propanolol; bucindolol; carteolol; carvedilol; labetol; nadolol; oxprenolol; penbutolol; pindolol; sotalol; timolol; acebutolol; atenolol; betaxolol; bisoprolol; celiprolol; esmolol; metoprolol; nebivolol; butaxamine; ICI-118,551; or SR-59230A. In another aspect, the first agent is selected from antalarmin; pexacerfont; verucerfont; LWH-234; CP-154,536; NBI-27914; and R-121,919; and the second agent is bucindolol; metoprolol; oxprenolol; celiprolol; or nebivolol. In another aspect, the first agent is selected from verucerfont; pexacerfont; LWH-234; and R-121,919; and the second agent is propanolol; bucindolol; carteolol; carvedilol; labetol; nadolol; oxprenolol; penbutolol; pindolol; sotalol; timolol; acebutolol; atenolol; betaxolol; bisoprolol; celiprolol; esmolol; metoprolol; nebivolol; butaxamine; ICI-118,551; or SR-59230A. In another aspect, the first agent is selected from verucerfont; pexacerfont; LWH-234; and R-121,919; and the second agent is bucindolol; metoprolol; oxprenolol; celiprolol; and nebivolol.

In another aspect, the first agent is pexacerfont; and the second agent is bucindolol. In another aspect, the first agent is pexacerfont; and the second agent is metoprolol. In another aspect, the first agent is pexacerfont; and the second agent is oxprenolol. In another aspect, the first agent is pexacerfont; and the second agent is celiprolol. In another aspect, the first agent is pexacerfont; and the second agent is nebivolol.

In another aspect, the first agent is verucerfont; and the second agent is bucindolol. In another aspect, the first agent is verucerfont; and the second agent is metoprolol. In another aspect, the first agent is verucerfont; and the second agent is oxprenolol. In another aspect, the first agent is verucerfont; and the second agent is celiprolol. In another aspect, the first agent is verucerfont; and the second agent is nebivolol.

In another aspect, the first agent is LWH-234; and the second agent is bucindolol. In another aspect, the first agent is LWH-234; and the second agent is metoprolol. In another aspect, the first agent is LWH-234; and the second agent is oxprenolol. In another aspect, the first agent is LWH-234; and the second agent is celiprolol. In another aspect, the first agent is LWH-234; and the second agent is nebivolol.

In another aspect, the first agent is R-121,919; and the second agent is bucindolol. In another aspect, the first agent is R-121,919; and the second agent is metoprolol. In another aspect, the first agent is R-121,919; and the second agent is oxprenolol. In another aspect, the first agent is R-121,919; and the second agent is celiprolol. In another aspect, the first agent is R-121,919; and the second agent is nebivolol.

In another aspect, the first agent is a CRH/CRF-1 antagonist and the second agent is an antipsychotic. In another aspect, the first agent is selected from antalarmin; pexacerfont; verucerfont; LWH-234; CP-154,536; NBI-27914; and R-121,919; and the second agent is an antipsychotic. In another aspect, the first agent is selected from verucerfont; pexacerfont; LWH-234; and R-121,919; and the second agent is an antipsychotic. In another aspect, the first agent is selected from antalarmin; pexacerfont; verucerfont; LWH-234; CP-154,536; NBI-27914; and R-121,919; and the second agent is benperidol; bromperidol; droperidol; haloperidol; timiperone; diphenylbutylpiperidine; fluspirilene; penfluridol; pimozide; phenothiazines; chlorpromazine; cyamemazine; dixyrazine; fluphenazine; levomepromazine; perazine; pericyazine; perphenazine; pipotiazine; prochlorperazine; promethazine; prothipendyl; thioproperazine; trifluoperazine; chlorprothixene; clopenthixol; flupentixol; thiothixene; zuclopenthixol; clotiapine; loxapine; prothipendyl; carpipramine; clocapramine; molindone; mosapramine; sulpiride; sultopride; veralipride; amisulpride; amoxapine; arpiprazole; asenapine; cariprazine; clozapine; blonaserin; iloperidone; lurasidone; melperone; nemonapride; olanzapine; paliperidone; perospirone; quetiapine; remoxapride; risperidone; sertindole; trimipramine; ziprasidone; zotepine; brexpiprazole; ITI-007; pimavanserin; or RP5063. In another aspect, the first agent is selected from antalarmin; pexacerfont; verucerfont; LWH-234; CP-154,536; NBI-27914; and R-121,919; and the second agent is amisulpride; amoxapine; arpiprazole; asenapine; cariprazine; clozapine; blonaserin; iloperidone; lurasidone; melperone; nemonapride; olanzapine; paliperidone; perospirone; quetiapine; remoxapride; risperidone; sertindole; trimipramine; ziprasidone; or zotepine. In another aspect, the first agent is selected from verucerfont; pexacerfont; LWH-234; and R-121,919; and the second agent is benperidol; bromperidol; droperidol; haloperidol; timiperone; diphenylbutylpiperidine; fluspirilene; penfluridol; pimozide; phenothiazines; chlorpromazine; cyamemazine; dixyrazine; fluphenazine; levomepromazine; perazine; pericyazine; perphenazine; pipotiazine; prochlorperazine; promethazine; prothipendyl; thioproperazine; trifluoperazine; chlorprothixene; clopenthixol; flupentixol; thiothixene; zuclopenthixol; clotiapine; loxapine; prothipendyl; carpipramine; clocapramine; molindone; mosapramine; sulpiride; sultopride; veralipride; amisulpride; amoxapine; arpiprazole; asenapine; cariprazine; clozapine; blonaserin; iloperidone; lurasidone; melperone; nemonapride; olanzapine; paliperidone; perospirone; quetiapine; remoxapride; risperidone; sertindole; trimipramine; ziprasidone; zotepine; brexpiprazole; ITI-007; pimavanserin; or RP5063. In another aspect, the first agent is selected from verucerfont; pexacerfont; LWH-234; and R-121,919; and the second agent is amisulpride; amoxapine; arpiprazole; asenapine; cariprazine; clozapine; blonaserin; iloperidone; lurasidone; melperone; nemonapride; olanzapine; paliperidone; perospirone; quetiapine; remoxapride; risperidone; sertindole; trimipramine; ziprasidone; or zotepine.

In another aspect, the first agent is verucerfont; and the second agent is amisulpride. In another aspect, the first agent is verucerfont; and the second agent is amoxapine. In another aspect, the first agent is verucerfont; and the second agent is arpiprazole. In another aspect, the first agent is verucerfont; and the second agent is asenapine. In another aspect, the first agent is verucerfont; and the second agent is cariprazine. In another aspect, the first agent is verucerfont; and the second agent is clozapine. In another aspect, the first agent is verucerfont; and the second agent is blonaserin. In another aspect, the first agent is verucerfont; and the second agent is iloperidone. In another aspect, the first agent is verucerfont; and the second agent is lurasidone. In another aspect, the first agent is verucerfont; and the second agent is melperone. In another aspect, the first agent is verucerfont; and the second agent is nemonapride. In another aspect, the first agent is verucerfont; and the second agent is olanzapine. In another aspect, the first agent is verucerfont; and the second agent is paliperidone. In another aspect, the first agent is verucerfont; and the second agent is perospirone. In another aspect, the first agent is verucerfont; and the second agent is quetiapine. In another aspect, the first agent is verucerfont; and the second agent is remoxapride. In another aspect, the first agent is verucerfont; and the second agent is risperidone. In another aspect, the first agent is verucerfont; and the second agent is sertindole. In another aspect, the first agent is verucerfont; and the second agent is trimipramine. In another aspect, the first agent is verucerfont; and the second agent is ziprasidone. In another aspect, the first agent is verucerfont; and the second agent is zotepine.

In another aspect, the first agent is pexacerfont; and the second agent is amisulpride. In another aspect, the first agent is pexacerfont; and the second agent is amoxapine. In another aspect, the first agent is pexacerfont; and the second agent is arpiprazole. In another aspect, the first agent is pexacerfont; and the second agent is asenapine. In another aspect, the first agent is pexacerfont; and the second agent is cariprazine. In another aspect, the first agent is pexacerfont; and the second agent is clozapine. In another aspect, the first agent is pexacerfont; and the second agent is blonaserin. In another aspect, the first agent is pexacerfont; and the second agent is iloperidone. In another aspect, the first agent is pexacerfont; and the second agent is lurasidone. In another aspect, the first agent is pexacerfont; and the second agent is melperone. In another aspect, the first agent is pexacerfont; and the second agent is nemonapride. In another aspect, the first agent is pexacerfont; and the second agent is olanzapine. In another aspect, the first agent is pexacerfont; and the second agent is paliperidone. In another aspect, the first agent is pexacerfont; and the second agent is perospirone. In another aspect, the first agent is pexacerfont; and the second agent is quetiapine. In another aspect, the first agent is pexacerfont; and the second agent is remoxapride. In another aspect, the first agent is pexacerfont; and the second agent is risperidone. In another aspect, the first agent is pexacerfont; and the second agent is sertindole. In another aspect, the first agent is pexacerfont; and the second agent is trimipramine. In another aspect, the first agent is pexacerfont; and the second agent is ziprasidone. In another aspect, the first agent is pexacerfont; and the second agent is zotepine.

In another aspect, the first agent is LWH-234; and the second agent is amisulpride. In another aspect, the first agent is LWH-234; and the second agent is amoxapine. In another aspect, the first agent is LWH-234; and the second agent is arpiprazole. In another aspect, the first agent is LWH-234; and the second agent is asenapine. In another aspect, the first agent is LWH-234; and the second agent is cariprazine. In another aspect, the first agent is LWH-234; and the second agent is clozapine. In another aspect, the first agent is LWH-234; and the second agent is blonaserin. In another aspect, the first agent is LWH-234; and the second agent is iloperidone. In another aspect, the first agent is LWH-234; and the second agent is lurasidone. In another aspect, the first agent is LWH-234; and the second agent is melperone. In another aspect, the first agent is LWH-234; and the second agent is nemonapride. In another aspect, the first agent is LWH-234; and the second agent is olanzapine. In another aspect, the first agent is LWH-234; and the second agent is paliperidone. In another aspect, the first agent is LWH-234; and the second agent is perospirone. In another aspect, the first agent is LWH-234; and the second agent is quetiapine. In another aspect, the first agent is LWH-234; and the second agent is remoxapride. In another aspect, the first agent is LWH-234; and the second agent is risperidone. In another aspect, the first agent is LWH-234; and the second agent is sertindole. In another aspect, the first agent is LWH-234; and the second agent is trimipramine. In another aspect, the first agent is LWH-234; and the second agent is ziprasidone. In another aspect, the first agent is LWH-234; and the second agent is zotepine.

In another aspect, the first agent is R-121,919; and the second agent is amisulpride. In another aspect, the first agent is R-121,919; and the second agent is amoxapine. In another aspect, the first agent is R-121,919; and the second agent is arpiprazole. In another aspect, the first agent is R-121,919; and the second agent is asenapine. In another aspect, the first agent is R-121,919; and the second agent is cariprazine. In another aspect, the first agent is R-121,919; and the second agent is clozapine. In another aspect, the first agent is R-121,919; and the second agent is blonaserin. In another aspect, the first agent is R-121,919; and the second agent is iloperidone. In another aspect, the first agent is R-121,919; and the second agent is lurasidone. In another aspect, the first agent is R-121,919; and the second agent is melperone. In another aspect, the first agent is R-121,919; and the second agent is nemonapride. In another aspect, the first agent is R-121,919; and the second agent is olanzapine. In another aspect, the first agent is R-121,919; and the second agent is paliperidone. In another aspect, the first agent is R-121,919; and the second agent is perospirone. In another aspect, the first agent is R-121,919; and the second agent is quetiapine. In another aspect, the first agent is R-121,919; and the second agent is remoxapride. In another aspect, the first agent is R-121,919; and the second agent is risperidone. In another aspect, the first agent is R-121,919; and the second agent is sertindole. In another aspect, the first agent is R-121,919; and the second agent is trimipramine. In another aspect, the first agent is R-121,919; and the second agent is ziprasidone. In another aspect, the first agent is R-121,919; and the second agent is zotepine.

In another aspect, the first agent is a CRH/CRF-1 antagonist and the second agent is an alpha adrenergic agonist. In another aspect, the first agent is selected from antalarmin; pexacerfont; verucerfont; LWH-234; CP-154,536; NBI-27914; and R-121,919; and the second agent is an alpha adrenergic agonist. In another aspect, the first agent is selected from verucerfont; pexacerfont; LWH-234; and R-121,919; and the second agent is an alpha adrenergic agonist. In another aspect, the first agent is a CRH/CRF-1 antagonist and the second agent is clonidine or guanfacine. In another aspect, the first agent is selected from antalarmin; pexacerfont; verucerfont; LWH-234; CP-154,536; NBI-27914; and R-121,919; and the second agent is clonidine or guanfacine. In another aspect, the first agent is selected from verucerfont; pexacerfont; LWH-234; and R-121,919; and the second agent is clonidine or guanfacine.

In another aspect, the first agent is verucerfont; and the second agent is clonidine. In another aspect, the first agent is verucerfont; and the second agent is guanfacine.

In another aspect, the first agent is pexacerfont; and the second agent is clonidine. In another aspect, the first agent is pexacerfont; and the second agent is guanfacine.

In another aspect, the first agent is LWH-234; and the second agent is clonidine. In another aspect, the first agent is LWH-234; and the second agent is guanfacine.

In another aspect, the first agent is R-121,919; and the second agent is clonidine. In another aspect, the first agent is R-121,919; and the second agent is guanfacine.

In another aspect, the first agent is a CRH/CRF-1 antagonist and the second agent is an azapirone. In another aspect, the first agent is selected from antalarmin; pexacerfont; verucerfont; LWH-234; CP-154,536; NBI-27914; and R-121,919; and the second agent is an azapirone. In another aspect, the first agent is selected from verucerfont; pexacerfont; LWH-234; and R-121,919; and the second agent is an azapirone. In another aspect, the first agent is a CRH/CRF-1 antagonist and the second agent is buspirone or tandospirone. In another aspect, the first agent is selected from antalarmin; pexacerfont; verucerfont; LWH-234; CP-154,536; NBI-27914; and R-121,919; and the second agent is buspirone or tandospirone. In another aspect, the first agent is selected from verucerfont; pexacerfont; LWH-234; and R-121,919; and the second agent is buspirone or tandospirone.

In another aspect, the first agent is verucerfont; and the second agent is buspirone. In another aspect, the first agent is verucerfont; and the second agent is tandospirone.

In another aspect, the first agent is pexacerfont; and the second agent is buspirone. In another aspect, the first agent is pexacerfont; and the second agent is tandospirone.

In another aspect, the first agent is LWH-234; and the second agent is buspirone. In another aspect, the first agent is LWH-234; and the second agent is tandospirone.

In another aspect, the first agent is R-121,919; and the second agent is buspirone. In another aspect, the first agent is R-121,919; and the second agent is tandospirone.

In another aspect, the first agent is an ACTH antagonist and the second agent is an antidepressant. In another aspect, the first agent is selected from bromocriptine; cabergoline; somastatin analogs (e.g., octreotide, pasireotide); retinoic acid; and cyproheptadine; and the second agent is an antidepressant. In another aspect, the first agent is an ACTH antagonist and the second agent is an SSRI; SNRI; SMS; SARI; NRI; TCA; TeCA; MAOI; norepinephrine dopamine reuptake inhibitor; agomelatine; bifemelane; tandospirone; and teniloxazine. In another aspect, the first agent is selected from bromocriptine; cabergoline; somastatin analogs (e.g., octreotide, pasireotide); retinoic acid; and cyproheptadine; and the second agent is an SSRI; SNRI; SMS; bupropion; tandospirone; and teniloxazine. In another aspect, the first agent is selected from bromocriptine; cabergoline; somastatin analogs (e.g., octreotide, pasireotide); retinoic acid; and cyproheptadine; and the second agent is an S SRI; SNRI; SMS; SARI; NRI; TCA; TeCA; MAOI; norepinephrine dopamine reuptake inhibitor; agomelatine; bifemelane; tandospirone; and teniloxazine. In another aspect, the first agent is selected from bromocriptine; cabergoline; somastatin analogs (e.g., octreotide, pasireotide); retinoic acid; and cyproheptadine; and the second agent is citalopram; escitalopram; paroxetine; fluoxetine; fluvoxamine; sertraline; desvenlafaxine; duloxetine; levomilnacipran; milnacipran; tofenacin; venlafaxine; vilazodone; vortioxetine; etoperidone; nefazodone; trazodone; reboxetine; viloxazine; atromoxetine; amitriptyline; amitriptylinoxide; clomipramine; desipramine; dibenzepin; dimetacrine; dosulepin; doxepin; imipramine; lofepramine; melitracen; nitroxazepine; nortriptyline; noxiptiline; pipofezine; proptriptyline; trimipramine; opipramol; tianeptine; amoxapine; maprotiline; mianserin; mirtazapine; setiptiline; isocarboxazid; phenelzine; tranylcypromine; selegiline; metralindole; moclobemide; pirlindole; toloxatone; bupropion; agomelatine; bifemelane; tandospirone; and teniloxazine. In another aspect, the first agent is selected from bromocriptine; cabergoline; somastatin analogs (e.g., octreotide, pasireotide); retinoic acid; and cyproheptadine; and the second agent is citalopram; escitalopram; paroxetine; fluoxetine; fluvoxamine; sertraline; desvenlafaxine; duloxetine; levomilnacipran; milnacipran; tofenacin; venlafaxine; vilazodone; vortioxetine; bupropion; agomelatine; bifemelane; tandospirone; and teniloxazine.

In another aspect, the first agent is bromocriptine; and the second agent is citalopram. In another aspect, the first agent is bromocriptine; and the second agent is escitalopram. In another aspect, the first agent is bromocriptine; and the second agent is paroxetine. In another aspect, the first agent is bromocriptine; and the second agent is fluoxetine. In another aspect, the first agent is bromocriptine; and the second agent is fluvoxamine. In another aspect, the first agent is bromocriptine; and the second agent is sertraline. In another aspect, the first agent is bromocriptine; and the second agent is desvenlafaxine. In another aspect, the first agent is bromocriptine; and the second agent is duloxetine. In another aspect, the first agent is bromocriptine; and the second agent is levomilnacipran. In another aspect, the first agent is bromocriptine; and the second agent is milnacipran. In another aspect, the first agent is bromocriptine; and the second agent is tofenacin. In another aspect, the first agent is bromocriptine; and the second agent is tofenacin. In another aspect, the first agent is bromocriptine; and the second agent is venlafaxine. In another aspect, the first agent is bromocriptine; and the second agent is vilazodone. In another aspect, the first agent is bromocriptine; and the second agent is vortioxetine. In another aspect, the first agent is bromocriptine; and the second agent is bupropion. In another aspect, the first agent is bromocriptine; and the second agent is agomelatine. In another aspect, the first agent is bromocriptine; and the second agent is bifemelane. In another aspect, the first agent is bromocriptine; and the second agent is tandospirone. In another aspect, the first agent is bromocriptine; and the second agent is teniloxazine.

In another aspect, the first agent is cabergoline; and the second agent is citalopram. In another aspect, the first agent is cabergoline; and the second agent is escitalopram. In another aspect, the first agent is cabergoline; and the second agent is paroxetine. In another aspect, the first agent is cabergoline; and the second agent is fluoxetine. In another aspect, the first agent is cabergoline; and the second agent is fluvoxamine. In another aspect, the first agent is cabergoline; and the second agent is sertraline. In another aspect, the first agent is cabergoline; and the second agent is desvenlafaxine. In another aspect, the first agent is cabergoline; and the second agent is duloxetine. In another aspect, the first agent is cabergoline; and the second agent is levomilnacipran. In another aspect, the first agent is cabergoline; and the second agent is milnacipran. In another aspect, the first agent is cabergoline; and the second agent is tofenacin. In another aspect, the first agent is cabergoline; and the second agent is tofenacin. In another aspect, the first agent is cabergoline; and the second agent is venlafaxine. In another aspect, the first agent is cabergoline; and the second agent is vilazodone. In another aspect, the first agent is cabergoline; and the second agent is vortioxetine. In another aspect, the first agent is cabergoline; and the second agent is bupropion. In another aspect, the first agent is cabergoline; and the second agent is agomelatine. In another aspect, the first agent is cabergoline; and the second agent is bifemelane. In another aspect, the first agent is cabergoline; and the second agent is tandospirone. In another aspect, the first agent is cabergoline; and the second agent is teniloxazine.

In another aspect, the first agent is octreotide; and the second agent is citalopram. In another aspect, the first agent is octreotide; and the second agent is escitalopram. In another aspect, the first agent is octreotide; and the second agent is paroxetine. In another aspect, the first agent is octreotide; and the second agent is fluoxetine. In another aspect, the first agent is octreotide; and the second agent is fluvoxamine. In another aspect, the first agent is octreotide; and the second agent is sertraline. In another aspect, the first agent is octreotide; and the second agent is desvenlafaxine. In another aspect, the first agent is octreotide; and the second agent is duloxetine. In another aspect, the first agent is octreotide; and the second agent is levomilnacipran. In another aspect, the first agent is octreotide; and the second agent is milnacipran. In another aspect, the first agent is octreotide; and the second agent is tofenacin. In another aspect, the first agent is octreotide; and the second agent is tofenacin. In another aspect, the first agent is octreotide; and the second agent is venlafaxine. In another aspect, the first agent is octreotide; and the second agent is vilazodone. In another aspect, the first agent is octreotide; and the second agent is vortioxetine. In another aspect, the first agent is octreotide; and the second agent is bupropion. In another aspect, the first agent is octreotide; and the second agent is agomelatine. In another aspect, the first agent is octreotide; and the second agent is bifemelane. In another aspect, the first agent is octreotide; and the second agent is tandospirone. In another aspect, the first agent is octreotide; and the second agent is teniloxazine.

In another aspect, the first agent is pasireotide; and the second agent is citalopram. In another aspect, the first agent is pasireotide; and the second agent is escitalopram. In another aspect, the first agent is pasireotide; and the second agent is paroxetine. In another aspect, the first agent is pasireotide; and the second agent is fluoxetine. In another aspect, the first agent is pasireotide; and the second agent is fluvoxamine. In another aspect, the first agent is pasireotide; and the second agent is sertraline. In another aspect, the first agent is pasireotide; and the second agent is desvenlafaxine. In another aspect, the first agent is pasireotide; and the second agent is duloxetine. In another aspect, the first agent is pasireotide; and the second agent is levomilnacipran. In another aspect, the first agent is pasireotide; and the second agent is milnacipran. In another aspect, the first agent is pasireotide; and the second agent is tofenacin. In another aspect, the first agent is pasireotide; and the second agent is tofenacin. In another aspect, the first agent is pasireotide; and the second agent is venlafaxine. In another aspect, the first agent is pasireotide; and the second agent is vilazodone. In another aspect, the first agent is pasireotide; and the second agent is vortioxetine. In another aspect, the first agent is pasireotide; and the second agent is bupropion. In another aspect, the first agent is pasireotide; and the second agent is agomelatine. In another aspect, the first agent is pasireotide; and the second agent is bifemelane. In another aspect, the first agent is pasireotide; and the second agent is tandospirone. In another aspect, the first agent is pasireotide; and the second agent is teniloxazine.

In another aspect, the first agent is retinoic acid; and the second agent is citalopram. In another aspect, the first agent is retinoic acid; and the second agent is escitalopram. In another aspect, the first agent is retinoic acid; and the second agent is paroxetine. In another aspect, the first agent is retinoic acid; and the second agent is fluoxetine. In another aspect, the first agent is retinoic acid; and the second agent is fluvoxamine. In another aspect, the first agent is retinoic acid; and the second agent is sertraline. In another aspect, the first agent is retinoic acid; and the second agent is desvenlafaxine. In another aspect, the first agent is retinoic acid; and the second agent is duloxetine. In another aspect, the first agent is retinoic acid; and the second agent is levomilnacipran. In another aspect, the first agent is retinoic acid; and the second agent is milnacipran. In another aspect, the first agent is retinoic acid; and the second agent is tofenacin. In another aspect, the first agent is retinoic acid; and the second agent is tofenacin. In another aspect, the first agent is retinoic acid; and the second agent is venlafaxine. In another aspect, the first agent is retinoic acid; and the second agent is vilazodone. In another aspect, the first agent is retinoic acid; and the second agent is vortioxetine. In another aspect, the first agent is retinoic acid; and the second agent is bupropion. In another aspect, the first agent is retinoic acid; and the second agent is agomelatine. In another aspect, the first agent is retinoic acid; and the second agent is bifemelane. In another aspect, the first agent is retinoic acid; and the second agent is tandospirone. In another aspect, the first agent is retinoic acid; and the second agent is teniloxazine.

In another aspect, the first agent is cyproheptadine; and the second agent is citalopram. In another aspect, the first agent is cyproheptadine; and the second agent is escitalopram. In another aspect, the first agent is cyproheptadine; and the second agent is paroxetine. In another aspect, the first agent is cyproheptadine; and the second agent is fluoxetine. In another aspect, the first agent is cyproheptadine; and the second agent is fluvoxamine. In another aspect, the first agent is cyproheptadine; and the second agent is sertraline. In another aspect, the first agent is cyproheptadine; and the second agent is desvenlafaxine. In another aspect, the first agent is cyproheptadine; and the second agent is duloxetine. In another aspect, the first agent is cyproheptadine; and the second agent is levomilnacipran. In another aspect, the first agent is cyproheptadine; and the second agent is milnacipran. In another aspect, the first agent is cyproheptadine; and the second agent is tofenacin. In another aspect, the first agent is cyproheptadine; and the second agent is tofenacin. In another aspect, the first agent is cyproheptadine; and the second agent is venlafaxine. In another aspect, the first agent is cyproheptadine; and the second agent is vilazodone. In another aspect, the first agent is cyproheptadine; and the second agent is vortioxetine. In another aspect, the first agent is cyproheptadine; and the second agent is bupropion. In another aspect, the first agent is cyproheptadine; and the second agent is agomelatine. In another aspect, the first agent is cyproheptadine; and the second agent is bifemelane. In another aspect, the first agent is cyproheptadine; and the second agent is tandospirone. In another aspect, the first agent is cyproheptadine; and the second agent is teniloxazine.

In another aspect, the first agent is an ACTH antagonist and the second agent is a beta-blocker. In another aspect, the first agent is selected from bromocriptine; cabergoline; somastatin analogs (e.g., octreotide, pasireotide); retinoic acid; and cyproheptadine; and the second agent is a beta-blocker. In another aspect, the first agent is selected from bromocriptine; cabergoline; somastatin analogs (e.g., octreotide, pasireotide); retinoic acid; and cyproheptadine; and the second agent is propanolol; bucindolol; carteolol; carvedilol; labetol; nadolol; oxprenolol; penbutolol; pindolol; sotalol; timolol; acebutolol; atenolol; betaxolol; bisoprolol; celiprolol; esmolol; metoprolol; nebivolol; butaxamine; ICI-118,551; or SR-59230A. In another aspect, the first agent is selected from bromocriptine; cabergoline; somastatin analogs (e.g., octreotide, pasireotide); retinoic acid; and cyproheptadine; and the second agent is bucindolol; metoprolol; oxprenolol; celiprolol; or nebivolol.

In another aspect, the first agent is bromocriptine; and the second agent is bucindolol. In another aspect, the first agent is bromocriptine; and the second agent is metoprolol. In another aspect, the first agent is bromocriptine; and the second agent is oxprenolol. In another aspect, the first agent is bromocriptine; and the second agent is celiprolol. In another aspect, the first agent is bromocriptine; and the second agent is nebivolol.

In another aspect, the first agent is cabergoline; and the second agent is bucindolol. In another aspect, the first agent is cabergoline; and the second agent is metoprolol. In another aspect, the first agent is cabergoline; and the second agent is oxprenolol. In another aspect, the first agent is cabergoline; and the second agent is celiprolol. In another aspect, the first agent is cabergoline; and the second agent is nebivolol.

In another aspect, the first agent is octreotide; and the second agent is bucindolol. In another aspect, the first agent is octreotide; and the second agent is metoprolol. In another aspect, the first agent is octreotide; and the second agent is oxprenolol. In another aspect, the first agent is octreotide; and the second agent is celiprolol. In another aspect, the first agent is octreotide; and the second agent is nebivolol.

In another aspect, the first agent is pasireotide; and the second agent is bucindolol. In another aspect, the first agent is pasireotide; and the second agent is metoprolol. In another aspect, the first agent is pasireotide; and the second agent is oxprenolol. In another aspect, the first agent is pasireotide; and the second agent is celiprolol. In another aspect, the first agent is pasireotide; and the second agent is nebivolol.

In another aspect, the first agent is retinoic acid; and the second agent is bucindolol. In another aspect, the first agent is retinoic acid; and the second agent is metoprolol. In another aspect, the first agent is retinoic acid; and the second agent is oxprenolol. In another aspect, the first agent is retinoic acid; and the second agent is celiprolol. In another aspect, the first agent is retinoic acid; and the second agent is nebivolol.

In another aspect, the first agent is cyproheptadine; and the second agent is bucindolol. In another aspect, the first agent is cyproheptadine; and the second agent is metoprolol. In another aspect, the first agent is cyproheptadine; and the second agent is oxprenolol. In another aspect, the first agent is cyproheptadine; and the second agent is celiprolol. In another aspect, the first agent is cyproheptadine; and the second agent is nebivolol.

In another aspect, the first agent is an ACTH antagonist and the second agent is an antipsychotic. In another aspect, the first agent is selected from bromocriptine; cabergoline; somastatin analogs (e.g., octreotide, pasireotide); retinoic acid; and cyproheptadine; and the second agent is an antipsychotic. In another aspect, the first agent is selected from bromocriptine; cabergoline; somastatin analogs (e.g., octreotide, pasireotide); retinoic acid; and cyproheptadine; and the second agent is benperidol; bromperidol; droperidol; haloperidol; timiperone; diphenylbutylpiperidine; fluspirilene; penfluridol; pimozide; phenothiazines; chlorpromazine; cyamemazine; dixyrazine; fluphenazine; levomepromazine; perazine; pericyazine; perphenazine; pipotiazine; prochlorperazine; promethazine; prothipendyl; thioproperazine; trifluoperazine; chlorprothixene; clopenthixol; flupentixol; thiothixene; zuclopenthixol; clotiapine; loxapine; prothipendyl; carpipramine; clocapramine; molindone; mosapramine; sulpiride; sultopride; veralipride; amisulpride; amoxapine; arpiprazole; asenapine; cariprazine; clozapine; blonaserin; iloperidone; lurasidone; melperone; nemonapride; olanzapine; paliperidone; perospirone; quetiapine; remoxapride; risperidone; sertindole; trimipramine; ziprasidone; zotepine; brexpiprazole; ITI-007; pimavanserin; or RP5063. In another aspect, the first agent is selected from bromocriptine; cabergoline; somastatin analogs (e.g., octreotide, pasireotide); retinoic acid; and cyproheptadine; and the second agent is amisulpride; amoxapine; arpiprazole; asenapine; cariprazine; clozapine; blonaserin; iloperidone; lurasidone; melperone; nemonapride; olanzapine; paliperidone; perospirone; quetiapine; remoxapride; risperidone; sertindole; trimipramine; ziprasidone; or zotepine.

In another aspect, the first agent is bromocriptine; and the second agent is amisulpride. In another aspect, the first agent is bromocriptine; and the second agent is amoxapine. In another aspect, the first agent is bromocriptine; and the second agent is arpiprazole. In another aspect, the first agent is bromocriptine; and the second agent is asenapine. In another aspect, the first agent is bromocriptine; and the second agent is cariprazine. In another aspect, the first agent is bromocriptine; and the second agent is clozapine. In another aspect, the first agent is bromocriptine; and the second agent is blonaserin. In another aspect, the first agent is bromocriptine; and the second agent is iloperidone. In another aspect, the first agent is bromocriptine; and the second agent is lurasidone. In another aspect, the first agent is bromocriptine; and the second agent is melperone. In another aspect, the first agent is bromocriptine; and the second agent is nemonapride. In another aspect, the first agent is bromocriptine; and the second agent is olanzapine. In another aspect, the first agent is bromocriptine; and the second agent is paliperidone. In another aspect, the first agent is bromocriptine; and the second agent is perospirone. In another aspect, the first agent is bromocriptine; and the second agent is quetiapine. In another aspect, the first agent is bromocriptine; and the second agent is remoxapride. In another aspect, the first agent is bromocriptine; and the second agent is risperidone. In another aspect, the first agent is bromocriptine; and the second agent is sertindole. In another aspect, the first agent is bromocriptine; and the second agent is trimipramine. In another aspect, the first agent is bromocriptine; and the second agent is ziprasidone. In another aspect, the first agent is bromocriptine; and the second agent is zotepine.

In another aspect, the first agent is cabergoline; and the second agent is amisulpride. In another aspect, the first agent is cabergoline; and the second agent is amoxapine. n another aspect, the first agent is cabergoline; and the second agent is arpiprazole. In another aspect, the first agent is cabergoline; and the second agent is asenapine. In another aspect, the first agent is cabergoline; and the second agent is cariprazine. In another aspect, the first agent is cabergoline; and the second agent is clozapine. In another aspect, the first agent is bromocriptine; and the second agent is blonaserin. In another aspect, the first agent is cabergoline; and the second agent is iloperidone. In another aspect, the first agent is cabergoline; and the second agent is lurasidone. In another aspect, the first agent is cabergoline; and the second agent is melperone. In another aspect, the first agent is cabergoline; and the second agent is nemonapride. In another aspect, the first agent is cabergoline; and the second agent is olanzapine. In another aspect, the first agent is cabergoline; and the second agent is paliperidone. In another aspect, the first agent is cabergoline; and the second agent is perospirone. In another aspect, the first agent is cabergoline; and the second agent is quetiapine. In another aspect, the first agent is cabergoline; and the second agent is remoxapride. In another aspect, the first agent is cabergoline; and the second agent is risperidone. In another aspect, the first agent is cabergoline; and the second agent is sertindole. In another aspect, the first agent is cabergoline; and the second agent is trimipramine. In another aspect, the first agent is cabergoline; and the second agent is ziprasidone. In another aspect, the first agent is cabergoline; and the second agent is zotepine.

In another aspect, the first agent is octreotide; and the second agent is amisulpride. In another aspect, the first agent is octreotide; and the second agent is amoxapine. In another aspect, the first agent is octreotide; and the second agent is arpiprazole. In another aspect, the first agent is octreotide; and the second agent is asenapine. In another aspect, the first agent is octreotide; and the second agent is cariprazine. In another aspect, the first agent is octreotide; and the second agent is clozapine. In another aspect, the first agent is octreotide; and the second agent is blonaserin. In another aspect, the first agent is octreotide; and the second agent is iloperidone. In another aspect, the first agent is octreotide; and the second agent is lurasidone. In another aspect, the first agent is octreotide; and the second agent is melperone. In another aspect, the first agent is octreotide; and the second agent is nemonapride. In another aspect, the first agent is octreotide; and the second agent is olanzapine. In another aspect, the first agent is octreotide; and the second agent is paliperidone. In another aspect, the first agent is octreotide; and the second agent is perospirone. In another aspect, the first agent is octreotide; and the second agent is quetiapine. In another aspect, the first agent is octreotide; and the second agent is remoxapride. In another aspect, the first agent is octreotide; and the second agent is risperidone. In another aspect, the first agent is octreotide; and the second agent is sertindole. In another aspect, the first agent is octreotide; and the second agent is trimipramine. In another aspect, the first agent is octreotide; and the second agent is ziprasidone. In another aspect, the first agent is octreotide; and the second agent is zotepine.

In another aspect, the first agent is pasireotide; and the second agent is amisulpride. In another aspect, the first agent is pasireotide; and the second agent is amoxapine. In another aspect, the first agent is pasireotide; and the second agent is arpiprazole. In another aspect, the first agent is pasireotide; and the second agent is asenapine. In another aspect, the first agent is pasireotide; and the second agent is cariprazine. In another aspect, the first agent is pasireotide; and the second agent is clozapine. In another aspect, the first agent is pasireotide; and the second agent is blonaserin. In another aspect, the first agent is pasireotide; and the second agent is iloperidone. In another aspect, the first agent is pasireotide; and the second agent is lurasidone. In another aspect, the first agent is pasireotide; and the second agent is melperone. In another aspect, the first agent is pasireotide; and the second agent is nemonapride. In another aspect, the first agent is pasireotide; and the second agent is olanzapine. In another aspect, the first agent is pasireotide; and the second agent is paliperidone. In another aspect, the first agent is pasireotide; and the second agent is perospirone. In another aspect, the first agent is pasireotide; and the second agent is quetiapine. In another aspect, the first agent is pasireotide; and the second agent is remoxapride. In another aspect, the first agent is pasireotide; and the second agent is risperidone. In another aspect, the first agent is pasireotide; and the second agent is sertindole. In another aspect, the first agent is pasireotide; and the second agent is trimipramine. In another aspect, the first agent is pasireotide; and the second agent is ziprasidone. In another aspect, the first agent is pasireotide; and the second agent is zotepine.

In another aspect, the first agent is retinoic acid; and the second agent is amisulpride. In another aspect, the first agent is retinoic acid; and the second agent is amoxapine. In another aspect, the first agent is retinoic acid; and the second agent is arpiprazole. In another aspect, the first agent is retinoic acid; and the second agent is asenapine. In another aspect, the first agent is retinoic acid; and the second agent is cariprazine. In another aspect, the first agent is retinoic acid; and the second agent is clozapine. In another aspect, the first agent is retinoic acid; and the second agent is blonaserin. In another aspect, the first agent is retinoic acid; and the second agent is iloperidone. In another aspect, the first agent is retinoic acid; and the second agent is lurasidone. In another aspect, the first agent is retinoic acid; and the second agent is melperone. In another aspect, the first agent is retinoic acid; and the second agent is nemonapride. In another aspect, the first agent is retinoic acid; and the second agent is olanzapine. In another aspect, the first agent is retinoic acid; and the second agent is paliperidone. In another aspect, the first agent is retinoic acid; and the second agent is perospirone. In another aspect, the first agent is retinoic acid; and the second agent is quetiapine. In another aspect, the first agent is retinoic acid; and the second agent is remoxapride. In another aspect, the first agent is retinoic acid; and the second agent is risperidone. In another aspect, the first agent is retinoic acid; and the second agent is sertindole. In another aspect, the first agent is retinoic acid; and the second agent is trimipramine. In another aspect, the first agent is retinoic acid; and the second agent is ziprasidone. In another aspect, the first agent is retinoic acid; and the second agent is zotepine.

In another aspect, the first agent is cyproheptadine; and the second agent is amisulpride. In another aspect, the first agent is cyproheptadine; and the second agent is amoxapine. In another aspect, the first agent is cyproheptadine; and the second agent is arpiprazole. In another aspect, the first agent is cyproheptadine; and the second agent is asenapine. In another aspect, the first agent is cyproheptadine; and the second agent is cariprazine. In another aspect, the first agent is cyproheptadine; and the second agent is clozapine. In another aspect, the first agent is pasir cyproheptadine eotide; and the second agent is blonaserin. In another aspect, the first agent is cyproheptadine; and the second agent is iloperidone. In another aspect, the first agent is cyproheptadine; and the second agent is lurasidone. In another aspect, the first agent is cyproheptadine; and the second agent is melperone. In another aspect, the first agent is cyproheptadine; and the second agent is nemonapride. In another aspect, the first agent is cyproheptadine; and the second agent is olanzapine. In another aspect, the first agent is cyproheptadine; and the second agent is paliperidone. In another aspect, the first agent is cyproheptadine; and the second agent is perospirone. In another aspect, the first agent is cyproheptadine; and the second agent is quetiapine. In another aspect, the first agent is cyproheptadine; and the second agent is remoxapride. In another aspect, the first agent is cyproheptadine; and the second agent is risperidone. In another aspect, the first agent is cyproheptadine; and the second agent is sertindole. In another aspect, the first agent is cyproheptadine; and the second agent is trimipramine. In another aspect, the first agent is cyproheptadine; and the second agent is ziprasidone. In another aspect, the first agent is cyproheptadine; and the second agent is zotepine.

In another aspect, the first agent is an ACTH antagonist and the second agent is an alpha adrenergic agonist. In another aspect, the first agent is selected from bromocriptine; cabergoline; somastatin analogs (e.g., octreotide, pasireotide); retinoic acid; and cyproheptadine; and the second agent is an alpha adrenergic agonist. In another aspect, the first agent is an ACTH antagonist and the second agent is clonidine or guanfacine. In another aspect, the first agent is selected from bromocriptine; cabergoline; somastatin analogs (e.g., octreotide, pasireotide); retinoic acid; and cyproheptadine; and the second agent is clonidine or guanfacine.

In another aspect, the first agent is bromocriptine; and the second agent is clonidine. In another aspect, the first agent is bromocriptine; and the second agent is guanfacine.

In another aspect, the first agent is cabergoline; and the second agent is clonidine. In another aspect, the first agent is cabergoline; and the second agent is guanfacine.

In another aspect, the first agent is octreotide; and the second agent is clonidine. In another aspect, the first agent is octreotide; and the second agent is guanfacine.

In another aspect, the first agent is pasireotide; and the second agent is clonidine. In another aspect, the first agent is pasireotide; and the second agent is guanfacine.

In another aspect, the first agent is retinoic acid; and the second agent is clonidine. In another aspect, the first agent is retinoic acid; and the second agent is guanfacine.

In another aspect, the first agent is cyproheptadine; and the second agent is clonidine. In another aspect, the first agent is cyproheptadine; and the second agent is guanfacine.

In another aspect, the first agent is an ACTH antagonist and the second agent is an azapirone. In another aspect, the first agent is selected from bromocriptine; cabergoline; somastatin analogs (e.g., octreotide, pasireotide); retinoic acid; and cyproheptadine; and the second agent is an azapirone. In another aspect, the first agent is an ACTH antagonist and the second agent is buspirone or tandospirone. In another aspect, the first agent is selected from bromocriptine; cabergoline; somastatin analogs (e.g., octreotide, pasireotide); retinoic acid; and cyproheptadine; and the second agent is buspirone or tandospirone.

In another aspect, the first agent is bromocriptine; and the second agent is buspirone. In another aspect, the first agent is bromocriptine; and the second agent is tandospirone.

In another aspect, the first agent is cabergoline; and the second agent is buspirone. In another aspect, the first agent is cabergoline; and the second agent is tandospirone.

In another aspect, the first agent is octreotide; and the second agent is buspirone. In another aspect, the first agent is octreotide; and the second agent is tandospirone.

In another aspect, the first agent is pasireotide; and the second agent is buspirone. In another aspect, the first agent is pasireotide; and the second agent is tandospirone.

In another aspect, the first agent is retinoic acid; and the second agent is buspirone. In another aspect, the first agent is retinoic acid; and the second agent is tandospirone.

In another aspect, the first agent is cyproheptadine; and the second agent is buspirone. In another aspect, the first agent is cyproheptadine; and the second agent is tandospirone.

In another aspect, the first agent is a cortisol inhibitor and the second agent is an antidepressant. In another aspect, the first agent is selected from metyrapone; ketoconazole; mitotane; aminoglutethimide; etomidate; mifepristone; cytadren; and fluconazole; and the second agent is an antidepressant. In another aspect, the first agent is selected from mifepristone; cytadren; and fluconazole; and the second agent is an antidepressant. In another aspect, the first agent is a cortisol inhibitor and the second agent is an SSRI; SNRI; SMS; SARI; NRI; TCA; TeCA; MAOI; norepinephrine dopamine reuptake inhibitor; agomelatine; bifemelane; tandospirone; and teniloxazine. In another aspect, the first agent is selected from metyrapone; ketoconazole; mitotane; aminoglutethimide; etomidate; mifepristone; cytadren; and fluconazole; and the second agent is an SSRI; SNRI; SMS; bupropion; tandospirone; and teniloxazine. In another aspect, the first agent is selected from metyrapone; ketoconazole; mitotane; aminoglutethimide; etomidate; mifepristone; cytadren; and fluconazole; and the second agent is an SSRI; SNRI; SMS; SARI; NRI; TCA; TeCA; MAOI; norepinephrine dopamine reuptake inhibitor; agomelatine; bifemelane; tandospirone; and teniloxazine. In another aspect, the first agent is selected from metyrapone; ketoconazole; mitotane; aminoglutethimide; etomidate; mifepristone; cytadren; and fluconazole; and the second agent is an SSRI; SNRI; SMS; bupropion; tandospirone; and teniloxazine. In another aspect, the first agent is selected from mifepristone; cytadren; and fluconazole; and the second agent is an SSRI; SNRI; SMS; SARI; NRI; TCA; TeCA; MAOI; norepinephrine dopamine reuptake inhibitor; agomelatine; bifemelane; tandospirone; and teniloxazine. In another aspect, the first agent is selected from mifepristone; cytadren; and fluconazole; and the second agent is an SSRI; SNRI; SMS; bupropion; tandospirone; and teniloxazine. In another aspect, the first agent is selected from metyrapone; ketoconazole; mitotane; aminoglutethimide; etomidate; mifepristone; cytadren; and fluconazole; and the second agent is citalopram; escitalopram; paroxetine; fluoxetine; fluvoxamine; sertraline; desvenlafaxine; duloxetine; levomilnacipran; milnacipran; tofenacin; venlafaxine; vilazodone; vortioxetine; etoperidone; nefazodone; trazodone; reboxetine; viloxazine; atromoxetine; amitriptyline; amitriptylinoxide; clomipramine; desipramine; dibenzepin; dimetacrine; dosulepin; doxepin; imipramine; lofepramine; melitracen; nitroxazepine; nortriptyline; noxiptiline; pipofezine; proptriptyline; trimipramine; opipramol; tianeptine; amoxapine; maprotiline; mianserin; mirtazapine; setiptiline; isocarboxazid; phenelzine; tranylcypromine; selegiline; metralindole; moclobemide; pirlindole; toloxatone; bupropion; agomelatine; bifemelane; tandospirone; and teniloxazine. In another aspect, the first agent is selected from mifepristone; cytadren; and fluconazole; and the second agent is citalopram; escitalopram; paroxetine; fluoxetine; fluvoxamine; sertraline; desvenlafaxine; duloxetine; levomilnacipran; milnacipran; tofenacin; venlafaxine; vilazodone; vortioxetine; etoperidone; nefazodone; trazodone; reboxetine; viloxazine; atromoxetine; amitriptyline; amitriptylinoxide; clomipramine; desipramine; dibenzepin; dimetacrine; dosulepin; doxepin; imipramine; lofepramine; melitracen; nitroxazepine; nortriptyline; noxiptiline; pipofezine; proptriptyline; trimipramine; opipramol; tianeptine; amoxapine; maprotiline; mianserin; mirtazapine; setiptiline; isocarboxazid; phenelzine; tranylcypromine; selegiline; metralindole; moclobemide; pirlindole; toloxatone; bupropion; agomelatine; bifemelane; tandospirone; and teniloxazine. In another aspect, the first agent is selected from metyrapone; ketoconazole; mitotane; aminoglutethimide; etomidate; mifepristone; cytadren; and fluconazole; and the second agent is citalopram; escitalopram; paroxetine; fluoxetine; fluvoxamine; sertraline; desvenlafaxine; duloxetine; levomilnacipran; milnacipran; tofenacin; venlafaxine; vilazodone; vortioxetine; bupropion; agomelatine; bifemelane; tandospirone; and teniloxazine. In another aspect, the first agent is selected from mifepristone; cytadren; and fluconazole; and the second agent is citalopram; escitalopram; paroxetine; fluoxetine; fluvoxamine; sertraline; desvenlafaxine; duloxetine; levomilnacipran; milnacipran; tofenacin; venlafaxine; vilazodone; vortioxetine; bupropion; agomelatine; bifemelane; tandospirone; and teniloxazine.

In another aspect, the first agent is mifepristone; and the second agent is citalopram. In another aspect, the first agent is mifepristone; and the second agent is escitalopram. In another aspect, the first agent is mifepristone; and the second agent is paroxetine. In another aspect, the first agent is mifepristone; and the second agent is fluoxetine. In another aspect, the first agent is mifepristone; and the second agent is fluvoxamine. In another aspect, the first agent is mifepristone; and the second agent is sertraline. In another aspect, the first agent is mifepristone; and the second agent is desvenlafaxine. In another aspect, the first agent is mifepristone; and the second agent is duloxetine. In another aspect, the first agent is mifepristone; and the second agent is levomilnacipran. In another aspect, the first agent is mifepristone; and the second agent is milnacipran. In another aspect, the first agent is mifepristone; and the second agent is tofenacin. In another aspect, the first agent is mifepristone; and the second agent is tofenacin. In another aspect, the first agent is mifepristone; and the second agent is venlafaxine. In another aspect, the first agent is mifepristone; and the second agent is vilazodone. In another aspect, the first agent is mifepristone; and the second agent is vortioxetine. In another aspect, the first agent is mifepristone; and the second agent is bupropion. In another aspect, the first agent is mifepristone; and the second agent is agomelatine. In another aspect, the first agent is mifepristone; and the second agent is bifemelane. In another aspect, the first agent is mifepristone; and the second agent is tandospirone. In another aspect, the first agent is mifepristone; and the second agent is teniloxazine.

In another aspect, the first agent is cytadren; and the second agent is citalopram. In another aspect, the first agent is cytadren; and the second agent is escitalopram. In another aspect, the first agent is cytadren; and the second agent is paroxetine. In another aspect, the first agent is cytadren; and the second agent is fluoxetine. In another aspect, the first agent is cytadren; and the second agent is fluvoxamine. In another aspect, the first agent is cytadren; and the second agent is sertraline. In another aspect, the first agent is cytadren; and the second agent is desvenlafaxine. In another aspect, the first agent is cytadren; and the second agent is duloxetine. In another aspect, the first agent is cytadren; and the second agent is levomilnacipran. In another aspect, the first agent is cytadren; and the second agent is milnacipran. In another aspect, the first agent is cytadren; and the second agent is tofenacin. In another aspect, the first agent is cytadren; and the second agent is tofenacin. In another aspect, the first agent is cytadren; and the second agent is venlafaxine. In another aspect, the first agent is cytadren; and the second agent is vilazodone. In another aspect, the first agent is cytadren; and the second agent is vortioxetine. In another aspect, the first agent is cytadren; and the second agent is bupropion. In another aspect, the first agent is cytadren; and the second agent is agomelatine. In another aspect, the first agent is cytadren; and the second agent is bifemelane. In another aspect, the first agent is cytadren; and the second agent is tandospirone. In another aspect, the first agent is cytadren; and the second agent is teniloxazine.

In another aspect, the first agent is fluconazole; and the second agent is citalopram. In another aspect, the first agent is fluconazole; and the second agent is escitalopram. In another aspect, the first agent is fluconazole; and the second agent is paroxetine. In another aspect, the first agent is fluconazole; and the second agent is fluoxetine. In another aspect, the first agent is fluconazole; and the second agent is fluvoxamine. In another aspect, the first agent is fluconazole; and the second agent is sertraline. In another aspect, the first agent is fluconazole; and the second agent is desvenlafaxine. In another aspect, the first agent is fluconazole; and the second agent is duloxetine. In another aspect, the first agent is fluconazole; and the second agent is levomilnacipran. In another aspect, the first agent is fluconazole; and the second agent is milnacipran. In another aspect, the first agent is fluconazole; and the second agent is tofenacin. In another aspect, the first agent is fluconazole; and the second agent is tofenacin. In another aspect, the first agent is fluconazole; and the second agent is venlafaxine. In another aspect, the first agent is fluconazole; and the second agent is vilazodone. In another aspect, the first agent is fluconazole; and the second agent is vortioxetine. In another aspect, the first agent is fluconazole; and the second agent is bupropion. In another aspect, the first agent is fluconazole; and the second agent is agomelatine. In another aspect, the first agent is fluconazole; and the second agent is bifemelane. In another aspect, the first agent is fluconazole; and the second agent is tandospirone. In another aspect, the first agent is fluconazole; and the second agent is teniloxazine.

In another aspect, the first agent is a cortisol inhibitor and the second agent is a beta-blocker. In another aspect, the first agent is selected from metyrapone; ketoconazole; mitotane; aminoglutethimide; etomidate; mifepristone; cytadren; and fluconazole; and the second agent is a beta-blocker. In another aspect, the first agent is selected from verucerfont; LWH-234; and R-121,919; and the second agent is a beta-blocker. In another aspect, the first agent is selected from metyrapone; ketoconazole; mitotane; aminoglutethimide; etomidate; mifepristone; cytadren; and fluconazole; and the second agent is propanolol; bucindolol; carteolol; carvedilol; labetol; nadolol; oxprenolol; penbutolol; pindolol; sotalol; timolol; acebutolol; atenolol; betaxolol; bisoprolol; celiprolol; esmolol; metoprolol; nebivolol; butaxamine; ICI-118,551; or SR-59230A. In another aspect, the first agent is selected from metyrapone; ketoconazole; mitotane; aminoglutethimide; etomidate; mifepristone; cytadren; and fluconazole; and the second agent is bucindolol; metoprolol; oxprenolol; celiprolol; or nebivolol. In another aspect, the first agent is selected from mifepristone; cytadren; and fluconazole; and the second agent is propanolol; bucindolol; carteolol; carvedilol; labetol; nadolol; oxprenolol; penbutolol; pindolol; sotalol; timolol; acebutolol; atenolol; betaxolol; bisoprolol; celiprolol; esmolol; metoprolol; nebivolol; butaxamine; ICI-118,551; or SR-59230A. In another aspect, the first agent is selected from mifepristone; cytadren; and fluconazole; and the second agent is bucindolol; metoprolol; oxprenolol; celiprolol; or nebivolol.

In another aspect, the first agent is mifepristone; and the second agent is bucindolol. In another aspect, the first agent is mifepristone; and the second agent is metoprolol. In another aspect, the first agent is mifepristone; and the second agent is oxprenolol. In another aspect, the first agent is mifepristone; and the second agent is celiprolol. In another aspect, the first agent is mifepristone; and the second agent is nebivolol.

In another aspect, the first agent is cytadren; and the second agent is bucindolol. In another aspect, the first agent is cytadren; and the second agent is metoprolol. In another aspect, the first agent is cytadren; and the second agent is oxprenolol. In another aspect, the first agent is cytadren; and the second agent is celiprolol. In another aspect, the first agent is cytadren; and the second agent is nebivolol.

In another aspect, the first agent is fluconazole; and the second agent is bucindolol. In another aspect, the first agent is fluconazole; and the second agent is metoprolol. In another aspect, the first agent is fluconazole; and the second agent is oxprenolol. In another aspect, the first agent is fluconazole; and the second agent is celiprolol. In another aspect, the first agent is fluconazole; and the second agent is nebivolol.

In another aspect, the first agent is a cortisol inhibitor and the second agent is an antipsychotic. In another aspect, the first agent is selected from metyrapone; ketoconazole; mitotane; aminoglutethimide; etomidate; mifepristone; cytadren; and fluconazole; and the second agent is an antipsychotic. In another aspect, the first agent is selected from mifepristone; cytadren; and fluconazole; and the second agent is an antipsychotic. In another aspect, the first agent is selected from metyrapone; ketoconazole; mitotane; aminoglutethimide; etomidate; mifepristone; cytadren; and fluconazole; and the second agent is benperidol; bromperidol; droperidol; haloperidol; timiperone; diphenylbutylpiperidine; fluspirilene; penfluridol; pimozide; phenothiazines; chlorpromazine; cyamemazine; dixyrazine; fluphenazine; levomepromazine; perazine; pericyazine; perphenazine; pipotiazine; prochlorperazine; promethazine; prothipendyl; thioproperazine; trifluoperazine; chlorprothixene; clopenthixol; flupentixol; thiothixene; zuclopenthixol; clotiapine; loxapine; prothipendyl; carpipramine; clocapramine; molindone; mosapramine; sulpiride; sultopride; veralipride; amisulpride; amoxapine; arpiprazole; asenapine; cariprazine; clozapine; blonaserin; iloperidone; lurasidone; melperone; nemonapride; olanzapine; paliperidone; perospirone; quetiapine; remoxapride; risperidone; sertindole; trimipramine; ziprasidone; zotepine; brexpiprazole; ITI-007; pimavanserin; or RP5063. In another aspect, the first agent is selected from metyrapone; ketoconazole; mitotane; aminoglutethimide; etomidate; mifepristone; cytadren; and fluconazole; and the second agent is amisulpride; amoxapine; arpiprazole; asenapine; cariprazine; clozapine; blonaserin; iloperidone; lurasidone; melperone; nemonapride; olanzapine; paliperidone; perospirone; quetiapine; remoxapride; risperidone; sertindole; trimipramine; ziprasidone; or zotepine. In another aspect, the first agent is selected from mifepristone; cytadren; and fluconazole; and the second agent is benperidol; bromperidol; droperidol; haloperidol; timiperone; diphenylbutylpiperidine; fluspirilene; penfluridol; pimozide; phenothiazines; chlorpromazine; cyamemazine; dixyrazine; fluphenazine; levomepromazine; perazine; pericyazine; perphenazine; pipotiazine; prochlorperazine; promethazine; prothipendyl; thioproperazine; trifluoperazine; chlorprothixene; clopenthixol; flupentixol; thiothixene; zuclopenthixol; clotiapine; loxapine; prothipendyl; carpipramine; clocapramine; molindone; mosapramine; sulpiride; sultopride; veralipride; amisulpride; amoxapine; arpiprazole; asenapine; cariprazine; clozapine; blonaserin; iloperidone; lurasidone; melperone; nemonapride; olanzapine; paliperidone; perospirone; quetiapine; remoxapride; risperidone; sertindole; trimipramine; ziprasidone; zotepine; brexpiprazole; ITI-007; pimavanserin; or RP5063. In another aspect, the first agent is selected from mifepristone; cytadren; and fluconazole; and the second agent is amisulpride; amoxapine; arpiprazole; asenapine; cariprazine; clozapine; blonaserin; iloperidone; lurasidone; melperone; nemonapride; olanzapine; paliperidone; perospirone; quetiapine; remoxapride; risperidone; sertindole; trimipramine; ziprasidone; or zotepine.

In another aspect, the first agent is mifepristone; and the second agent is amisulpride. In another aspect, the first agent is mifepristone; and the second agent is amoxapine. In another aspect, the first agent is mifepristone; and the second agent is arpiprazole. In another aspect, the first agent is mifepristone; and the second agent is asenapine. In another aspect, the first agent is mifepristone; and the second agent is cariprazine. In another aspect, the first agent is mifepristone; and the second agent is clozapine. In another aspect, the first agent is mifepristone; and the second agent is blonaserin. In another aspect, the first agent is mifepristone; and the second agent is iloperidone. In another aspect, the first agent is mifepristone; and the second agent is lurasidone. In another aspect, the first agent is mifepristone; and the second agent is melperone. In another aspect, the first agent is mifepristone; and the second agent is nemonapride. In another aspect, the first agent is mifepristone; and the second agent is olanzapine. In another aspect, the first agent is mifepristone; and the second agent is paliperidone. In another aspect, the first agent is mifepristone; and the second agent is perospirone. In another aspect, the first agent is mifepristone; and the second agent is quetiapine. In another aspect, the first agent is mifepristone; and the second agent is remoxapride. In another aspect, the first agent is mifepristone; and the second agent is risperidone. In another aspect, the first agent is mifepristone; and the second agent is sertindole. In another aspect, the first agent is mifepristone; and the second agent is trimipramine. In another aspect, the first agent is mifepristone; and the second agent is ziprasidone. In another aspect, the first agent is mifepristone; and the second agent is zotepine.

In another aspect, the first agent is cytadren; and the second agent is amisulpride. In another aspect, the first agent is cytadren; and the second agent is amoxapine. In another aspect, the first agent is cytadren; and the second agent is arpiprazole. In another aspect, the first agent is cytadren; and the second agent is asenapine. In another aspect, the first agent is cytadren; and the second agent is cariprazine. In another aspect, the first agent is cytadren; and the second agent is clozapine. In another aspect, the first agent is cytadren; and the second agent is blonaserin. In another aspect, the first agent is cytadren; and the second agent is iloperidone. In another aspect, the first agent is cytadren; and the second agent is lurasidone. In another aspect, the first agent is cytadren; and the second agent is melperone. In another aspect, the first agent is cytadren; and the second agent is nemonapride. In another aspect, the first agent is cytadren; and the second agent is olanzapine. In another aspect, the first agent is cytadren; and the second agent is paliperidone. In another aspect, the first agent is cytadren; and the second agent is perospirone. In another aspect, the first agent is cytadren; and the second agent is quetiapine. In another aspect, the first agent is cytadren; and the second agent is remoxapride. In another aspect, the first agent is cytadren; and the second agent is risperidone. In another aspect, the first agent is cytadren; and the second agent is sertindole. In another aspect, the first agent is cytadren; and the second agent is trimipramine. In another aspect, the first agent is cytadren; and the second agent is ziprasidone. In another aspect, the first agent is cytadren; and the second agent is zotepine.

In another aspect, the first agent is fluconazole; and the second agent is amisulpride. In another aspect, the first agent is fluconazole; and the second agent is amoxapine. In another aspect, the first agent is fluconazole; and the second agent is arpiprazole. In another aspect, the first agent is fluconazole; and the second agent is asenapine. In another aspect, the first agent is fluconazole; and the second agent is cariprazine. In another aspect, the first agent is fluconazole; and the second agent is clozapine. In another aspect, the first agent is fluconazole; and the second agent is blonaserin. In another aspect, the first agent is fluconazole; and the second agent is iloperidone. In another aspect, the first agent is fluconazole; and the second agent is lurasidone. In another aspect, the first agent is fluconazole; and the second agent is melperone. In another aspect, the first agent is fluconazole; and the second agent is nemonapride. In another aspect, the first agent is fluconazole; and the second agent is olanzapine. In another aspect, the first agent is fluconazole; and the second agent is paliperidone. In another aspect, the first agent is fluconazole; and the second agent is perospirone. In another aspect, the first agent is fluconazole; and the second agent is quetiapine. In another aspect, the first agent is fluconazole; and the second agent is remoxapride. In another aspect, the first agent is fluconazole; and the second agent is risperidone. In another aspect, the first agent is fluconazole; and the second agent is sertindole. In another aspect, the first agent is fluconazole; and the second agent is trimipramine. In another aspect, the first agent is fluconazole; and the second agent is ziprasidone. In another aspect, the first agent is fluconazole; and the second agent is zotepine.

In another aspect, the first agent is a cortisol inhibitor and the second agent is an alpha adrenergic agonist. In another aspect, the first agent is selected from metyrapone; ketoconazole; mitotane; aminoglutethimide; etomidate; mifepristone; cytadren; and fluconazole; and the second agent is an alpha adrenergic agonist. In another aspect, the first agent is selected from mifepristone; cytadren; and fluconazole; and the second agent is an alpha adrenergic agonist. In another aspect, the first agent is a cortisol inhibitor and the second agent is clonidine or guanfacine. In another aspect, the first agent is selected from metyrapone; ketoconazole; mitotane; aminoglutethimide; etomidate; mifepristone; cytadren; and fluconazole; and the second agent is clonidine or guanfacine. In another aspect, the first agent is selected from mifepristone; cytadren; and fluconazole; and the second agent is clonidine or guanfacine.

In another aspect, the first agent is mifepristone; and the second agent is clonidine. In another aspect, the first agent is mifepristone; and the second agent is guanfacine.

In another aspect, the first agent is cytadren; and the second agent is clonidine. In another aspect, the first agent is cytadren; and the second agent is guanfacine.

In another aspect, the first agent is fluconazole; and the second agent is clonidine. In another aspect, the first agent is fluconazole; and the second agent is guanfacine.

In another aspect, the first agent is a cortisol inhibitor and the second agent is an azapirone. In another aspect, the first agent is selected from metyrapone; ketoconazole; mitotane; aminoglutethimide; etomidate; mifepristone; cytadren; and fluconazole; and the second agent is an azapirone. In another aspect, the first agent is selected from mifepristone; cytadren; and fluconazole; and the second agent is an azapirone. In another aspect, the first agent is a cortisol inhibitor and the second agent is buspirone or tandospirone. In another aspect, the first agent is selected from metyrapone; ketoconazole; mitotane; aminoglutethimide; etomidate; mifepristone; cytadren; and fluconazole; and the second agent is buspirone or tandospirone. In another aspect, the first agent is selected from mifepristone; cytadren; and fluconazole; and the second agent is buspirone or tandospirone.

In some embodiments the pharmaceutical composition further comprises a third agent.

The pharmaceutical composition of the present invention may be formulated for administration by one or more of the oral, rectal, parenteral, topical, intradermal, subcutaneous, intramuscular, intravenous, intraosseous, intraperitoneal, intrathecal, intranasal, epidural, intracardiac, intraarticular, intracavernous, intravitreal, intravaginal, intracervical, pulmonary and inhalation routes. The methods described herein may include or exclude any of the listed routes. In yet other embodiments, the composition may be formulated as one or more of the following dosage forms: a liquid, solution, suspension, emulsion, elixir, syrup, drop, powder electuary, granule, capsule, tablet, lozenge, pastille, gel, paste, ointment, cream, lotion, oil, foam, spray, mist, or aerosols. The methods described herein may include or exclude any of the listed dosage forms.

Another aspect of the present invention provides methods of treating patients who are suffering from disorders associated with aberrant activity in the HPA axis, the method comprising: (a) identifying a patient in need of treatment; and (b) administering to the patient a therapeutically effective amount of a composition described herein. The methods of the present invention may also comprise the use of a therapeutically effective amount of any of the compositions described herein in the manufacture of a medicament for treating a patient who is suffering from a disorder associated with aberrant activity in the HPA axis. For example, the disorders may include, but are not limited to, addiction to a substance, addiction to an activity, substance use disorders, mood disorders, anxiety disorder, bipolar disorder, insomnia, posttraumatic stress syndrome, borderline personality disorder, ADHD, major depressive disorder, burnout, chronic fatigue syndrome, fibromyalgia, irritable bowel syndrome, obesity, depression, or schizophrenia. The terms “addiction” and related disorders, “substance abuse disorder”, and “substance use disorder” are commonly used interchangeably by persons of ordinary skill in the art and in relevant literature.

Another aspect of the present invention provides methods of treating patients who are suffering from disorders associated with aberrant activity in the HPA axis, the method comprising: (a) identifying a patient in need of treatment; and (b) administering to the patient a therapeutically effective amount of a composition described herein. The methods of the present invention may also comprise the use of a therapeutically effective amount of any of the compositions described herein in the manufacture of a medicament for treating a patient who is suffering from a disorder associated with aberrant activity in the HPA axis. For example, the disorders may include, but are not limited to, addiction to a substance (e.g., cocaine, amphetamines, methamphetamine, methylphenidate, heroin, codeine, hydrocodone, nicotine, alcohol, prescription medication (e.g., Percodan®, Percoset®), marijuana, tobacco, methadone, food), addiction to an activity (e.g., gambling, sex, eating), substance use disorders, mood disorders, anxiety disorders, bipolar disorder, sleep disorders, insomnia, posttraumatic stress syndrome, borderline personality disorder, disruptive behavior disorders, ADHD, major depressive disorder, burnout, chronic fatigue syndrome, fibromyalgia, irritable bowel syndrome, eating disorders (e.g., Prader Willi Syndrome), obesity, depression, menopause, premenstrual syndrome (PMS), obsessive compulsive disorder (OCD),social anxiety, generalized anxiety disorder, dysthymia, or schizophrenia.

### DETAILED DESCRIPTION

HPA axis: The hypothalamic-pituitary-adrenal axis (HPA axis) includes positive and negative feedback interactions among three endocrine glands: the hypothalamus, the pituitary gland, and the adrenal glands that form the neuroendocrine system. Hormones released by the endocrine glands control reactions to stress, regulation of body processes like digestion, the immune system, mood and emotions, sexuality and energy storage and expenditure.

Corticotropin Releasing Hormone (CRH or CRF) is secreted by the paraventricular nucleus (PVN) of the hypothalamus in response to stress. Other factors that influence release of CRH include physical activity, illness, blood levels of cortisol and circadian rhythm. Stress activates the HPA axis under influence of neurotransmitters like dopamine, serotonin and norepinephrine (noradrenaline). Chronic stress activates the HPA axis in different ways depending on a number of factors, including whether the stressor is controllable, a threat to physical integrity, trauma, individual's physiology, quality of social interactions etc. For example, oxytocin secreted under influence of positive social interactions suppresses the HPA axis and counteracts stress.

In healthy individuals, cortisol levels show peculiar diurnal changes wherein the levels peak soon after waking up, gradually fall during late morning and mid-day, rise again in late afternoon and fall again in late evening, reaching a trough during the middle of the night. Abnormality in this cycle leads to pathological conditions. For example, flattened cortisol cycle causes chronic fatigue syndrome, insomnia and burnout and increased production of cortisol mediates alarm reactions to stress, general adaptation syndrome, immune suppression, etc.

Cushing's syndrome, Cushing's disease, pseudo-Cushing's syndrome or pituitary or ectopic tumor are characterized by increased levels of cortisol in plasma. On the other side of spectrum are conditions like Sheehan's syndrome, pituitary tumor, Addison's disease, Nelson's syndrome featured by decreased levels of cortisol in plasma.

Stress response normally controls maintenance of homeostasis, in the presence of real or perceived challenges, via activation of a complex range of responses involving the endocrine, nervous, and immune systems. Therefore, inappropriate regulation of the stress response is linked to a wide array of pathologies including autoimmune disease, hypertension, affective disorders, and major depression. Abnormal function of the HPA axis may cause or contribute to an addiction to a substance, substance use disorder, addiction to an activity, mood disorder, anxiety disorders, bipolar disorder, insomnia, posttraumatic stress syndrome, borderline personality disorder, ADHD, major depressive disorder, burnout, chronic fatigue syndrome, fibromyalgia, inflammatory and autoimmune disease, irritable bowel syndrome, obesity, depression, or schizophrenia. The HPA axis is also related to certain skin diseases like skin tumors and skin homeostasis. In addition, it is increasingly established that some disorders in adulthood, that are associated with childhood trauma, including physical, emotional, and sexual abuse and neglect, act via the HPA axis.

Stress response normally controls maintenance of homeostasis, in the presence of real or perceived challenges, via activation of a complex range of responses involving the endocrine, nervous, and immune systems. Therefore, inappropriate regulation of the stress response is linked to a wide array of pathologies including autoimmune disease, hypertension, affective disorders, and major depression. Abnormal function of the HPA axis may cause or contribute to an addiction to a substance (e.g., cocaine, amphetamines, methamphetamine, methylphenidate, heroin, codeine, hydrocodone, nicotine, alcohol, prescription medication (e.g., Percodan®, Percoset®), marijuana, tobacco, methadone, food), addiction to an activity (e.g., gambling, sex, eating), substance use disorder, mood disorder, anxiety disorder, bipolar disorder, insomnia, posttraumatic stress syndrome, borderline personality disorder, disruptive behavior disorders, ADHD, major depressive disorder, burnout, chronic fatigue syndrome, fibromyalgia, inflammatory and autoimmune disease, irritable bowel syndrome, eating disorders (e.g., Prader Willi Syndrome), obesity, depression, menopause, premenstrual syndrome (PMS), obsessive compulsive disorder (OCD),social anxiety, generalized anxiety disorder, dysthymia, or schizophrenia. The HPA axis is also related to certain skin diseases like skin tumors and skin homeostasis. In addition, it is increasingly established that some disorders in adulthood, that are associated with childhood trauma, including physical, emotional, and sexual abuse and neglect, act via the HPA axis.

Addiction to a substance, also known as chemical addiction, substance dependence, or substance use disorder is addiction including, but not limited to, stimulants (e.g., cocaine, amphetamines, methamphetamines, methylphenidate, and related stimulants), opiates (e.g., heroin, codeine, hydrocodone, and related opioid drugs), nicotine, alcohol, prescription medications (e.g., medications prescribed for pain management such as oxycodone, hydrocodone and other non-opioid pain medicines), naturally-occurring plant-derived drugs (e.g. marijuana, tobacco, and the addictive agents therein) and synthetic drugs (e.g. synthetic phenethylamines, including synthetic cathinones or synthetic hallucinogens, commonly known as “bath salts”, synthetic cannabinoids, also known as synthetic marijuana sold under commercial names like Bliss, Raving Dragon, Blue Light, Cloud 9, Blue Silk, Purple Tranquility, Charge, Zoom 2, Cosmic Blast, Aura, Disco Concentrate Bath Salts, Red Dove, Ivory Snow, Vanilla Sky, Ocean Burst, White Horse, Pure Ivory, Ivory Coast, Purple Wave, Energy 1, Snow Leopard, MDPK, Stardust, Star Dust, Magic, Tranquility Bath Salts, Super Coke, White Dove, Amped, White Knight, Rave, White Rush, SnowBlind, Zeus 2, Crystal Bubbly, Ivory Wave, Eight Ballz, White Lightening, White Water Rapid, Hurricane Charlie, Avalanche, White Girl, Bizarro, Blue Magic, Voodoo Powder, Silverback Bath Salts.)

Addiction to an activity, also known as physical addiction, behavioral or behavioural addiction, soft addiction, process addiction or non-substance-related addiction is addiction to activities including, but not limited to, eating, food, exercise, gambling, sex, viewing of pornography, use of computers, use of the internet, playing video games, work, spiritual obsession, cutting (self-harm), travel or shopping.

The present invention provides pharmaceutical compositions related to novel pharmaceutical combinations that include a first agent and a second agent useful for treating a patient who is suffering from a disorder associated with aberrant activity in the HPA axis. The pharmaceutical compositions described herein include novel pharmaceutical combinations of a first agent and a second agent.

The agents of the present invention may be categorized in various ways, and the compositions of the invention may include two or more agents of the same or different types. For example, the agents can be categorized as chemical compounds (e.g., benzodiazepines, topiramate and imidazole derivatives), although in some embodiments the first agent or the second agent include protein or protein-based molecules, such as mutant ligands (e.g., a ligand that binds but does not activate or fully activate its cognate receptor) or antibodies; or as nucleic acids or nucleic acid-based entities, such as antisense oligonucleotides or RNA molecules that mediate RNAi may also be used.

Previous work has demonstrated that the HPA axis plays an important role in drug addiction (Goeders, Psychoneuroendocrinology 22:237, 1997; Goeders, J Pharmacol. Exp. Ther. 301:785-789, 2002; Goeders, Psychoneuroendocrinology 27: 13-33, 2002; Goeders, Eur. Neuropsychopharmacology; 3:435-441, 2003). Accordingly, the present invention features compositions that represent combined therapeutic agents and methods of treating patients with these agents.

The first agent: Chemical compounds useful as a first agent in the present invention include, but are not limited to, mitotane, aminoglutethimide, etomidate and certain the compounds described in the International Application Publication Numbers WO2005118557, WO2005118581, WO2007024945, WO2007117982, WO2008076336, WO2009135651, WO2009156462, WO2010130773, WO2010130794, WO2010130796, WO2011061168, WO2011064376, and WO2011088188; United States Patent Application Publication Numbers 2012/0071512, 2012/0277215, 2013/0296309, 2013/0287789; U.S. Pat. Nos. 7,612,088; 8,030,334; 8,153,674; 8,314,097; 8,383,827; 8,436,035; 8,455,522; 8,519,134; 8,519,142; 8,541,404; 8,575,160; 8,680,079; 8,609,862; 8,685,960; the contents of which are incorporated by herein reference.

In some aspects, the chemical compounds useful as the first agent include metyrapone, metyrapol and the compounds described in the International Application Publication Numbers WO2007056618; WO2011159871; the contents of which are incorporated by herein reference.

In some aspects, the first agent includes imidazole derivatives, described by a compound of Formula I:

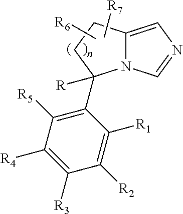

wherein

n is 1, or 2, or 3;

R is hydrogen, C₁-C₇ alkyl, or C₂-C₇ alkenyl, —COO—R₁₀, or —CONR₁₁R₁₂,

wherein the C₁-C₇ alkyl and C₂-C₇ alkenyl are optionally substituted by one to five substituents independently selected from the group consisting of —OR₈ and —NR₈R₉, wherein R₈ and R₉ are independently selected from the group consisting of hydrogen, C₁-C₇ alkyl, acyl, aryl and heteroaryl, each of which is further optionally substituted by one to four substituents independently selected from the group consisting of halo, C₁-C₇ alkoxy and C₁-C₇ alkyl; wherein R₁₀, R₁₁ and R ₁₂ are selected independently from the group consisting of hydrogen, C₁-C₇ alkyl, C₃-C₈ cycloalkyl, aryl, aryl-C₁-C₇ alkyl, C₁-C₇ haloalkyl and heteroaryl, each of which is further optionally substituted by one to four substituents independently selected from the group consisting of halo, hydroxyl, C₁-C₇ alkoxy, C₁-C₇ alkyl, and aryl, wherein R₁₁ and R ₁₂ taken together with the nitrogen atom to which they are attached optionally form a 3-8-membered ring;

R₁, R₂, R₃, R₄ and R₅ are selected independently from the group consisting of hydrogen, C₂-C₇ alkenyl, C₁-C₇ alkyl, C₃-C₈ cycloalkyl, halo, cyano, nitro, —NH₂, C₁-C₇ haloalkyl, C₁-C₇ alkoxy, C₃-C₈ cycloalkoxy, aryloxy, aryl, heteroaryl, —COOR₁₀, and —NR₁₃R₁₄, said C₁-C₇ alkyl, C₂-C₇ alkenyl, C₁-C₇ alkoxy, aryl and heteroaryl being further optionally substituted by one to three substituents selected from C₁-C₇ alkyl, hydroxyl, halo, C₁-C₇ alkoxy, nitro, cyano, C₁-C₇ dialkylamino, C₁-C₇ alkoxy-C₁-C₇ alkyl, and C₁-C₇ haloalkyl, said R₁₀ having the same meanings as defined above, said R₁₃ and R₁₄ are independently selected from the group consisting of hydrogen, C₁-C₇ alkyl, C₃-C₈ cycloalkyl, C₁-C₇ haloalkyl, C₁-C₇ haloalkoxy, aryl and cyano, with the proviso that no more than three of R₁, R₂, R₃, R₄ and R₅ are simultaneously hydrogen;

R₁₃ and R₁₄ taken together with the nitrogen atom to which they are attached optionally form a 3-8-membered ring;

R and R₁ taken together optionally form a 5-6-membered ring containing 0 or 1 heteroatoms selected from O, N, or S;

R₆ and R₇ are independently hydrogen, hydroxyl, C₁-C₇ alkyl, C₁-C₇ alkoxy, phenyl, or benzyl, wherein phenyl and benzyl are optionally substituted by one to four substituents independently selected from the group consisting of halo, C₁-C₇ alkoxy and C₁-C₇ alkyl;

when R₆ and R₇ are attached to the same carbon atom, they optionally form a moiety A represented by the following structure:

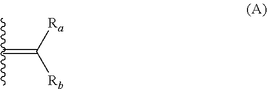

wherein Rₐ and R_{b} are independently hydrogen, C₁-C₇ alkyl, C₁-C₇ alkoxy, acyl, —COOR₁₅ or —COR₁₅, said R₁₅ being hydrogen, C₁-C₇ alkyl, C₁-C₇ haloalkyl, aryl, or —NH₂; or

when R₆ and R₇ are attached to the same carbon atom, they taken together with said carbon atom optionally form a 3-8-membered ring; or a pharmaceutically acceptable salt thereof: or an optical isomer thereof; or a mixture of optical isomers.

In other embodiments, the first agent includes the compound of formula II is

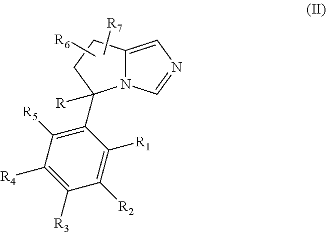

wherein

R is selected from the group consisting of: hydrogen, C₁-C₇ alkyl, and C₂-C₇ alkenyl,

R₁ is selected from F, Cl, Br and I,

R₂, R₃, R₄, and R ₅ are selected independently from the group consisting of hydrogen, C₂-C₇, alkenyl, C₁-C₇ alkyl, C₃-C₈ cycloalkyl, F, Cl, Br, I, cyano, nitro, H₂N—, C₁-C₇ haloalkyl, and C₁-C₇ alkoxy,

R₆, and R₇ are hydrogen,

or a pharmaceutically acceptable salt thereof, or a pharmaceutically acceptable salt, solvate, hydrate, polymorph, precursor, metabolite, or prodrug thereof.

The compositions and methods described herein may include or exclude any of the listed substitutions.

Yet other embodiments provides a first agent that includes (R)-4-(6,7-dihydro-5H-pyrrolo[1,2-c]imidazol-5-yl)-3-fluorobenzonitrile, the compound of Formula III

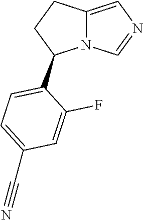 [SMILES: N#Cc1ccc([C@H]2CCc3cncn32)c(F)c1]

or analog, enantiomer thereof; or a pharmaceutically acceptable salt, solvate, hydrate, polymorph, precursor, metabolite, or prodrug thereof.

In some embodiments, the compound of formula I, formula II or formula III is administered as the first agent at doses ranging from about 0.01 mg to about 10 gm/day. An exemplary dose may be about 0.01 mg, about 0.02 mg, about 0.025 mg, about 0.05 mg, about 0.075 mg, about 0.1 mg, about 0.2 mg, about 0.25 mg, about 0.5 mg, about 0.75 mg, about 1 mg, about 2 mg, about 2.5 mg, about 5 mg, about 7.5 mg, about 10 mg, about 20 mg, about 25 mg, about 50 mg, about 75 mg, about 100 mg, about 200 mg, about 250 mg, about 500 mg, about 750 mg, about 1 g, about 2 g, about 2.5 g, about 5 g, about 7.5 g, or about 10 g per day, or any other value within this overall range.

In some embodiments, the chemical compounds useful as the first agent include, but are not limited to, steroidogenesis inhibitors (e.g. LCI699, compound of formula I, compound of formula II, compound of formula III, ketoconazole, 2S,4R enantiomer of ketoconazole, metyrapone, metyrapol, mitotane, aminoglutethimide, etomidate or a pharmaceutically acceptable salt, solvate, hydrate, polymorph, precursor, metabolite, prodrug or a derivative thereof). These agents are described by Morgan and Laufgraben, Expert Rev Endocrinol Metab. 8(2):183-193, 2013, which is incorporated herein by reference. The steroidogenesis inhibitors may decrease cortisol production in the adrenal gland through inhibition of one or more enzymes involved in steroid synthesis, or by other mechanisms of action. The steroidogenesis inhibitors exhibit a dose-dependent inhibition of cortisol production. In some embodiments, the compositions of the present invention completely inhibit cortisol production. In other embodiments, the compositions of the present invention partially inhibit cortisol production or reduce cortisol levels in the serum. In yet other embodiments, the compositions of present invention do not alter cortisol levels in the serum.

Mitotane inhibits several cholesterol side-chain cleavage enzymes like 11β-hydroxylase, 18-hydroxylase, 3-α hydroxylase, hydroxysteroid dehydrogenase and thereby reduces cortisol synthesis. It is also an adrenolytic agent at doses greater than 4 g per day, and is used most often for the treatment of adrenocortical carcinoma. In some embodiments, mitotane is administered as the first agent at doses ranging from about 0.1 mg to about 20 gm/day. Exemplary dose may be about 0.1 mg, about 0.2 mg, about 0.25 mg, about 0.5 mg, about 0.75 mg, about 1 mg, about 2 mg, about 2.5 mg, about 5 mg, about 7.5 mg, about 10 mg, about 20 mg, about 25 mg, about 50 mg, about 75 mg, about 100 mg, about 200 mg, about 250 mg, about 500 mg, about 750 mg, about 1 g, about 2 g, about 2.5 g, about 5 g, about 7.5 g, about 10 g or about 20 g per day or any other value within this overall range.

Aminoglutethimide was first introduced in 1959 as an anticonvulsant. Subsequently, it was discovered that it blocks conversion of cholesterol to pregnenolone, the first step in steroid hormone biosynthesis, by inhibiting the enzyme P450scc and consequently decreases synthesis of all hormonally active steroids. Furthermore, it inhibits aromatase, and thereby blocks generation of estrogens from androstenedione and testosterone. Because of this mechanism, it also inhibits estrogen and aldosterone production, and has been investigated in the treatment of breast cancer.

Ketoconazole is a widely used antifungal agent that inhibits various enzymes in adrenal cortisol synthesis, is effective in treating hypercortisolemia, but its use is limited by toxicities. Ketoconazole is a racemic compound of two cis-enantiomers: (2R,4S)-(+)-ketoconazole and (2S,4R)-(−)-ketoconazole. Recently, it has been found that the (2S,4R)-(−)-ketoconazole enantiomer has selective effect but minimal metabolic toxicity. In some embodiments, the first agent is ketoconazole, 2S,4R enantiomer of ketoconazole, or a pharmaceutically acceptable salt, solvate, hydrate, polymorph, precursor, metabolite, prodrug or a derivative thereof.

In some embodiments of the present invention, the agents that may be used as a first agent include, but are not limited to, LCI699, ketoconazole. 2S,4R enantiomer of ketoconazole, 2R,4S enantiomer of ketoconazole, metyrapone, metyrapol, mitotane, aminoglutethimide, etomidate, pasireotide, mifepristone and cabergoline or a pharmaceutically acceptable salt, solvate, hydrate, polymorph, precursor, metabolite, prodrug or a derivative thereof.

In some embodiments, aminoglutethimide is administered as the first agent at doses ranging from about 0.1 mg to about 20 gm/day. Exemplary dose may be about 0.1 mg, about 0.2 mg, about 0.25 mg, about 0.5 mg, about 0.75 mg, about 1 mg, about 2 mg, about 2.5 mg, about 5 mg, about 7.5 mg, about 10 mg, about 20 mg, about 25 mg, about 50 mg, about 75 mg, about 100 mg, about 200 mg, about 250 mg, about 500 mg, about 750 mg, about 1 g, about 2 g, about 2.5 g, about 5 g, about 7.5 g, about 10 g or about 20 g per day or any other value within this overall range. In some embodiments, aminoglutethimide is formulated as an injection. In an embodiment, a second agent is co-formulated with aminoglutethimide. In other embodiments, the second agent is co-administered separately using same of different route.

Etomidate is an intravenous medication used for anesthesia induction, inhibits cholesterol side-chain cleavage and 11-B hydroxylase and adrenal steroid synthesis. Studies in healthy subjects revealed that the infusion of etomidate resulted in significant suppression of cortisol levels after 5 h with maximal effects at 11 h. In some embodiments, etomidate is infused at a rate with the range of 0.001 mg/kg/h to 0.1 mg/kg/h. Exemplary rates of infusion may be about 0.001 mg/kg/h, about 0.002 mg/kg/h, about 0.0025 mg/kg/h, about 0.005 mg/kg/h, about 0.0075 mg/kg/h, about 0.01 mg/kg/h, about 0.02 mg/kg/h, about 0.025 mg/kg/h, about 0.05 mg/kg/h, about 0.075 mg/kg/h, about 0.01 mg/kg/h, or any other value within this overall range. In some embodiments, etomidate is formulated as an injectable composition. In another embodiment, a second agent is co-formulated with etomidate. In other embodiments, the second agent is co-administered separately using same or different route of administration. In some embodiments, the first agent and the second agent, described herein, are co-administered separately using the same or different route, using the same or different dosage form. In some embodiments, the first agent and the second agent, described herein, is combined in form of a unit dosage form. In some embodiments, the unit dose form includes a third agent in addition to the first agent and/or the second agent.

In some embodiments of the present invention, the agents that may be used as a first agent include, but are not limited to, a CRH/CRF-1 antagonist; an ACTH antagonist; or a cortisol synthesis inhibitor. Exemplary CRH/CRF-1 antagonists are selected from, but not limited to, antalarmin; pexacerfont; verucerfont; LWH-234; CP-154,536; NBI-27914; and R-121,919. Exemplary ACTH antagonists are selected from, but not limited to, bromocriptine; cabergoline; somastatin analogs (e.g., octreotide, pasireotide); retinoic acid; and cyproheptadine. Exemplary cortisol synthesis inhibitors or cortisol receptor antagonists are selected from, but not limited to, metyrapone; metyrapol; ketoconazole; mitotane; aminoglutethimide; etomidate; mifepristone; cytadren; and fluconazole.

The second agent: Chemical compounds useful as second agents include, but are not limited to, sedatives, hypnotics, anxiolytics and anticonvulsants.

In some embodiments, the second agent is selected from the group consisting of barbiturates, benzodiazepines, nonbenzodiazepine sedatives, orexin antagonists, antidepressants, antihistamines, herbal sedatives, methaqualone and analogues, other sedatives, antipsychotics, serotonin antagonists and reuptake inhibitors. The barbiturates that are suitable as the second agent include, but are not limited to, benzylbutylbarbiturate (designer drug), amobarbital, pentobarbital, secobarbital and phenobarbital. The benzodiazepines that are suitable as the second agent include but are not limited to clonazepam, diazepam, estazolam, flunitrazepam, lorazepam, midazolam, nitrazepam, oxazepam, triazolam, temazepam, chlordiazepoxide and alprazolam. The nonbenzodiazepine sedatives that are suitable as the second agent include, but are not limited to, eszopiclone, zaleplon, zolpidem and zopiclone. The orexin antagonists that are suitable as the second agent include, but are not limited to, suvorexant. The antihistamines that are suitable as the second agent include, but are not limited to, diphenhydramine, dimenhydrinate, doxylamine, mirtazapine and promethazine. The herbal sedatives that are suitable as the second agent include but are not limited to Duboisia hopwoodii, Chamomile, Prostanthera striatiflora, catnip, kava (Piper methysticum), valerian, cannabis, passiflora spp. (passiflora incamata), and validol. The methaqualone and analogues that are suitable as the second agent include, but are not limited to, afloqualone, cloroqualone, diproqualone, etaqualone, methaqualone, methaqualone, methylmethaqualone, mebroqualone, mecloqualone and nitromethaqualone. Other sedatives that are suitable as the second agent include, but are not limited to, 2-methyl-2-butanol (2M2B), chloral hydrate, etizolam (benzodiazepine analog), alcohol, trazodone, opiates and opioids, glutethimide and GHB. The serotonin antagonists and reuptake inhibitors that are suitable as the second agent include, but are not limited to, trazodone. The tricyclic antidepressants that are suitable as the second agent include, but are not limited to, amitriptyline, doxepin and trimipramine. The tetracyclic antidepressants that are suitable as the second agent include, but are not limited to, mianserin and mirtazapine. The antipsychotics that are suitable as the second agent include, but are not limited to, adinazolam, alprazolam, clonazepam, chlordiazepoxide, climazolam, clorazepate, diazepam, estazolam, flunitrazepam, flurazepam, halazepam, loprazolam, lorazepam, lormetazepam, midazolam, nimetazepam, nitrazepam, oxazepam, prazepam, temazepam, triazolam or a pharmaceutically acceptable salt thereof the methods described herein may include or exclude any of the listed agents.

In some embodiments of the present invention, the second agent may comprise one or more agents that target the prefrontal cortex by targeting GABA. Benzodiazepines (e.g., oxazepam) are one class of drugs useful in that regard. (Baldessarini, In: Hardman et al. (Eds), Goodman & Gilman's The Pharmacological Basis of Therapeutics, McGraw-Hill, New York, pp. 399-430, 1996). As some of the major symptoms associated with cocaine withdrawal often include severe anxiety, restlessness and agitation (Crowley, In: Fisher et al. (Eds), Cocaine: Clinical and Biobehavioral Aspects, Oxford University Press, New York, pp. 193-211, 1987; Gawin and Ellinwood, Ann. Rev. Med. 40:149-161, 1989; Tarr and Macklin, Pediatric Clinics of North America 34:319-331, 1987), benzodiazepines may be useful for alleviating these negative symptoms during the early stages of withdrawal. These drugs are also useful in the emergency room for the treatment of some of the medical complications associated with cocaine intoxication, since convulsions are often apparent following an acute overdose. These seizures can sometimes be effectively treated with intravenous diazepam (Valium®) (Gay, J Psychoactive Drugs .L1.:297-318, 1981; Tarr and Macklin, Pediatric Clinics of North America 34:319-331, 1987), and diazepam can be used in the combination therapies described herein. Benzodiazepine receptor expression can be assessed using methods known in the art. For example, receptors can be labeled with [³H]PK11195 (see Javaid et al., Biol. Psychiatry 36:44-50, 1994; see also Chesley et al., J Clin. Psychiatry 21:404-406, 1990). The data described below further suggests that benzodiazepines mediate certain aspects of cocaine reinforcement in rats.

Useful benzodiazepines or agents that target the prefrontal cortex include, but are not limited to, oxazepam, as chlordiazepoxide, mirtazapine, atomoxetine, gabapentin, muscimol, progabide, riluzole, baclofen, vigabatrin, valproic acid, tiagabine, lamotrigine, phenytoin, carbamazepine, and topiramate.

Where an agent that inhibits activity in the sympathetic nervous system is included, that agent can be sotalol, imolol, carteolol, carvedilol, nadolol, nadol/bendroflunetazide, propranolol, propranolol/HCTZ, betaxolol, penbutolol, metoprolol, labetalol, acebutolol, atenolol/HCTZ, atenolol, timolol/HCTZ, metoprolol, labetalol, pindolol, bisoprolol, bisoprolol/HCTZ, esmolol, or combinations thereof.

In another aspect, the second agent is an antidepressant; benzodiazepine; beta-blocker; antipsychotic; alpha-adrenergic agonist; 5-HT 1 A agonist; azapirone; mebicarum; fabomitizole; selank; bromantane; emoxypine; hydroxyzine; pregbalin; methyl isovalerate; cannabidiol; tetrahydrocannabinol; propofol; BNC210; CL-218,872; L-838,417; SL-651,498; 532212; or PH94B.

In another aspect, the antidepressant is selected from serotonin selective reuptake inhibitors (SSRIs); serotonin norepinephrine reuptake inhibitors (SNRIs); serotonin modulators and stimulators (SMSs); serotonin antagonists and reuptake inhibitors (SARIs); norepinephrine reuptake inhibitors (NRIs); tricyclic antidepressants (TCAs); tetracyclic antidepressants (TeCAs); monoamine oxidase inhibitors (MAOIs); norepinephrine dopamine reuptake inhibitors; agomelatine; bifemelane; tandospirone; and teniloxazine. In another aspect, the antidepressant is selected from serotonin selective reuptake inhibitors (SSRIs); serotonin norepinephrine reuptake inhibitors (SNRIs); serotonin modulators and stimulators (SMSs); agomelatine; bifemelane; tandospirone; and teniloxazine.

Exemplary SSRIs are selected from, but not limited to, citalopram; escitalopram; paroxetine; fluoxetine; fluvoxamine; and sertraline. Exemplary SNRIs are selected from, but not limited to, desvenlafaxine; duloxetine; levomilnacipran; milnacipran; tofenacin; and venlafaxine. Exemplary SMSs are selected from, but not limited to, vilazodone and vortioxetine. Exemplary SARIs are selected from, but not limited to, etoperidone; nefazodone; and trazodone. Exemplary NRIs are selected from, but not limited to, reboxetine; viloxazine; and atromoxetine. Exemplary TCAs are selected from, but not limited to, amitriptyline; amitriptylinoxide; clomipramine; desipramine; dibenzepin; dimetacrine; dosulepin; doxepin; imipramine; lofepramine; melitracen; nitroxazepine; nortriptyline; noxiptiline; pipofezine; proptriptyline; trimipramine; opipramol; and tianeptine. Exemplary TeCAs are selected from, but not limited to, amoxapine; maprotiline; mianserin; mirtazapine; and setiptiline. Exemplary MAOIs are selected from, but not limited to, isocarboxazid; phenelzine; tranylcypromine; selegiline; metralindole; moclobemide; pirlindole; and toloxatone.

Exemplary benzodiazepines are selected from, but not limited to, oxazepam; chlordiazepoxide; mirtazapine; atomoxetine; gabapentin; (Neurontin™); muscimol; progabide; riluzole; baclofen; vigabatrin; valproic acid (Depakote™); tiagabine (Gabitril™); lamotrigine (Lamictal™); phenytoin (Dilantin™); carbamazepine (Tegretol™); topiramate (Topamax™); lorazepam; (Ativan®), prazepam (Centrax®); flurazepam (Dalmane®); clonazepam (Klonopin®); chlordiazepoxide (Librium®); halazepam (Paxipam®); temezepam (Restoril®); clorazapate; (Tranxene®), diazepam (Valium®), and alprazolam (Xanax®).

Exemplary beta-blockers are selected from, but not limited to, propanolol; bucindolol; carteolol; carvedilol; labetol; nadolol; oxprenolol; penbutolol; pindolol; sotalol; timolol; acebutolol; atenolol; betaxolol; bisoprolol; celiprolol; esmolol; metoprolol; nebivolol; butaxamine; ICI-118,551; and SR-59230A.

Exemplary antipsychotics are selected from, but not limited to, benperidol; bromperidol; droperidol; haloperidol; timiperone; diphenylbutylpiperidine; fluspirilene; penfluridol; pimozide; phenothiazines; chlorpromazine; cyamemazine; dixyrazine; fluphenazine; levomepromazine; perazine; pericyazine; perphenazine; pipotiazine; prochlorperazine; promethazine; prothipendyl; thioproperazine; trifluoperazine; chlorprothixene; clopenthixol; flupentixol; thiothixene; zuclopenthixol; clotiapine; loxapine; prothipendyl; carpipramine; clocapramine; molindone; mosapramine; sulpiride; sultopride; veralipride; amisulpride; amoxapine; arpiprazole; asenapine; cariprazine; clozapine; blonaserin; iloperidone; lurasidone; melperone; nemonapride; olanzapine; paliperidone; perospirone; quetiapine; remoxapride; risperidone; sertindole; trimipramine; ziprasidone; zotepine; brexpiprazole; ITI-007; pimavanserin; and RP5063.

Exemplary alpha-adrenergic agonists are selected from, but not limited to, clonidine and guanfacine.

Exemplary azapirones are selected from, but not limited to, buspirone and tandospirone.

Nucleic acid-based therapeutics: The therapeutic agents useful in treating the conditions described herein can also be nucleic acids. These nucleic acids can serve as the first agent that targets the HPA axis by inhibiting, directly or indirectly, the expression of CRH, ACTH, or cortisol, and they can serve as the second agent that targets the prefrontal cortex by increasing GABA.

The nucleic acids useful in the present invention may be “isolated” or “purified” (i.e., no longer associated with some or all of the flanking nucleic acid sequences or cellular components with which the nucleic acid is naturally associated in vivo). For example, with respect to a cell, tissue, or organism with which it was once naturally associated, a nucleic acid sequence useful as a therapeutic agent can be at least 50% pure (e.g., 60%, 70%, 75%, 80%, 85%, 90%, 95%, 98%, or 99% pure). Where a naturally occurring or modified nucleic acid sequence (e.g., a cDNA) is administered, it may include some of the 5′ or 3′ non-coding sequence associated with the naturally occurring gene. For example, an isolated nucleic acid (DNA or RNA) can include some or all of the 5′ or 3′ non-coding sequence that flanks the coding sequence (e.g., the DNA sequence that is transcribed into, or the RNA sequence that gives rise to, the promoter or an enhancer in the mRNA). For example, an isolated nucleic acid can contain less than about 5 kb (e.g., less than about 4 kb, 3 kb, 2 kb, 1 kb, 0.5 kb, or 0.1 kb) of the 5′ and/or 3′ sequence that naturally flanks the nucleic acid molecule in a cell in which the nucleic acid naturally occurs. In the event the nucleic acid is RNA or mRNA, it is “isolated” or “purified” from a natural source (e.g., a tissue) or a cell culture when it is substantially free of the cellular components with which it naturally associates in the cell and, if the cell was cultured, the cellular components and medium in which the cell was cultured (e.g., when the RNA or mRNA is in a form that contains less than about 20%, 10%, 5%, 1%, or less, of other cellular components or culture medium). When chemically synthesized, a nucleic acid (DNA or RNA) is “isolated” or “purified” when it is substantially free of the chemical precursors or other chemicals used in its synthesis (e.g., when the nucleic acid is in a form that contains less than about 20%, 10%, 5%, 1%, or less, of chemical precursors or other chemicals).

Nucleic acids useful in the compositions and methods described herein can be double-stranded or single-stranded and can, therefore, either be a sense strand, an antisense strand, or a portion (i.e., a fragment) of either the sense or the antisense strand. The nucleic acids can be synthesized using standard nucleotides or nucleotide analogs or derivatives (e.g., inosine, phosphorothioate, or acridine substituted nucleotides), which can alter the nucleic acid's ability to pair with complementary sequences or to resist nucleases. The stability or solubility of a nucleic acid can be altered (e.g., improved) by modifying the nucleic acid's base moiety, sugar moiety, or phosphate backbone. For example, the nucleic acids of the invention can be modified as taught by Toulme (Nature Biotech. 19: 17, 2001) or Faria et al. (Nature Biotech. 19:40-44, 2001), and the deoxyribose phosphate backbone of nucleic acids can be modified to generate peptide nucleic acids (PNAs; see Hyrup et al., Bioorganic & Medicinal Chemistry 4:5-23, 1996).

PNAs are nucleic acid “mimics;” the molecule's natural backbone is replaced by a pseudopeptide backbone and only the four nucleotide bases are retained. This allows specific hybridization to DNA and RNA under conditions of low ionic strength. PNAs can be synthesized using standard solid phase peptide synthesis protocols as described, for example by Hyrup et al. (supra) and Perry-O'Keefe et al. (Proc. Natl. Acad. Sci. USA 93:14670-675). PNAs of the nucleic acids described herein can be used in therapeutic and diagnostic applications. For example, PNAs can be used as antisense or antigene agents for sequence-specific modulation of gene expression by, for example, inducing transcription or translation arrest or inhibiting replication.

The nucleic acids can be incorporated into a vector (e.g., an autonomously replicating plasmid or virus) prior to administration to a patient, and such vectors are within the scope of the present invention. The invention also encompasses genetic constructs (e.g., plasmids, cosmids, and other vectors that transport nucleic acids) that include a nucleic acid of the invention in a sense or antisense orientation. The nucleic acids can be operably linked to a regulatory sequence (e.g., a promoter, enhancer, or other expression control sequence, such as a polyadenylation signal) that facilitates expression of the nucleic acid. The vector can replicate autonomously or integrate into a host genome, and can be a viral vector, such as a replication defective retrovirus, an adenovirus, or an adeno-associated virus. In addition, when present, the regulatory sequence can direct constitutive or tissue-specific expression of the nucleic acid.

The nucleic acids can be antisense oligonucleotides. While “antisense” to the coding strand of the targeted sequence, they need not bind to a coding sequence; they can also bind to a noncoding region (e.g., the 5′ or 3′ untranslated region). For example, the antisense oligonucleotide can be complementary to the region surrounding the translation start site of an mRNA (e.g., between the −10 and +10 regions of a target gene of interest or in or around the polyadenylation signal). Moreover, gene expression can be inhibited by targeting nucleotide sequences complementary to regulatory regions (e.g., promoters and/or enhancers) to form triple helical structures that prevent transcription of the gene in target cells (see generally, Helene, Anticancer Drug Des. 6:569-84, 1991; Helene, Ann. N Y Acad. Sci. 660:27-36, 1992; and Maher, Bioassays 14:807-15, 1992). The sequences that can be targeted successfully in this manner can be increased by creating a so-called “switchback” nucleic acid. Switchback molecules are synthesized in an alternating 5′-3′, 3′-5′ manner, such that they base pair with first one strand of a duplex and then the other, eliminating the necessity for a sizable stretch of either purines or pyrimidines on one strand of a duplex.

Fragments having as few as 9-10 nucleotides (e.g., 12-14, 15-17, 18-20, 21-23, or 24-27 nucleotides; siRNAs typically have 21 nucleotides) can be useful and are within the scope of the invention.

In other embodiments, antisense nucleic acids can be anomeric nucleic acids, which form specific double-stranded hybrids with complementary RNA in which, contrary to the usual b-units, the strands run parallel to each other (Gaultier et al., Nucleic Acids Res. 15:6625-6641, 1987; see also Tanaka et al., Nucl. Acids Res. 22:3069-3074, 1994). Alternatively, antisense nucleic acids can comprise a 2′-o-methylribonucleotide (Inoue et al., Nucleic Acids Res. 15:6131-6148, 1987) or a chimeric RNA-DNA analogue (Inoue et al., FEES Lett. 215:327-330, 1987).

Antibodies: Antibodies and antigen binding fragments thereof useful as therapeutic agents in the present compositions. These antibodies may be of the G class (IgG), but IgM, IgD, IgA, and IgE antibodies can also be used; what is required is that the antibodies specifically bind a target described herein and alter that target—whether by enhancing or inhibiting its activity—in a way that, in accordance with our findings, confers a clinical benefit on a patient to whom they are administered. The antibodies can be polyclonal or monoclonal antibodies, and we use the terms “antibody” and “antibodies” to refer to whole antibodies or fragments thereof that are, or that include, an antigen-binding domain of the whole antibody. For example, useful antibodies can lack the Fe portion; can be single chain antibodies; or can be fragments consisting of (or consisting essentially of) the variable, antigen-binding domain of the antibody. The antibodies can by humanized (by, for example, CDR grafting) or fully human.

Methods of producing antibodies are well known in the art. For example, as noted above, human monoclonal antibodies can be generated in transgenic mice carrying the human immunoglobulin genes rather than those of the mouse. Splenocytes obtained from these mice (after immunization with an antigen of interest) can be used to produce hybridomas that secrete human mAbs with specific affinities for epitopes from a human protein (see, e.g., WO 91/00906, WO 91/10741; WO 92/03918; WO 92/03917; Lonberg et al., Nature 368:856-859, 1994; Green et al., Nature Genet. 7: 13-21, 1994; Morrison et al. Proc. Natl. Acad. Sci. USA 81:6851-6855, 1994; Bruggeman et al., Immunol. 7:33-40, 1993; Tuaillon et al., Proc. Natl. Acad. Sci. USA 90:3720-3724, 1993; and Bruggeman et al., Eur. J. Immunol 21:1323-1326, 1991).

The antibody can also be one in which the variable region, or a portion thereof (e.g., a CDR), is generated in a non-human organism (e.g., a rat or mouse). Thus, the invention encompasses chimeric, CDR-grafted, and humanized antibodies and antibodies that are generated in a non-human organism and then modified (in, e.g., the variable framework or constant region) to decrease antigenicity in a human. Chimeric antibodies (i.e., antibodies in which different portions are derived from different animal species (e.g., the variable region of a murine mAb and the constant region of a human immunoglobulin) can be produced by recombinant techniques known in the art. For example, a gene encoding the Fe constant region of a murine (or other species) monoclonal antibody molecule can be digested with restriction enzymes to remove the region encoding the murine Fe, and the equivalent portion of a gene encoding a human Fe constant region can be substituted therefor (see European Patent Application Nos. 125,023; 184,187; 171,496; and 173,494; see also WO 86/01533; U.S. Pat. No. 4,816,567; Better et al., Science 240:1041-1043, 1988; Liu et al., Proc. Natl. Acad. Sci. USA 84:3439-3443, 1987; Liu et al., J Immunol. 139:3521-3526, 1987; Sun et al., Proc. Natl. Acad. Sci. USA 84:214-218, 1987; Nishimura et al., Cancer Res. 47:999-1005, 1987; Wood et al., Nature 314:446-449, 1985; Shaw et al., J Natl. Cancer Inst. 80:1553-1559, 1988; Morrison et al., Proc. Natl. Acad. Sci. USA 81:6851, 1984; Neuberger et al., Nature 312:604, 1984; and Takeda et al., Nature 314:452, 1984).

An antigen-binding fragment of the invention can be: (i) a Fab fragment (i.e., a monovalent fragment consisting of the VL, VH, CL and CH1 domains); (ii) a F(ab′)₂ fragment (i.e., a bivalent fragment containing two Fab fragments linked by a disulfide bond at the hinge region); (iii) a Fd fragment consisting of the VH and CH1 domains; (iv) a Fv fragment consisting of the VL and VH domains of a single arm of an antibody, (v) a dAb fragment (Ward et al., Nature 341:544-546, 1989), which consists of a VH domain; and (vi) an isolated complementarity determining region (CDR).

Expression vectors can be used to produce the proteins of the invention, including antibodies, ex vivo (e.g., the proteins of the invention can be purified from expression systems such as those described herein) or in vivo (in, for example, whole organisms).

Formulations and Dosages:

In some embodiments, the compositions of the present invention do not alter cortisol levels in the serum. In other embodiments, low doses of the first agent and/or the second agent used in the composition alleviates side effects known to be associated with use of high doses of the agents. In some embodiments, the first agent and the second agent, described herein, are separately co-administered by same or different route. In some embodiments, the first agent and the second agent, described herein, are combined in form of a unit dosage form. In some embodiments, the unit dose form includes a third agent in addition to the first agent and/or the second agent.

Pharmaceutical compositions comprising the first agent and the second agent can be administered to a patient at therapeutically effective doses to prevent, treat or ameliorate addiction to a substance, an addiction to an activity, substance use disorders, other psychiatric disorders like mood disorders, anxiety disorder, bipolar disorder, insomnia, posttraumatic stress syndrome, borderline personality disorder, ADHD, major depressive disorder, burnout, chronic fatigue syndrome, fibromyalgia, inflammatory and autoimmune disease, irritable bowel syndrome, obesity, depression, or schizophrenia, certain skin diseases like skin tumors and skin homeostasis, or the associated conditions in adulthood, that are associated with childhood trauma, including physical, emotional, and sexual abuse and neglect. A therapeutically effective dose refers to an amount of the agent or combination of agents sufficient to improve at least one of the signs or symptoms of the addiction to a substance or addiction to an activity, substance use disorder, the psychiatric disorder, the associated conditions. For example, therapeutic amount sufficient to treat addiction is the amount of either the first agent or the second agent, in amount sufficient to reduce symptoms of addiction by 10% to 100%.

Pharmaceutical compositions comprising the first agent and the second agent can be administered to a patient at therapeutically effective doses to prevent, treat or ameliorate addiction to a substance (e.g., cocaine, amphetamines, methamphetamine, methylphenidate, heroin, codeine, hydrocodone, nicotine, alcohol, prescription medication (e.g., Percodan®, Percoset®), marijuana, tobacco, methadone, food), addiction to an activity (e.g., gambling, sex, eating), substance use disorders, mood disorders, anxiety disorders, bipolar disorder, sleep disorders, insomnia, posttraumatic stress syndrome, borderline personality disorder, disruptive behavior disorders, ADHD, major depressive disorder, burnout, chronic fatigue syndrome, fibromyalgia, irritable bowel syndrome, eating disorders (e.g., Prader Willi Syndrome), obesity, depression, menopause, premenstrual syndrome (PMS), obsessive compulsive disorder (OCD),social anxiety, generalized anxiety disorder, dysthymia, or schizophrenia, certain skin diseases like skin tumors and skin homeostasis, or the associated conditions in adulthood, that are associated with childhood trauma, including physical, emotional, and sexual abuse and neglect. A therapeutically effective dose refers to an amount of the agent or combination of agents sufficient to improve at least one of the signs or symptoms of the addiction to a substance or addiction to an activity, substance use disorders, the psychiatric disorder, the associated conditions. For example, therapeutic amount sufficient to treat addiction is the amount of either the first agent or the second agent, in amount sufficient to reduce symptoms of addiction by 10% to 100%.

Pharmaceutical compositions comprising the first agent and the second agent for use in accordance with the present invention may be formulated in a conventional manner using one or more physiologically acceptable carriers or excipients. The excipients include, but are not limited to, pharmaceutical acceptable carriers, diluents, adjuvants, fillers, buffers, preservatives, lubricants, solubilizers, surfactants, wetting agents, masking agents, coloring agents, flavoring agents, and sweetening agents. Also, as described herein, such formulation may also include other active agents, for example, other therapeutic or prophylactic agents.

The formulations comprising the first agent and the second agent may be prepared by methods well-known in the art of pharmacy. The formulation may be prepared to provide for rapid release, immediate release, slow release, delayed release, timed release, sustained release, extended release; or a combination thereof. Formulations may be in the form of liquids, solutions, suspensions, emulsions, elixirs, syrups, drops, powders, electuaries, granules, capsules, tablets, lozenges, pastilles, gels, pastes, ointments, creams, lotions, oils, foams, sprays, mists, or aerosols. The first agent and the second agent may be formulated together as a single dosage unit or may be formulated separately as distinct dosage units as a similar or different dosage form.

Any suitable concentration of the first agent and the second agent may be used, where the active pharmaceutical ingredient is administered in an effective amount to achieve its intended purpose. Determination of a therapeutically effective amount for a particular active ingredient is well within the capability of persons skilled in the art. In general, the dose may comprise about 0.005 mg to about 5 g/kg/day of the first agent and about 0.005 mg to about 5 g/kg/day of the second agent.

Many of the agents useful in the context of the present invention have been used previously to treat patients for other reasons. Where dosing information is available, it can be used to determine effective doses of the agents in the presently-described combinations. In some embodiments, the dose used to treat a patient for an addiction, one of the other psychiatric disorders described herein, and/or a related condition, may be the same as the dose that has been used previously for another indication. In some embodiments, the dose used to treat a patient for an addiction, one of the other psychiatric disorders described herein, and/or a related condition, may be more than the dose that has been used previously for another indication. In some embodiments, the dose used to treat a patient for an addiction, one of the other psychiatric disorders described herein, and/or a related condition, may be less than the dose that has been used previously for another indication. The effective doses may also differ. For example, the effective dosages required in connection with the combination therapies described herein may be less than those previously proven safe and effective.

Toxicity and therapeutic efficacy of the agents described herein can be determined, as necessary, by standard pharmaceutical procedures in cell cultures or experimental animals. For example, laboratory animals such as rodents and non-human primates can be used to determine the LD₅₀ (the dose lethal to 50% of the population) and the ED₅₀ (the dose therapeutically effective in 50% of the population). The dose ratio between toxic and therapeutic effects is the therapeutic index, which can be expressed as the ratio LD₅₀:ED₅₀. Compounds that exhibit large therapeutic indices are typically preferred.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range, depending upon the dosage form employed and the route of administration utilized. In some embodiments, any compound used in the method of the present invention, the therapeutically effective dose may be estimated initially from cell culture assays (e.g., assays designed to determine whether a nucleic acid, nucleic acid-based agent, or a protein such as an antibody inhibits (or stimulates) the expression or activity of the ligand or receptor it is intended to inhibit (or stimulate)). In some embodiments, for any compound used in the method of the present invention, the therapeutically effective dose may be estimated based on experimental data in laboratory animals, including but not limited to rodents, rabbits, pigs, dogs and non-human primates. In some embodiments, for any compound used in the method of the present invention, the therapeutically effective dose may be estimated based on one or more human clinical trials.

A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses (e.g., therapeutically effective doses) in humans. Levels in plasma can be measured, for example, by high performance liquid chromatography (“HPLC”).

One of the greatest concerns in the treatment of drug addiction is the high rate of recidivism. This phenomenon can be tested in animals during reinstatement, which is a widely regarded preclinical model of the propensity to relapse to substance abuse, and animal models of reinstatement can be used to further determine and define effective doses of the agents described herein. For example, animals can be taught to self-administer a drug until stable drug intake is maintained and then subjected to prolonged periods of extinction training or abstinence. Once the criteria for extinction are met, or following a specified period of abstinence, the ability of specific stimuli to reinstate responding on the manipulandum previously associated with the delivery of drug infusions is taken as a measure of drug seeking. This reinstatement of drug-seeking behavior can be elicited by priming injections of the drug itself in rats and monkeys (Stewart, J Psychiatr. Neurosci. 25: 125-136, 2000) or by exposure to brief periods of intermittent electric footshock in rats (Shaham et al., Brain Res. Rev. 33:13-33, 2000; Stewart, J Psychiatr. Neurosci. 25:125-136, 2000). Acute re-exposure to the self-administered drug (de Wit, Exp. Clin. Psychopharmacol. 4:5-10, 1996) and exposure to stress (Shiffman and Wills, Coping and Substance Abuse, Academic Press, Orlando, 1985; Lamon and Alonzo, Addict. Behav. 22:195-205, 1997; Brady and Sonne, Ale. Res. Health 23:263-271, 1999; Sinha, Psychopharmacol. 158:343-359, 2001; and Sinha et al., Psychopharmacol. 142:343-351, 1999), or simply the presentation of stress-related imagery (Sinha et al., Psychopharmacol. 158:343-359, 2000), have also been identified as potent events for provoking relapse to drug seeking in humans. Accordingly, the present invention contemplates two sets of effective doses: a dose required to treat addiction and another dose required to prevent recidivism (maintain abstinence; “abstinence dose,” hereinafter). In some embodiments, the abstinence dose may contain same amount of first agent and the second agent as the dose required to treat addiction. In some embodiments, the abstinence dose may contain same amount of first agent but higher amount of the second agent compared to the dose required to treat addiction. In some embodiments, the abstinence dose may contain same amount of first agent but lower amount of the second agent compared to the dose required to treat addiction. In some embodiments, the abstinence dose may contain lower amount of first agent but same amount of the second agent compared to the dose required to treat addiction. In some embodiments, the abstinence dose may contain higher amount of first agent but same amount of the second agent compared to the dose required to treat addiction. In some embodiments, the abstinence dose may contain lower amount of first agent and lower amount of the second agent compared to the dose required to treat addiction. In some embodiments, the abstinence dose may contain higher amount of first agent but lower amount of the second agent compared to the dose required to treat addiction. In some embodiments, the abstinence dose may contain higher amount of first agent and higher amount of the second agent compared to the dose required to treat addiction.

In some embodiments, any compound used in the method of the present invention, the therapeutically effective abstinence dose may be estimated initially from cell culture assays (e.g., assays designed to determine whether a nucleic acid, nucleic acid-based agent, or a protein such as an antibody inhibits (or stimulates) the expression or activity of the ligand or receptor it is intended to inhibit (or stimulate)). In some embodiments, any compound used in the method of the present invention, the therapeutically effective abstinence dose may be estimated based on experimental data in laboratory animals, including but not limited to such as rodents, rabbits, pigs, dogs and non-human primates. In some embodiments, any compound used in the method of the present invention, the therapeutically effective abstinence dose may be estimated based on one or more human clinical trials.

Routes of Administration:

The therapeutically effective doses of the first agent and the second agent may be administered using any medically acceptable mode of administration. Although the skilled artisan would contemplate any of the modes of administration known to one of ordinary skill, preferably the pharmacologic agent is administered according to the recommended mode of administration, for example, the mode of administration listed on the package insert of a commercially available agent.

In some embodiments, pharmaceutical compositions of the present invention may be formulated for administration by any route of administration, including, but not limited to, oral, injection or infusion, topical, intranasal, ocular, transmucosal, pulmonary, vaginal, rectal, parenteral, intradermal, subcutaneous, intramuscular, intravenous, intraosseous, intraperitoneal, intrathecal, epidural, intracardiac, intraarticular, intracavernous, intravitreal, intravaginal, intracervical, and inhalation routes. Dosage of the pharmaceutical compositions may vary by route of administration. Certain administration methods may include the step of administering the composition one or more times a day to obtain the desired therapeutic effect. The first agent and the second agent may be administered together or separately with same or different route of administration. Several possible embodiments of the invention have been described. Nevertheless, it will be understood that various modifications may be made without departing from the spirit and scope of the invention. Accordingly, other embodiments are within the scope of the following claims.

The amounts of the agents within a pharmaceutical preparation may be the same or different (e.g., the ratio of the first agent to the second can be at least or about 100:1; 90:1; 80:1; 75:1; 70:1; 65:1; 60:1; 55:1; 50:1; 45:1; 40:1; 35:1; 30:1; 25:1; 20:1; 15:1; 10:1; 9:1; 8:1; 7:1; 6:1; 5:1 ; 4:1: 3:1; 2:1; or about 1:1). For example, a composition can contain about 1 equivalent of oxazepam to about 25-50 equivalents of metyrapone; about 25-50 equivalents of ketoconazole to about 1 equivalent of alprazolam; about 25-50 equivalents of ketoconazole to about 1 equivalent of oxazepam; about 25-50 equivalent of metyrapone to about 1 equivalent of alprazolam; about 1 equivalent of muscimol to about 25-50 equivalents of CP-154,526; or about 1 equivalent of muscimol to about 25-50 equivalents of metyrapone. For example, a composition can contain about 1 equivalent of verucerfont to about 25-50 equivalents of any of escitalopram, metoprolol, ariprazole, buspirone, or clonidine; about 1 equivalent of pexacerfont to about 25-50 equivalents of any of escitalopram, metoprolol, ariprazole, buspirone, or clonidine; or about 1 equivalent of fluconazole to about 25-50 equivalents of any of escitalopram, metoprolol, ariprazole, buspirone, or clonidine. An equivalent can be a unit of weight (e.g., a milligram). The ratios can run differently, however, with the amount of the second agent exceeding the amount of the first agent (by, for example, the varying extent described here). The relative amounts of the active ingredients can also be expressed in terms of percentage. For example, relative to one another, the amount of the second agent can be at least or about 1-99% of the amount of the second agent. Where a third agent is included to inhibit the sympathetic nervous system, the relative amount of that agent can also vary with respect to the first and second agents. For example, relative to one another, the amount of the third agent can be at least or about 1-99% of the amount of the first or second agent. Where the third agent is included in a composition and/or used in a treatment regime, it may allow use of either the first and/or the second agent in an amount that is lower than predicted or that is required for efficacy in the absence of the third agent.

| Combination | | | Combination | | |
|---|---|---|---|---|---|
| No. | Compound 1 | Compound 2 | No. | Compound 1 | Compound 2 |
| 29 | verucerfont | escitalopram | 652 | verucerfont | metoprolol |
| 53 | verucerfont | ariprazole | 74 | verucerfont | buspirone |
| 72 | verucerfont | clonidine | 605 | pexacerfont | escitalopram |
| 664 | pexacerfont | metoprolol | 629 | pexacerfont | ariprazole |
| 650 | pexacerfont | buspirone | 648 | pexacerfont | clonidine |
| 557 | fluconazole | escitalopram | 663 | fluconazole | metoprolol |
| 581 | fluconazole | ariprazole | 602 | fluconazole | buspirone |
| 600 | fluconazole | clonidine | | | |

Third Agent

In one embodiment, a third agent is added to the combination of a first agent and a second agent, wherein the third agent is a modulator of pituitary targets, including but not limited to somatostatin analogs (e.g. somatostatin-14, octreotide, lanreotide, vapreotide, pasireotide and somatoprim), dopamine agonist, antidepressants or any of the compounds described as the first agent and the second agent. The third agent (i.e., the agent used in addition to the agent that targets the HPA axis and/or the agent that targets the prefrontal cortex) can also be an antidepressant, including any of the agents in the SSRI (selective serotonin reuptake inhibitor) class. Where either or both of the first and second agents are used in combination with a third agent that inhibits the sympathetic nervous system, the “third” agent can be a nucleic acid that inhibits the expression of a neurotransmitter or its cognate receptor within the sympathetic nervous system (e.g., the nucleic acid can inhibit the expression of a β adrenergic receptor).

Any of the compositions described herein can further include a third agent that inhibits activity in the sympathetic nervous system. Agents that inhibit the sympathetic nervous system include those known in the art as “beta blockers.” The third agent can be a beta blocker (e.g., sotalol, imolol, carteolol, carvedilol, nadolol, nadol/bendroflunetazide, propranolol, propranolol/HCTZ, betaxolol, penbutolol, metoprolol, labetalol, acebutolol, atenolol/HCTZ, atenolol, timolol/HCTZ, metoprolol, labetalol, pindolol, bisoprolol, bisoprolol/HCTZ, or esmolol), or other anxiolytic compound (e.g., an SSRI such as citalopram, escitalopram oxalate, fluvoxamine, paroxetine, fluoxetine, or sertraline). The anxiolytic compound or agent can also be an angiotensin II inhibitor (e.g., candasartan, eprosartan, irbesartan, losartan, telmisartan, or valsartan).

Any of the compositions described herein can further include a third agent that inhibits activity in the sympathetic nervous system. Agents that inhibit the sympathetic nervous system include those known in the art as “beta blockers.” The third agent can be a beta blocker (e.g., propanolol; bucindolol; carteolol; carvedilol; labetol; nadolol; oxprenolol; penbutolol; pindolol; sotalol; timolol; acebutolol; atenolol; betaxolol; bisoprolol; celiprolol; esmolol; metoprolol; nebivolol; butaxamine; ICI-118,551; and SR-59230A), or other anxiolytic compound (e.g., selective reuptake inhibitors (SSRIs); serotonin norepinephrine reuptake inhibitors (SNRIs); serotonin modulators and stimulators (SMSs); serotonin antagonists and reuptake inhibitors (SARIs); norepinephrine reuptake inhibitors (NRIs); tricyclic antidepressants (TCAs); tetracyclic antidepressants (TeCAs); monoamine oxidase inhibitors (MAOIs); norepinephrine dopamine reuptake inhibitors; agomelatine; bifemelane; tandospirone; and teniloxazine). The anxiolytic compound or agent can also be an angiotensin II inhibitor (e.g., candasartan, eprosartan, irbesartan, losartan, telmisartan, or valsartan).

Other suitable third agents include but are not limited to bromocriptine, cabergoline, somatostatin analogs (for example, Lanreotide®), Octreotide®, pegvisomant (Somavert®).

The pharmaceutical compositions, which are described further below, can include standard ingredients such as carriers and preservatives. The compositions can also include substances (e.g., a polyethylene glycol) to increase the solubility of the active ingredients. Typically, the active ingredients will account for a minority of the overall composition. For example, the first, second, and/or third agents can constitute about 1-50%) of the pharmaceutical composition (e.g., about 1-40%; 1-30%; 1-20%; 1-10%; 2-40%; 2-30%; 2-20%; 2-10%i; 2-5%; 3-40; 3-30%; 3-20%; 3-10%; 3-5%; 4-40%; 4-30%; 4-20%; 4-10%; 4-5%; 1-2%; 1-3%; 1-4%; 2-4%; 2-3%; or 3-4% of the pharmaceutical composition).

### EXAMPLES

### Example 1

Effects of low dose combination pharmacotherapy on cocaine, nicotine or methamphetamine self-administration in rats: The studies described here are designed to examine a combination pharmacotherapy, consistent with that described herein, for the treatment of addiction (more specifically, cocaine nicotine or methamphetamine abuse; hereinafter “addictive substance”). Using this approach, two compounds, which are believed to use divergent mechanisms of action to ultimately produce similar effects on the body's responses to stressors, are administered together at doses that are ineffective, or much less effective, for the treatment of addiction when administered alone. Adult male Wistar rats are trained under a multiple, alternating schedule of addictive substance and food self-administration. This schedule consists of alternating periods of addictive substance access and food reinforcement. In some instances, as described further below, three doses of addictive substance (For example, 0.125, 0.25, or 0.50 mg/kg/infusion in case of cocaine) are tested. Rats are also periodically trained with saline substitution (the invention may include or exclude any of the listed agents extinction) and food extinction during the same session.

These studies are designed to check whether that pretreatment with mitotane, aminoglutethimide, etomidate, and (R)-4-(6,7-dihydro-5H-pyrrolo[l ,2-c]imidazol-5-yl)-3-fluorobenzonitrile (LCI699 hereinafter), the benzodiazepines chlordiazepoxide, alprazolam and oxazepam, decrease addictive substance self-administration and the reinstatement of extinguished addictive substance seeking in rats.

### Example 2

Test Combinations: The contemplated combinations of drugs tested are one or more of: (1) LCI699 alone (2) LCI699 and oxazepam; (3) LCI699 and alprazolam; (4) LCI699 and chlordiazepoxide; (5) mitotane alone; (6) mitotane and oxazepam; (7) mitotane and alprazolam; (8) mitotane and chlordiazepoxide; (9) aminoglutethimide alone; (10) aminoglutethimide and oxazepam; (11) aminoglutethimide and alprazolam; (12) aminoglutethimide and chlordiazepoxide; (13) etomidate alone; (14) etomidate and oxazepam; (15) etomidate and alprazolam; (16) etomidate and chlordiazepoxide; (17) ketoconazole alone; (18) ketoconazole and chlordiazepoxide; (19) ketoconazole and oxazepam; (20) ketoconazole and alprazolam; (21) 2S,4R enantiomer of ketoconazole alone; (22) 2S,4R enantiomer of ketoconazole and chlordiazepoxide; (23) 2S,4R enantiomer of ketoconazole and oxazepam; (24) 2S,4R enantiomer of ketoconazole and alprazolam; (25) oxazepam alone; (26) alprazolam alone and (27) chlordiazepoxide alone. “the test combinations hereinafter)”. The test combinations consist of at least one compound of the class first agent (e.g., LCI699, mitotane, aminoglutethimide, etomidate, enantiomer of ketoconazole and 2S,4R enantiomer of ketoconazole) and/or one compound of the class second agent (e.g. oxazepam, alprazolam and chlordiazepoxide). In some experiments, the drugs are used at doses that are below the below the normally effective doses of the first agent alone or the second agent alone for the treatment of addiction; and an the experiments are designed to look for additive or synergistic effects.

Additional contemplated combinations of drugs tested are one or more of:

| Combination | | | Combination | | |
|---|---|---|---|---|---|
| No. | Compound 1 | Compound 2 | No. | Compound 1 | Compound 2 |
| 28 | verucerfont | citalopram | 29 | verucerfont | escitalopram |
| 30 | verucerfont | paroxetine | 31 | verucerfont | fluoxetine |
| 32 | verucerfont | fluvoxamine | 33 | verucerfont | sertraline |
| 34 | verucerfont | desvenlafaxine | 35 | verucerfont | duloxetine |
| 36 | verucerfont | levomilnacipran | 37 | verucerfont | milnacipran |
| 38 | verucerfont | tofenacin | 39 | verucerfont | venlafaxine |
| 40 | verucerfont | vilazodone | 41 | verucerfont | vortioxetine |
| 42 | verucerfont | bupropion | 43 | verucerfont | agomelatine |
| 44 | verucerfont | bifemelane | 45 | verucerfont | tandospirone |
| 46 | verucerfont | teniloxazine | 47 | verucerfont | bucindolol |
| 48 | verucerfont | oxprenolol | 49 | verucerfont | celiprolol |
| 50 | verucerfont | nebivolol | 51 | verucerfont | amisulpride |
| 52 | verucerfont | amoxapine | 53 | verucerfont | arpiprazole |
| 54 | verucerfont | asenapine | 55 | verucerfont | cariprazine |
| 56 | verucerfont | clozapine | 57 | verucerfont | blonaserin |
| 58 | verucerfont | iloperidone | 59 | verucerfont | lurasidone |
| 60 | verucerfont | melperone | 61 | verucerfont | nemonapride |
| 62 | verucerfont | olanzapine | 63 | verucerfont | paliperidone |
| 64 | verucerfont | perospirone | 65 | verucerfont | quetiapine |
| 66 | verucerfont | remoxapride | 67 | verucerfont | risperidone |
| 68 | verucerfont | sertindole | 69 | verucerfont | trimipramine |
| 70 | verucerfont | ziprasidone | 71 | verucerfont | zotepine |
| 72 | verucerfont | clonidine | 73 | verucerfont | guanfacine |
| 74 | verucerfont | buspirone | 75 | verucerfont | tandospirone |
| 76 | LWH-234 | citalopram | 77 | LWH-234 | escitalopram |
| 78 | LWH-234 | paroxetine | 79 | LWH-234 | fluoxetine |
| 80 | LWH-234 | fluvoxamine | 81 | LWH-234 | sertraline |
| 82 | LWH-234 | desvenlafaxine | 83 | LWH-234 | duloxetine |
| 84 | LWH-234 | levomilnacipran | 85 | LWH-234 | milnacipran |
| 86 | LWH-234 | tofenacin | 87 | LWH-234 | venlafaxine |
| 88 | LWH-234 | vilazodone | 89 | LWH-234 | vortioxetine |
| 90 | LWH-234 | bupropion | 91 | LWH-234 | agomelatine |
| 92 | LWH-234 | bifemelane | 93 | LWH-234 | tandospirone |
| 94 | LWH-234 | teniloxazine | 95 | LWH-234 | bucindolol |
| 96 | LWH-234 | oxprenolol | 97 | LWH-234 | celiprolol |
| 98 | LWH-234 | nebivolol | 99 | LWH-234 | amisulpride |
| 100 | LWH-234 | amoxapine | 101 | LWH-234 | arpiprazole |
| 102 | LWH-234 | asenapine | 103 | LWH-234 | cariprazine |
| 104 | LWH-234 | clozapine | 105 | LWH-234 | blonaserin |
| 106 | LWH-234 | iloperidone | 107 | LWH-234 | lurasidone |
| 108 | LWH-234 | melperone | 109 | LWH-234 | nemonapride |
| 110 | LWH-234 | olanzapine | 111 | LWH-234 | paliperidone |
| 112 | LWH-234 | perospirone | 113 | LWH-234 | quetiapine |
| 114 | LWH-234 | remoxapride | 115 | LWH-234 | risperidone |
| 116 | LWH-234 | sertindole | 117 | LWH-234 | trimipramine |
| 118 | LWH-234 | ziprasidone | 119 | LWH-234 | zotepine |
| 120 | LWH-234 | clonidine | 121 | LWH-234 | guanfacine |
| 122 | LWH-234 | buspirone | 123 | LWH-234 | tandospirone |
| 124 | R-121,919 | citalopram | 125 | R-121,919 | escitalopram |
| 126 | R-121,919 | paroxetine | 127 | R-121,919 | fluoxetine |
| 128 | R-121,919 | fluvoxamine | 129 | R-121,919 | sertraline |
| 130 | R-121,919 | desvenlafaxine | 131 | R-121,919 | duloxetine |
| 132 | R-121,919 | levomilnacipran | 133 | R-121,919 | milnacipran |
| 134 | R-121,919 | tofenacin | 135 | R-121,919 | venlafaxine |
| 136 | R-121,919 | vilazodone | 137 | R-121,919 | vortioxetine |
| 138 | R-121,919 | bupropion | 139 | R-121,919 | agomelatine |
| 140 | R-121,919 | bifemelane | 141 | R-121,919 | tandospirone |
| 142 | R-121,919 | teniloxazine | 143 | R-121,919 | bucindolol |
| 144 | R-121,919 | oxprenolol | 145 | R-121,919 | celiprolol |
| 146 | R-121,919 | nebivolol | 147 | R-121,919 | amisulpride |
| 148 | R-121,919 | amoxapine | 149 | R-121,919 | arpiprazole |
| 150 | R-121,919 | asenapine | 151 | R-121,919 | cariprazine |
| 152 | R-121,919 | clozapine | 153 | R-121,919 | blonaserin |
| 154 | R-121,919 | iloperidone | 155 | R-121,919 | lurasidone |
| 156 | R-121,919 | melperone | 157 | R-121,919 | nemonapride |
| 158 | R-121,919 | olanzapine | 159 | R-121,919 | paliperidone |
| 160 | R-121,919 | perospirone | 161 | R-121,919 | quetiapine |
| 162 | R-121,919 | remoxapride | 163 | R-121,919 | risperidone |
| 164 | R-121,919 | sertindole | 165 | R-121,919 | trimipramine |
| 166 | R-121,919 | ziprasidone | 167 | R-121,919 | zotepine |
| 168 | R-121,919 | clonidine | 169 | R-121,919 | guanfacine |
| 170 | R-121,919 | buspirone | 171 | R-121,919 | tandospirone |
| 172 | bromocriptine | citalopram | 173 | bromocriptine | escitalopram |
| 174 | bromocriptine | paroxetine | 175 | bromocriptine | fluoxetine |
| 176 | bromocriptine | fluvoxamine | 177 | bromocriptine | sertraline |
| 178 | bromocriptine | desvenlafaxine | 179 | bromocriptine | duloxetine |
| 180 | bromocriptine | levomilnacipran | 181 | bromocriptine | milnacipran |
| 182 | bromocriptine | tofenacin | 183 | bromocriptine | venlafaxine |
| 184 | bromocriptine | vilazodone | 185 | bromocriptine | vortioxetine |
| 186 | bromocriptine | bupropion | 187 | bromocriptine | agomelatine |
| 188 | bromocriptine | bifemelane | 189 | bromocriptine | tandospirone |
| 190 | bromocriptine | teniloxazine | 191 | bromocriptine | bucindolol |
| 192 | bromocriptine | oxprenolol | 193 | bromocriptine | celiprolol |
| 194 | bromocriptine | nebivolol | 195 | bromocriptine | amisulpride |
| 196 | bromocriptine | amoxapine | 197 | bromocriptine | arpiprazole |
| 198 | bromocriptine | asenapine | 199 | bromocriptine | cariprazine |
| 200 | bromocriptine | clozapine | 201 | bromocriptine | blonaserin |
| 202 | bromocriptine | iloperidone | 203 | bromocriptine | lurasidone |
| 204 | bromocriptine | melperone | 205 | bromocriptine | nemonapride |
| 206 | bromocriptine | olanzapine | 207 | bromocriptine | paliperidone |
| 208 | bromocriptine | perospirone | 209 | bromocriptine | quetiapine |
| 210 | bromocriptine | remoxapride | 211 | bromocriptine | risperidone |
| 212 | bromocriptine | sertindole | 213 | bromocriptine | trimipramine |
| 214 | bromocriptine | ziprasidone | 215 | bromocriptine | zotepine |
| 216 | bromocriptine | clonidine | 217 | bromocriptine | guanfacine |
| 218 | bromocriptine | buspirone | 219 | bromocriptine | tandospirone |
| 220 | cabergoline | citalopram | 221 | cabergoline | escitalopram |
| 222 | cabergoline | paroxetine | 223 | cabergoline | fluoxetine |
| 224 | cabergoline | fluvoxamine | 225 | cabergoline | sertraline |
| 226 | cabergoline | desvenlafaxine | 227 | cabergoline | duloxetine |
| 228 | cabergoline | levomilnacipran | 229 | cabergoline | milnacipran |
| 230 | cabergoline | tofenacin | 231 | cabergoline | venlafaxine |
| 232 | cabergoline | vilazodone | 233 | cabergoline | vortioxetine |
| 234 | cabergoline | bupropion | 235 | cabergoline | agomelatine |
| 236 | cabergoline | bifemelane | 237 | cabergoline | tandospirone |
| 238 | cabergoline | teniloxazine | 239 | cabergoline | bucindolol |
| 240 | cabergoline | oxprenolol | 241 | cabergoline | celiprolol |
| 242 | cabergoline | nebivolol | 243 | cabergoline | amisulpride |
| 244 | cabergoline | amoxapine | 245 | cabergoline | arpiprazole |
| 246 | cabergoline | asenapine | 247 | cabergoline | cariprazine |
| 248 | cabergoline | clozapine | 249 | cabergoline | blonaserin |
| 250 | cabergoline | iloperidone | 251 | cabergoline | lurasidone |
| 252 | cabergoline | melperone | 253 | cabergoline | nemonapride |
| 254 | cabergoline | olanzapine | 255 | cabergoline | paliperidone |
| 256 | cabergoline | perospirone | 257 | cabergoline | quetiapine |
| 258 | cabergoline | remoxapride | 259 | cabergoline | risperidone |
| 260 | cabergoline | sertindole | 261 | cabergoline | trimipramine |
| 262 | cabergoline | ziprasidone | 263 | cabergoline | zotepine |
| 264 | cabergoline | clonidine | 265 | cabergoline | guanfacine |
| 266 | cabergoline | buspirone | 267 | cabergoline | tandospirone |
| 268 | octreotide | citalopram | 269 | octreotide | escitalopram |
| 270 | octreotide | paroxetine | 271 | octreotide | fluoxetine |
| 272 | octreotide | fluvoxamine | 273 | octreotide | sertraline |
| 274 | octreotide | desvenlafaxine | 275 | octreotide | duloxetine |
| 276 | octreotide | levomilnacipran | 277 | octreotide | milnacipran |
| 278 | octreotide | tofenacin | 279 | octreotide | venlafaxine |
| 280 | octreotide | vilazodone | 281 | octreotide | vortioxetine |
| 282 | octreotide | bupropion | 283 | octreotide | agomelatine |
| 284 | octreotide | bifemelane | 285 | octreotide | tandospirone |
| 286 | octreotide | teniloxazine | 287 | octreotide | bucindolol |
| 288 | octreotide | oxprenolol | 289 | octreotide | celiprolol |
| 290 | octreotide | nebivolol | 291 | octreotide | amisulpride |
| 292 | octreotide | amoxapine | 293 | octreotide | arpiprazole |
| 294 | octreotide | asenapine | 295 | octreotide | cariprazine |
| 296 | octreotide | clozapine | 297 | octreotide | blonaserin |
| 298 | octreotide | iloperidone | 299 | octreotide | lurasidone |
| 300 | octreotide | melperone | 301 | octreotide | nemonapride |
| 302 | octreotide | olanzapine | 303 | octreotide | paliperidone |
| 304 | octreotide | perospirone | 305 | octreotide | quetiapine |
| 306 | octreotide | remoxapride | 307 | octreotide | risperidone |
| 308 | octreotide | sertindole | 309 | octreotide | trimipramine |
| 310 | octreotide | ziprasidone | 311 | octreotide | zotepine |
| 312 | octreotide | clonidine | 313 | octreotide | guanfacine |
| 314 | octreotide | buspirone | 315 | octreotide | tandospirone |
| 316 | pasireotide | citalopram | 317 | pasireotide | escitalopram |
| 318 | pasireotide | paroxetine | 319 | pasireotide | fluoxetine |
| 320 | pasireotide | fluvoxamine | 321 | pasireotide | sertraline |
| 322 | pasireotide | desvenlafaxine | 323 | pasireotide | duloxetine |
| 324 | pasireotide | levomilnacipran | 325 | pasireotide | milnacipran |
| 326 | pasireotide | tofenacin | 327 | pasireotide | venlafaxine |
| 328 | pasireotide | vilazodone | 329 | pasireotide | vortioxetine |
| 330 | pasireotide | bupropion | 331 | pasireotide | agomelatine |
| 332 | pasireotide | bifemelane | 333 | pasireotide | tandospirone |
| 334 | pasireotide | teniloxazine | 335 | pasireotide | bucindolol |
| 336 | pasireotide | oxprenolol | 337 | pasireotide | celiprolol |
| 338 | pasireotide | nebivolol | 339 | pasireotide | amisulpride |
| 340 | pasireotide | amoxapine | 341 | pasireotide | arpiprazole |
| 342 | pasireotide | asenapine | 343 | pasireotide | cariprazine |
| 344 | pasireotide | clozapine | 345 | pasireotide | blonaserin |
| 346 | pasireotide | iloperidone | 347 | pasireotide | lurasidone |
| 348 | pasireotide | melperone | 349 | pasireotide | nemonapride |
| 350 | pasireotide | olanzapine | 351 | pasireotide | paliperidone |
| 352 | pasireotide | perospirone | 353 | pasireotide | quetiapine |
| 354 | pasireotide | remoxapride | 355 | pasireotide | risperidone |
| 356 | pasireotide | sertindole | 357 | pasireotide | trimipramine |
| 358 | pasireotide | ziprasidone | 359 | pasireotide | zotepine |
| 360 | pasireotide | clonidine | 361 | pasireotide | guanfacine |
| 362 | pasireotide | buspirone | 363 | pasireotide | tandospirone |
| 364 | retinoic acid | citalopram | 365 | retinoic acid | escitalopram |
| 366 | retinoic acid | paroxetine | 367 | retinoic acid | fluoxetine |
| 368 | retinoic acid | fluvoxamine | 369 | retinoic acid | sertraline |
| 370 | retinoic acid | desvenlafaxine | 371 | retinoic acid | duloxetine |
| 372 | retinoic acid | levomilnacipran | 373 | retinoic acid | milnacipran |
| 374 | retinoic acid | tofenacin | 375 | retinoic acid | venlafaxine |
| 376 | retinoic acid | vilazodone | 377 | retinoic acid | vortioxetine |
| 378 | retinoic acid | bupropion | 379 | retinoic acid | agomelatine |
| 380 | retinoic acid | bifemelane | 381 | retinoic acid | tandospirone |
| 382 | retinoic acid | teniloxazine | 383 | retinoic acid | bucindolol |
| 384 | retinoic acid | oxprenolol | 385 | retinoic acid | celiprolol |
| 386 | retinoic acid | nebivolol | 387 | retinoic acid | amisulpride |
| 388 | retinoic acid | amoxapine | 389 | retinoic acid | arpiprazole |
| 390 | retinoic acid | asenapine | 391 | retinoic acid | cariprazine |
| 392 | retinoic acid | clozapine | 393 | retinoic acid | blonaserin |
| 394 | retinoic acid | iloperidone | 395 | retinoic acid | lurasidone |
| 396 | retinoic acid | melperone | 397 | retinoic acid | nemonapride |
| 398 | retinoic acid | olanzapine | 399 | retinoic acid | paliperidone |
| 400 | retinoic acid | perospirone | 401 | retinoic acid | quetiapine |
| 402 | retinoic acid | remoxapride | 403 | retinoic acid | risperidone |
| 404 | retinoic acid | sertindole | 405 | retinoic acid | trimipramine |
| 406 | retinoic acid | ziprasidone | 407 | retinoic acid | zotepine |
| 408 | retinoic acid | clonidine | 409 | retinoic acid | guanfacine |
| 410 | retinoic acid | buspirone | 411 | retinoic acid | tandospirone |
| 412 | cyproheptadine | citalopram | 413 | cyproheptadine | escitalopram |
| 414 | cyproheptadine | paroxetine | 415 | cyproheptadine | fluoxetine |
| 416 | cyproheptadine | fluvoxamine | 417 | cyproheptadine | sertraline |
| 418 | cyproheptadine | desvenlafaxine | 419 | cyproheptadine | duloxetine |
| 420 | cyproheptadine | levomilnacipran | 421 | cyproheptadine | milnacipran |
| 422 | cyproheptadine | tofenacin | 423 | cyproheptadine | venlafaxine |
| 424 | cyproheptadine | vilazodone | 425 | cyproheptadine | vortioxetine |
| 426 | cyproheptadine | bupropion | 427 | cyproheptadine | agomelatine |
| 428 | cyproheptadine | bifemelane | 429 | cyproheptadine | tandospirone |
| 430 | cyproheptadine | teniloxazine | 431 | cyproheptadine | bucindolol |
| 432 | cyproheptadine | oxprenolol | 433 | cyproheptadine | celiprolol |
| 434 | cyproheptadine | nebivolol | 435 | cyproheptadine | amisulpride |
| 436 | cyproheptadine | amoxapine | 437 | cyproheptadine | arpiprazole |
| 438 | cyproheptadine | asenapine | 439 | cyproheptadine | cariprazine |
| 440 | cyproheptadine | clozapine | 441 | cyproheptadine | blonaserin |
| 442 | cyproheptadine | iloperidone | 443 | cyproheptadine | lurasidone |
| 444 | cyproheptadine | melperone | 445 | cyproheptadine | nemonapride |
| 446 | cyproheptadine | olanzapine | 447 | cyproheptadine | paliperidone |
| 448 | cyproheptadine | perospirone | 449 | cyproheptadine | quetiapine |
| 450 | cyproheptadine | remoxapride | 451 | cyproheptadine | risperidone |
| 452 | cyproheptadine | sertindole | 453 | cyproheptadine | trimipramine |
| 454 | cyproheptadine | ziprasidone | 455 | cyproheptadine | zotepine |
| 456 | cyproheptadine | clonidine | 457 | cyproheptadine | guanfacine |
| 458 | cyproheptadine | buspirone | 459 | cyproheptadine | tandospirone |
| 460 | mifepristone | citalopram | 461 | mifepristone | escitalopram |
| 462 | mifepristone | paroxetine | 463 | mifepristone | fluoxetine |
| 464 | mifepristone | fluvoxamine | 465 | mifepristone | sertraline |
| 466 | mifepristone | desvenlafaxine | 467 | mifepristone | duloxetine |
| 468 | mifepristone | levomilnacipran | 469 | mifepristone | milnacipran |
| 470 | mifepristone | tofenacin | 471 | mifepristone | venlafaxine |
| 472 | mifepristone | vilazodone | 473 | mifepristone | vortioxetine |
| 474 | mifepristone | bupropion | 475 | mifepristone | agomelatine |
| 476 | mifepristone | bifemelane | 477 | mifepristone | tandospirone |
| 478 | mifepristone | teniloxazine | 479 | mifepristone | bucindolol |
| 480 | mifepristone | oxprenolol | 481 | mifepristone | celiprolol |
| 482 | mifepristone | nebivolol | 483 | mifepristone | amisulpride |
| 484 | mifepristone | amoxapine | 485 | mifepristone | arpiprazole |
| 486 | mifepristone | asenapine | 487 | mifepristone | cariprazine |
| 488 | mifepristone | clozapine | 489 | mifepristone | blonaserin |
| 490 | mifepristone | iloperidone | 491 | mifepristone | lurasidone |
| 492 | mifepristone | melperone | 493 | mifepristone | nemonapride |
| 494 | mifepristone | olanzapine | 495 | mifepristone | paliperidone |
| 496 | mifepristone | perospirone | 497 | mifepristone | quetiapine |
| 498 | mifepristone | remoxapride | 499 | mifepristone | risperidone |
| 500 | mifepristone | sertindole | 501 | mifepristone | trimipramine |
| 502 | mifepristone | ziprasidone | 503 | mifepristone | zotepine |
| 504 | mifepristone | clonidine | 505 | mifepristone | guanfacine |
| 506 | mifepristone | buspirone | 507 | mifepristone | tandospirone |
| 508 | cytadren | citalopram | 509 | cytadren | escitalopram |
| 510 | cytadren | paroxetine | 511 | cytadren | fluoxetine |
| 512 | cytadren | fluvoxamine | 513 | cytadren | sertraline |
| 514 | cytadren | desvenlafaxine | 515 | cytadren | duloxetine |
| 516 | cytadren | levomilnacipran | 517 | cytadren | milnacipran |
| 518 | cytadren | tofenacin | 519 | cytadren | venlafaxine |
| 520 | cytadren | vilazodone | 521 | cytadren | vortioxetine |
| 522 | cytadren | bupropion | 523 | cytadren | agomelatine |
| 524 | cytadren | bifemelane | 525 | cytadren | tandospirone |
| 526 | cytadren | teniloxazine | 527 | cytadren | bucindolol |
| 528 | cytadren | oxprenolol | 529 | cytadren | celiprolol |
| 530 | cytadren | nebivolol | 531 | cytadren | amisulpride |
| 532 | cytadren | amoxapine | 533 | cytadren | arpiprazole |
| 534 | cytadren | asenapine | 535 | cytadren | cariprazine |
| 536 | cytadren | clozapine | 537 | cytadren | blonaserin |
| 538 | cytadren | iloperidone | 539 | cytadren | lurasidone |
| 540 | cytadren | melperone | 541 | cytadren | nemonapride |
| 542 | cytadren | olanzapine | 543 | cytadren | paliperidone |
| 544 | cytadren | perospirone | 545 | cytadren | quetiapine |
| 546 | cytadren | remoxapride | 547 | cytadren | risperidone |
| 548 | cytadren | sertindole | 549 | cytadren | trimipramine |
| 550 | cytadren | ziprasidone | 551 | cytadren | zotepine |
| 552 | cytadren | clonidine | 553 | cytadren | guanfacine |
| 554 | cytadren | buspirone | 555 | cytadren | tandospirone |
| 556 | fluconazole | citalopram | 557 | fluconazole | escitalopram |
| 558 | fluconazole | paroxetine | 559 | fluconazole | fluoxetine |
| 560 | fluconazole | fluvoxamine | 561 | fluconazole | sertraline |
| 562 | fluconazole | desvenlafaxine | 563 | fluconazole | duloxetine |
| 564 | fluconazole | levomilnacipran | 565 | fluconazole | milnacipran |
| 566 | fluconazole | tofenacin | 567 | fluconazole | venlafaxine |
| 568 | fluconazole | vilazodone | 569 | fluconazole | vortioxetine |
| 570 | fluconazole | bupropion | 571 | fluconazole | agomelatine |
| 572 | fluconazole | bifemelane | 573 | fluconazole | tandospirone |
| 574 | fluconazole | teniloxazine | 575 | fluconazole | bucindolol |
| 576 | fluconazole | oxprenolol | 577 | fluconazole | celiprolol |
| 578 | fluconazole | nebivolol | 579 | fluconazole | amisulpride |
| 580 | fluconazole | amoxapine | 581 | fluconazole | arpiprazole |
| 582 | fluconazole | asenapine | 583 | fluconazole | cariprazine |
| 584 | fluconazole | clozapine | 585 | fluconazole | blonaserin |
| 586 | fluconazole | iloperidone | 587 | fluconazole | lurasidone |
| 588 | fluconazole | melperone | 589 | fluconazole | nemonapride |
| 290 | fluconazole | olanzapine | 591 | fluconazole | paliperidone |
| 592 | fluconazole | perospirone | 593 | fluconazole | quetiapine |
| 594 | fluconazole | remoxapride | 595 | fluconazole | risperidone |
| 596 | fluconazole | sertindole | 597 | fluconazole | trimipramine |
| 598 | fluconazole | ziprasidone | 599 | fluconazole | zotepine |
| 600 | fluconazole | clonidine | 601 | fluconazole | guanfacine |
| 602 | fluconazole | buspirone | 603 | fluconazole | tandospirone |
| 604 | pexacerfont | citalopram | 605 | pexacerfont | escitalopram |
| 606 | pexacerfont | paroxetine | 607 | pexacerfont | fluoxetine |
| 608 | pexacerfont | fluvoxamine | 609 | pexacerfont | sertraline |
| 610 | pexacerfont | desvenlafaxine | 611 | pexacerfont | duloxetine |
| 612 | pexacerfont | levomilnacipran | 613 | pexacerfont | milnacipran |
| 614 | pexacerfont | tofenacin | 615 | pexacerfont | venlafaxine |
| 616 | pexacerfont | vilazodone | 617 | pexacerfont | vortioxetine |
| 618 | pexacerfont | bupropion | 619 | pexacerfont | agomelatine |
| 620 | pexacerfont | bifemelane | 621 | pexacerfont | tandospirone |
| 622 | pexacerfont | teniloxazine | 623 | pexacerfont | bucindolol |
| 624 | pexacerfont | oxprenolol | 625 | pexacerfont | celiprolol |
| 626 | pexacerfont | nebivolol | 627 | pexacerfont | amisulpride |
| 628 | pexacerfont | amoxapine | 629 | pexacerfont | arpiprazole |
| 630 | pexacerfont | asenapine | 631 | pexacerfont | cariprazine |
| 632 | pexacerfont | clozapine | 633 | pexacerfont | blonaserin |
| 634 | pexacerfont | iloperidone | 635 | pexacerfont | lurasidone |
| 636 | pexacerfont | melperone | 637 | pexacerfont | nemonapride |
| 638 | pexacerfont | olanzapine | 639 | pexacerfont | paliperidone |
| 640 | pexacerfont | perospirone | 641 | pexacerfont | quetiapine |
| 642 | pexacerfont | remoxapride | 643 | pexacerfont | risperidone |
| 644 | pexacerfont | sertindole | 645 | pexacerfont | trimipramine |
| 646 | pexacerfont | ziprasidone | 647 | pexacerfont | zotepine |
| 648 | pexacerfont | clonidine | 649 | pexacerfont | guanfacine |
| 650 | pexacerfont | buspirone | 651 | pexacerfont | tandospirone |
| 652 | verucerfont | metoprolol | 653 | LWH-234 | metoprolol |
| 654 | R-121,919 | metoprolol | 655 | bromocriptine | metoprolol |
| 656 | cabergoline | metoprolol | 657 | octreotide | metoprolol |
| 658 | pasireotide | metoprolol | 659 | retinoic acid | metoprolol |
| 660 | cyproheptadine | metoprolol | 661 | mifepristone | metoprolol |
| 662 | cytadren | metoprolol | 663 | fluconazole | metoprolol |
| 664 | pexacerfont | metoprolol | | | |

The contemplated doses of LCI699, mitotane, aminoglutethimide, etomidate, oxazepam, alprazolam and chlordiazepoxide are independently selected and range from 0.0001 mg/Kg to about 1 g/Kg per day. In some experiments the drugs are formulated as a composition for injection. The combinations are either co-formulated or separately formulated. In other embodiments, the second agent is co-administered separately using same or different route, such as one of the routes of administration described above.

The contemplated doses of verucerfont; pexacerfont; LWH-234; R-121,919; bromocriptine; cabergoline; octreotide; pasireotide; retinoic acid; cyproheptadine; mifepristone; cytadren; fluconazole; citalopram; escitalopram; paroxetine; fluoxetine; fluvoxamine; sertraline; desvenlafaxine; duloxetine; levomilnacipran; milnacipran; tofenacin; venlafaxine; vilazodone; vortioxetine; bupropion; agomelatine; bifemelane; tandospirone; teniloxazine; bucindolol; metoprolol; oxprenolol; celiprolol; nebivolol; amisulpride; amoxapine; arpiprazole; asenapine; cariprazine; clozapine; blonaserin; iloperidone; lurasidone; melperone; nemonapride; olanzapine; paliperidone; perospirone; quetiapine; remoxapride; risperidone; sertindole; trimipramine; ziprasidone; zotepine; clonidine; guanfacine; buspirone; and tandospirone are independently selected and range from 0.0001 mg/Kg to about 1 g/Kg per day. In some experiments the drugs are formulated as a composition for injection. The combinations are either co-formulated or separately formulated. In other embodiments, the second agent is co-administered separately using same or different route, such as one of the routes of administration described above.

The combined dosage form(s) described above are assessed for efficacy in treating cocaine use disorder, as well as for safety. One exemplary study is a randomized, double-blind, parallel-group, abstinence-initiation study in subjects with moderate to severe cocaine use disorder. Subjects who meet screening criteria are randomized (1:1:1) to 1 of 2 combination dose groups, individual dose groups or placebo (n=10³/arm). Oral doses of each of the drug compositions or placebo are selected on earlier animal and human data relating to the specific active pharmaceutical ingredients. Randomization is stratified by cocaine use frequency (>10 or ≤10 days of use over the 28 days prior to consent). Following an 11 week drug treatment period, subjects are followed for another week for any signs of withdrawal. All subjects also receive weekly cognitive behavioral therapy (CBT) adapted for treating substance use disorders.

Urine is also assessed three times per week by study personnel using onsite collected samples. Other methods are employed to optimize adherence to study medication and protocol activities, including a contingency management system and plasma drug concentrations. They may also include riboflavin or another tracer in the study drug capsules, Medication Events Monitoring System (MEMS) devices and other approaches. Methods such as these have been shown to reduce dropouts and increase adherence to study drug and study procedures in similar trials. The study is conducted at 10 U.S. study centers with extensive experience in substance use disorder studies.

The Primary Efficacy Measure (outcome measure) is continuous abstinence from cocaine use over the final 3 weeks of the drug treatment phase as assessed by urine BE-confirmed self-reports.

The Secondary Efficacy Measure includes weekly cocaine non-use days (confirmed or disproved by urine BE levels), quantitative measurements of urine cocaine and urine BE, the Cocaine Craving Questionnaire-Brief (CCQ-B), the Hospital Anxiety and Depression Scale (HADS), the Addictions Severity Index (ASI), the Sheehan Disability Scale (SDS) and the Quality of Life Enjoyment and Satisfaction Questionnaire (QLESQ). Subject retention and medication adherence are also characterized.

Safety measures include assessing vital signs, clinical laboratory tests, physical exams, 12-lead ECGs, the S-STS and assessment of AEs.

Measurement of cortisol and ACTS: Cortisol levels, along with symptomatic screening for adrenal insufficiency using the AIRC is used to exclude subjects who have suspected pre-existing adrenal insufficiency at screening. Serum cortisol and ACTH is measured at time intervals based on the results from the Phase 1 study and SA1. Sampling occurs at approximately 10:00 AM. Criteria for stopping study medication in response to low cortisol levels is refined based on results from any related Phase 1 study and SA1.

Drug concentration measurements: Blood is collected for measurement of active pharmaceutical agent as well as metabolites of the agent in plasma at weeks 2, 5, 8 and 11. Sampling times are based on results from related studies, such as Phase 1 or SA1 studies.

Abbreviated Inclusion Criteria: Males and non-pregnant, non-lactating females age 21-65 with moderate-severe cocaine use disorder as defined by the DSM-5, who are seeking treating, have at least 1 urine screen positive (>300 ng/mL) for BE during the 2-week screening period, and have at least 1 during screen negative for BE following the last positive screen and prior to randomization. No clinically abnormal physical findings at the screening examination are permissible; normal or clinically acceptable screening ECG; normal BP and heart rate; normal cortisol and ACTH levels and assessed by investigator as not having adrenal insufficiency by review of symptoms, clinical labs and clinical findings; BMI>18.5 and <35.

Abbreviated Exclusion Criteria: History of hypersensitivity to or medically significant averse events associated with cocaine or one or both active pharmaceutical agents. Treatment with an investigational drug or biologic within the 60 days preceding the randomization visit or plans to take another investigational drug or biologic within 30 days of study completion (including the follow-up visit. Lifetime history of psychotic (e.g., schizophrenia, schizophrenic disorder) or bipolar disorder that is not secondary to substance use, as defined in DSM-5; primary major mood disorder or anxiety disorder (e.g., major depression) within the past 5 years; a mental illness that requires or may require ongoing pharmacologic or other somatic treatment during participation in this study; and suicidal ideation.

Other abbreviated exclusion criteria include: enrollment in opiate substitution program within the 2 months prior to screening; subjects who require detoxification from alcohol, opiates, or sedative-hypnotic drugs; subjects with substance use disorders other than cocaine, marijuana, nicotine, or alcohol; subjects under a court order for cocaine use disorder treatment; positive during drug drug screen for opiates, benzodiazepines, barbituates or related CNS depressants, or amphetamines or related stimulants (other than cocaine); lifetime history of benzodiazepine dependence. Additional criteria include: history of adrenal insufficiency or other adrenal, pituitary, or hypothalamic disease; seizures or traumatic brain injury; neurologic or neuromuscular disease; history of clinically significant hypotension or cardiovascular disease; other severe, acute, or chronic medical or psychiatric condition or laboratory abnormality that may increase the risks associated with study participation or investigational product administration that may interfere with the interpretation of study results or, in the judgment of the investigator or sponsor, would make the subject inappropriate for entry into this study.

Allowed Prior and Concomitant Therapy:

Because this is a study of a CNS disorder, CNS-acting concomitant medication are not allowed. Monotherapy antidepressants (e.g., SSRIs, SNRIs) may be allowed if taken at a stable dose for at least 4 weeks prior to enrollment in the study, and the dose is not expected to change for the duration of the study. Other cortisol synthesis inhibitors (e.g., ketoconazole) are not allowed.

Analysis:

All outcomes are analyzed using appropriate statistical methods for the ITT population, i.e., all subjects who have taken at least one study medication dose are included in safety analyses, and all subjects who have taken at least one study medication dose and have at least one post-baseline efficacy assessment are included in efficacy analyses. Missing data is imputed using the baseline observation carried forward (BOCF) methodology favored by the FDA Division of Anesthesia, Analgesia and Addiction Products, although last observation carried forward (LOCF) and mixed model repeated measures (MMRM) analyses are conducted as sensitivity analyses. Data is summarized by treatment condition. Descriptive statistics generally include the mean, standard deviation, minima and maxima for continuous data, and frequencies/percentages for categorical data.

Sample Size Rationale:

Approximately 309 subjects with moderate-severe Cocaine Use Disorder (103 per arm) are randomized into the treatment phase of the study. Allowing for 20% dropouts, this provides 90% power to detect a difference in abstinence rates of 10% in the placebo arm and 30% in the effective dose arm, with alpha=0.05, two-tailed. A placebo abstinence rate of 10% is comparable to that seen in recent studies of roughly similar design and an approximately 30% abstinence treatment rate in an experimental arm is a clinically significant improvement. For a study of this design and duration, a dropout rate of approximately 20% is consistent with similar trials.

Results and Interpretation:

A 10% to 30% increase in abstinence in subjects during the last three weeks of treatment indicates a statistically significant difference and is indicative of efficacy for tretatment of subjects with moderate to severe cocaine use disorder, with as much as 10% abstinence associated with placebo. Secondary outcomes typically support these findings.

### Example 3

Training to self-administer cocaine: In this experiment, rats are exposed to alternating 15-minute periods of access to cocaine self-administration and food reinforcement. Food is used to control for potential nonspecific, ataxis effects of the drugs and combinations. The ideal drug or drug combination is one that reduces cocaine self-administration without affecting food-maintained responding. The other preclinical model we use is the cue-induced reinstatement of extinguished cocaine seeking model of relapse. In this model, rats are trained to self-administer cocaine, and the ability of conditioned cues in the environment to reinstate extinguished responding is assessed and taken as a measure of relapse.

More specifically, adult male Wistar rats are implanted with chronic jugular catheters. Following recovery from surgery, the rats are trained to respond under a multiple, alternating schedule of food reinforcement and cocaine self-administration. Food-maintained responding is used to control for the non-specific motor effects of the various treatments. During the food component of the schedule, the stimulus light located above the food response lever is illuminated to indicate the availability of food reinforcement. Initially, each depression of the food response lever results in a brief darkening of the food stimulus light (0.6 seconds) and the delivery of a food pellet (45 mg). A 25-second timeout follows the delivery of each food pellet. During this timeout, the stimulus light is darkened and responses on the food lever are counted but have no scheduled consequences. Responding on the other (cocaine) lever during the food component also has no scheduled consequences. The response requirement for the food lever is gradually increased over several sessions from continuous reinforcement to a fixed-ratio four schedule whereby four responses were required for food presentation. Following 15 minutes of access to food, all stimulus lights in the chamber are darkened for a 1-minute timeout. Following the timeout, the stimulus light above the cocaine response lever is illuminated to indicate the availability of cocaine (0.125, 0.25, or 0.5 mg/kg/infusion). Initially, each depression of the cocaine response lever results in a brief darkening of the stimulus light and an infusion of cocaine (200 μl delivered over 5.6 seconds). A 20-second timeout period follows each infusion. The response requirement for cocaine is gradually increased to a fixed-ratio four schedule of reinforcement. After 15 minutes of access to cocaine and a 1-minute timeout, the rats are again allowed 15 minutes access to the food component of the schedule. Access to food and cocaine alternates in this manner every 15 minutes during the two hour behavioral sessions so that each rat is exposed to food and cocaine for four 15-minute periods each. Each behavioral session begins with 15 minutes access to either food or cocaine, and this alternates daily. Stable baselines of response are established when the total number of cocaine and food presentations, as well as the number of presentations during each of the four exposures each session, varies less than 10% for three consecutive sessions. At least three different doses of cocaine (e.g., 0.125, 0.25, and 0.5 mg/kg/infusion) are tested. Rats are first trained to self-administer 0.25 mg/kg/infusion, our standard dose of cocaine. When responding stabilizes, the dose is changed to 0.125 or 0.5 mg/kg/infusion as appropriate. We have found that initially training rats with this moderated dose of cocaine (i.e., 0.25 mg/kg/infusion) hastens stability with the lower dose (i.e., 0.125 mg/kg/infusion).

Once stable baselines of responding are obtained, dose-response curves for the various compounds are individually generated for each rat. Rats are treated with each dose at least twice with a minimum of two days of baseline cocaine self-administration interspersed between each test. Each group of rats is tested with only two of the test combinations to minimize potential carryover effects. The minimally effective dose that reduces cocaine self-administration by at least 50% without affecting food-maintained responding (i.e., the high dose) is determined for each of the test combinations. In some experiments, the dose selected for the test combination experiments is one-half of the minimally effective dose, and this dose has to also produce less than a 10% decrease in cocaine self-administration (i.e., an ineffective dose). If one-half of the minimally effective dose reduces cocaine self-administration by more than 10%, then the dose is once again reduced by one-half. For example, 12.5 mg/kg metyrapone has previously been successfully used in studies with alprazolam and oxazepam. This dose (12.5 mg/kg) has no effect on cocaine- or food-maintained responding when tested alone, but significantly reduces cocaine self-administration when combined with a similarly ineffective dose of alprazolam (i.e., 1.0 mg/kg, ip) or oxazepam (10 mg/kg, ip). This rationale guides the selection of the doses of each of the compounds in the combination studies. Each experimental group consists of between 8 and 10 rats.

### Example 4

Training to self-administer nicotine or methamphetamine: Training to self-administer nicotine or methamphetamine is performed essentially using the same protocols as described above in “Example 3.” The doses of nicotine or methamphetamine used are determined on the basis of literature and experiments that provide doses that are within 10 times the minimal doses that can induce successful self-administration behavior.

### Example 5

Cue-induced Reinstatement of Extinguished Addictive Substance Seeking: The experiments described herein are designed to investigate whether or not the test combinations identified as effective in reducing addictive substance self-administration would also block the ability of conditioned cues to reinstate extinguished addictive substance-seeking behavior. Adult male Wistar rats are implanted with chronic jugular catheters and trained to self-administer the addictive substance by pressing one of the response levers in the experimental chamber (i.e., the “active” or “addictive substance” lever) under a fixed-ratio four (FR4) schedule of reinforcement during daily 2-hour sessions conducted 5 days per week. At the start of each session, both levers are extended into the chamber and the stimulus light above the active lever is illuminated to indicate the availability of the addictive substance. Initially, each depression of the active lever results in an intravenous infusion of the addictive substance and the concurrent presentation of a house light and tone compound stimulus (i.e., the conditioned cue or secondary reinforcer). A 20-second timeout period follows each infusion. The stimulus light above the active lever and the house light and tone compound stimulus are extinguished during the timeout period, and the light above the active lever is illuminated once the timeout ended. When responding on the active lever varies less than 20% for two consecutive days, the response requirement is increased to FR2. When similar stability is observed under the FR2 schedule or reinforcement, the response requirement is increased to the final ratio of four. The criteria for stable responding under the FR4 schedule of reinforcement is a minimum of 10 days of exposure to this schedule that concludes with at least three consecutive days when responding varies by less than 10%. Responses on the inactive lever are counted, but results in no programmed consequences at any time. Once stable addictive substance self-administration is observed, rats are exposed to extinction; the rats are placed into the behavioral chambers, but responding on the “addictive substance” (active) lever produces no programmed consequences. Extinction training continues until responding decreases to less than 20% of baseline self-administration. Then reinstatement testing is commended. The rats are placed into the experimental chambers, both response levers are extended into the chamber, and the stimulus light above the “active” lever is illuminated as during self-administration training. During reinstatement, responding on the “active” lever results in a 5.6-second presentation of the conditioned reinforcer (i.e., the house light and tone compound stimulus that has been paired with addictive substance during self-administration). Responses on the “inactive” lever are counted but results in no scheduled consequences. Responding on the “active” lever during reinstatement testing is taken as an index of addictive substance-seeking behavior. Each experimental group consists of 8 to 10 rats.

### Example 6

Pharmacokinetic interaction between addictive substances and the test combinations: Adult male Wistar rats (90 to 120 days old) are implanted with chronic, indwelling jugular catheters and are allowed to recover from surgery. On the test day, the rats are pretreated with intra-peritoneal injections of the test combinations or vehicle 30 minutes before the addictive substance injections are administered. The test combinations are selected from our behavioral studies that demonstrated that these combinations reduced addictive substance self-administration or the cue-induced reinstatement of extinguished addictive substance seeking without affecting food-maintained responding. Thirty minutes following the test combination or vehicle injection, the rats receive intravenous injections of addictive substance every 2 minutes for 1 hour. After the final injection of the addictive substance, blood is collected from the catheter for the analysis of the addictive substance and its metabolites. Concentrations of the first agent and/or the second agent are also determined. All the drug, addictive substance and metabolite concentrations are determined using GCMS procedures.

### Example 7

The forced swim test, an animal model of depression: The Forced Swim Test (FST) is an animal-model that possesses predictive validity for assessing a drug's anti-depressive efficacy. The subject is exposed to an inescapable, life-threatening situation to elicit learned helplessness. To achieve this, rats are placed in a cylinder filled with water from which they cannot escape and in which they must swim to stay afloat. At a point in time when the rat ‘realizes’ its situation is hopeless, despair-like behavior appears and rather than attempting to escape or swim, the rat becomes immobile. The time in this immobility posture is the behavior that is measured as despair. The potential antidepressant properties of the test combinations are evaluated in male Wistar rats using the FST. Rats are injected with one of the test combinations both on day one after testing and again on day two before testing (acute) or for fourteen days before initiating testing on day one (chronic). The acute and chronic administrations of the drugs, alone and in combination, are effective in reducing immobility in the FST, indicating that this pharmacotherapy has antidepressant activity.

Learned helplessness is the construct on which the validity of using the FST as a model of depression is based. In humans, learned helplessness is often manifested as a symptom of depression, which appears as a loss of coping ability. For that reason we believe that drugs that have the effect of decreasing the time of immobility in the FST have potential as candidates for lessening the loss of coping ability seen in the human model of depression. In the current studies, test combinations are checked alone and in combination in the FST to determine whether these agents might show antidepressant activity.

The parameters of the study are outlined above. More specifically, male Wistar 20 rats from Harlan weighing 275-400 grams are used. The rats are allowed to acclimate at least one day in the Animal Resources Facility after arrival before being tested. To perform the FST, a Plexiglas cylinder (40 cm tall x 18 cm diameter) is filled with fresh, 25° C. water to a depth of 20 cm, which is deep enough so the rat cannot touch bottom, yet far enough from the rim to prevent the rat from escaping. Rats are injected intra-peritoneally with either vehicle, drugs, or the test combinations on day one after testing and again on day two before testing (acute) or for fourteen days before initiating testing on day one (chronic). On day one, the rat is removed from his cage, placed in the water, and observed for fifteen minutes. Generally, for the first few minutes, the rat would swim around with his paws thrashing above the water line, sniff, dive, and attempt to jump out of the cylinder. Such actions are deemed escape-oriented behavior. Following the escape-oriented behavior is a time characterized by the rat discontinuing its attempts to escape. Generally, the rat would either tread water, exerting only enough energy to keep its head above water, or would float with only its nose above the water line. This second phase of behavior is deemed the immobility posture. Length of time spent in escape-oriented behavior and immobility posture is recorded. Then the rat is removed from the water, dried with a towel, and returned to his home cage. On day two, the procedure is repeated for five minutes and the time spent engaging in escape-oriented behavior and immobility posture are recorded. The second day's duration of immobility is compared among the different groups. Dosage groups are compared to the vehicle-injected controls using a one way ANOVA with p<0.05. If the Immobility Time for a test combination group is statistically significant compared to that of the vehicle group, the drug combination is considered to exhibit an antidepressant-like effect.

A number of embodiments of the invention have been described. Nevertheless, it will be understood that various modifications may be made without departing from the spirit and scope of the invention. Accordingly, other embodiments are within the scope of the following claims.

1. A pharmaceutical composition comprising a first agent and a second agent; wherein the first agent is selected from a CRF-1 antagonist and a cortisol inhibitor; and
wherein the second agent is selected from the group consisting of: a selective serotonin reuptake inhibitor (SSRI), a beta blocker, an antipsychotic, an azapirone, and an alpha-adrenergic agonist.

2. The pharmaceutical composition of claim 1, wherein the CRF-1 antagonist is pexacerfont or verucerfont.

3. The pharmaceutical composition of claim 1, wherein the cortisol inhibitor is fluconazole.

4. The pharmaceutical composition of claim 1, wherein the first agent is

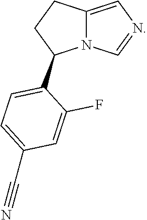 [SMILES: N#Cc1ccc([C@H]2CCc3cncn32)c(F)c1]

5. The pharmaceutical composition of claim 1, wherein the SSRI is escitalopram.

6. The pharmaceutical composition of claim 1, wherein the beta blocker is metoprolol.

7. The pharmaceutical composition of claim 1, wherein the antipsychotic is ariprazole.

8. The pharmaceutical composition of claim 1, wherein the azapirone is buspirone.

9. The pharmaceutical composition of claim 1, wherein the alpha-adrenergic agonist is clonidine.

10. The pharmaceutical composition of claim 1, wherein the first agent is pexacerfont; and the second agent is selected from the group consisting of:
escitalopram, metoprolol, ariprazole, buspirone, and clonidine.

11. The pharmaceutical composition of claim 1, wherein the first agent is verucerfont; and the second agent is selected from the group consisting of:
escitalopram, metoprolol, ariprazole, buspirone, and clonidine.

12. The pharmaceutical composition of claim 1, wherein the first agent is fluconazole; and the second agent is selected from the group consisting of:
escitalopram, metoprolol, ariprazole, buspirone, and clonidine.

13. The pharmaceutical composition of claim 1, wherein the composition is selected from one of the following combinations:

| Combination | | | Combination | | |
|---|---|---|---|---|---|
| No. | Compound 1 | Compound 2 | No. | Compound 1 | Compound 2 |
| 29 | verucerfont | escitalopram | 652 | verucerfont | metoprolol |
| 53 | verucerfont | ariprazole | 74 | verucerfont | buspirone |
| 72 | verucerfont | clonidine | 605 | pexacerfont | escitalopram |
| 664 | pexacerfont | metoprolol | 629 | pexacerfont | ariprazole |
| 650 | pexacerfont | buspirone | 648 | pexacerfont | clonidine |
| 557 | fluconazole | escitalopram | 663 | fluconazole | metoprolol |
| 581 | fluconazole | ariprazole | 602 | fluconazole | buspirone |
| 600 | fluconazole | clonidine | | | |

14. The pharmaceutical composition of claim 1, wherein the composition is formulated for administration by one or more of the group consisting of: oral, rectal, parenteral, topical, intradermal, subcutaneous, intramuscular, intravenous, intraosseous, intraperitoneal, intrathecal, epidural, intracardiac, intraarticular, intracavernous, intravitreal, intravaginal, intracervical and inhalation routes.

15. The pharmaceutical composition of claim 14, wherein the composition is formulated one or more of the following dosage forms: a liquid, solution, suspension, emulsion, elixir, syrup, drop, powders electuary, granule, capsule, tablet, lozenge, pastille, gel, paste, ointment, cream, lotion, oil, foam, spray, mist, or aerosols.

16. (canceled)

17. A method of treating a patient who is suffering from a disorder associated with aberrant activity in the HPA axis, the method comprising administering to the patient a therapeutically effective amount of a composition of claim 1.

18. The method of claim 17, wherein the disorder is an addiction to a substance, addiction to an activity, substance use disorders, mood disorders, anxiety disorders, bipolar disorder, sleep disorders, insomnia, posttraumatic stress syndrome, borderline personality disorder, disruptive behavior disorders, ADHD, major depressive disorder, burnout, chronic fatigue syndrome, fibromyalgia, irritable bowel syndrome, eating disorders, obesity, depression, menopause, premenstrual syndrome (PMS), obsessive compulsive disorder (OCD),social anxiety, generalized anxiety disorder, dysthymia, or schizophrenia.

19. (canceled)

20. (canceled)

21. The method of claim 18, wherein the substance is cocaine, amphetamines, methamphetamine, methylphenidate, heroin, codeine, hydrocodone, nicotine, alcohol, prescription medication, marijuana, tobacco, methadone, or food.

22. The method of claim 18, wherein the activity is gambling, sex, or eating.

23. The method of claim 18, wherein the eating disorder is Prader Willi Syndrome.

24. (canceled)

25. (canceled)

26. (canceled)
